(19) United States
(12) Patent Application Publication
(10) Pub. No.: US 2017/0260185 A1
(43) Pub. Date: Sep. 14, 2017

(54) 2-[BIS(4-FLUOROPHENYL)METHYL]-2,7-DIAZASPIRO[4.5]DECAN-10-ONE DERIVATIVES AND RELATED COMPOUNDS AS INHIBITORS OF THE HUMAN DOPAMINE-ACTIVE-TRANSPORTER (DAT) PROTEIN FOR THE TREATMENT OF E.G. ATTENTION DEFICIT DISORDER (ADD)

(71) Applicant: SHIRE INTERNATIONAL GMBH, Zug (CH)

(72) Inventors: Teresa SEMERARO, Verona (IT); Luca TARSI, Verona (IT); Fabrizio MICHELI, Verona (IT); Tim LUKER, Nottingham (GB); Susanna CREMONESI, Verona (IT)

(21) Appl. No.: 15/511,420

(22) PCT Filed: Sep. 14, 2015
(86) PCT No.: PCT/IB2015/057029

§ 371 Date: Mar. 15, 2017

(30) Foreign Application Priority Data
Sep. 16, 2014 (GB) 1416346.3

(51) Int. Cl. C07D 471/10; C07D 498/10; C07D 513/10; C07D 491/20

(52) U.S. Cl. CPC C07D 471/10; C07D 491/20; C07D 498/10; C07D 513/10

(57) ABSTRACT

The present invention provides compounds of formula (I) and in particular 2-[bis(4-fluorophenyl)methyl]-2,7-diazaspiro[4.5]decan-10-one derivatives and related compounds as inhibitors of human dopamine-active-transporter (DAT) protein for the treatment of sexual dysfunction, affective disorders, anxiety, depression, chronic fatigue, Tourette syndrome, Angelman syndrome, attention deficit disorder (ADD), attention deficit hyperactivity disorder (ADHD), obesity, pain, obsessive-compulsive disorder, movement disorders, CNS disorders, sleep disorders, narcolepsy, conduct disorder, substance abuse (including smoking cessation), eating disorders, and impulse control disorders.

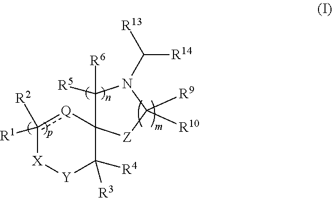

This invention relates to spirocyclic derivatives that are inhibitors of dopamine active transporter protein (DAT) and to pharmaceutical compositions containing, and the uses of, such derivatives.

### BACKGROUND TO THE INVENTION

The spirocyclic derivatives of the present invention are inhibitors of human dopamine active transporter protein (DAT) and have a number of therapeutic applications, particularly in the treatment of sexual dysfunction, affective disorders, anxiety, depression, chronic fatigue, Tourette syndrome, Angelman syndrome, attention deficit disorder (ADD), attention deficit hyperactivity disorder (ADHD), obesity, pain, obsessive-compulsive disorder, movement disorders, CNS disorders, sleep disorders, narcolepsy, conduct disorder, substance abuse (including smoking cessation), eating disorders, and impulse control disorders.

Dopamine (DA) is a neurotransmitter which has a fundamental role in cognitive, affective, motor, motivational and reward-related functions. Following evoked action potentials DA is released into the synaptic cleft and this DA signal is extinguished by reuptake of DA into pre-synaptic neurons by DAT and by amine diffusion and local metabolism via enzymatic degradation. Dysfunction of the dopaminergic system is implicated in numerous CNS disorders and consequently DAT has been the focus of research into a number of these conditions and strong associations exist between abnormal DAT expression and/or function and disease.

Several marketed drugs have pharmacological activity at DAT, but none are selective and potent DAT inhibitors. Stimulants such as amphetamine and methylphenidate have multiple pharmacological activities including effects on synaptic levels of DA, noradrenaline (NE) and serotonin (5-HT). Despite their therapeutic potential in conditions such as ADHD, they also carry unwanted side effects such as abuse potential (1), cardiovascular effects (2), appetite suppression (3) and sleep disturbance (4).

Other non-selective DAT inhibitors are also used to treat CNS disorders. Bupropion which is prescribed as an antidepressant and a smoking cessation aid has a significant DAT component to its pharmacological activity, although it carries an increased seizure risk. Similarly Modafinil which is prescribed as a treatment for narcolepsy, excessive daytime sleepiness and shift work sleep disorder has been shown to inhibit DAT as part of its pharmacological mechanism of action. Multiple compounds have been developed that target the other monoamine transporters either selectively as inhibitors of the serotonin transporter (SERT) (Citalopram, Fluoxetine) or noradrenaline transporter (NET) inhibitors (Atomoxetine, Reboxetine) as well as dual serotonin/noradrenaline reuptake inhibitors (Venlafaxine). Drugs that inhibit SERT and NET have been burdened with multiple adverse side effects such as nausea (5), sexual dysfunction (6), increased suicide risk (7) for drugs that elevate 5-HT levels and elevated heart rate and blood pressure (8, 9) for drugs that increase noradrenaline levels. This makes a selective and potent DAT inhibitor, with a neurochemical profile distinct from that of stimulants, a highly desirable compound for the treatment of CNS disorders.

ADD and ADHD are neurodevelopmental psychiatric, behavioural and cognitive disorders characterised by concentration deficits, inner restlessness/hyperactivity, and impulsivity. These are the most common behavioural disorders amongst children, with a prevalence of 5-10% of the general population. It is widely believed that the symptoms of these disorders result from a dopaminergic and/or noradrenergic hypofunction. There is a wealth of information showing that the core symptoms of ADHD are influenced by changes in dopaminergic function (10) and hence a DAT inhibitor which would raise synaptic DA levels, should be efficacious. Current treatments for ADD/ADHD include the stimulants amphetamine and methylphenidate. These compounds have pharmacological activity for DAT, amongst other activities, and it is believed that their efficacy is derived from the elevation of corticostriatal DA and NE. These drugs are not selective DAT inhibitors however, and as such cause rapid, transient and marked release of DA from synaptic terminals which has been associated with their unwanted side effects, such as abuse potential. This neurochemical profile is distinct from that of a selective and potent DAT inhibitor which causes a slower increase in dopamine which is sustained for a much longer duration. This different neurochemical profile has been associated with less reinforcing effects and subsequently lower abuse potential (11). In addition to the neurochemical evidence for a likely therapeutic benefit of DAT inhibitors in ADHD, several studies have shown associations between DAT polymorphisms and overexpression of DAT in ADHD (12). Preclinical models of ADHD symptoms have shown that like amphetamine and methylphenidate a selective DAT inhibitor will decrease impulsive behaviour in rodents (13) further supporting the potential for efficacy of DAT inhibitors. Collectively this evidence provides compelling data to believe that selective DAT inhibitors will be efficacious in ADD/ADHD and other disorders characterised by poor impulse control (such as Trichotillomania, pathological gambling, Kleptomania and disorders with comorbid impulse control such as Parkinson's disease) or inattention.

Tourette's syndrome is a neuropsychiatric disorder characterised by motor and/or phonic tics. It normally presents during childhood and is poorly treated with drugs. Studies have postulated that one aspect underlying Tourette's is dopaminergic dysfunction whereby tonic/phasic dysfunction results in reduced synaptic DA levels and consequently higher levels in axon terminals leading to increased stimulus dependent release. Further studies have shown that post-mortem tissue from Tourette's patients showed elevated levels of DAT in the frontal lobe (14) and that polymorphisms in DAT are associated with the occurrence of Tourette's. This was further supported in a clinical study of drug naïve children which showed and increased specific/non-specific DAT binding ratio in those with Tourette's (15). These findings suggest that a selective DAT inhibitor may provide symptomatic relief for Tourette's patients.

Other neuropsychiatric disorders such as obsessive compulsive disorder (OCD), oppositional defiant disorder (ODD) and conduct disorder have also been associated with DAT. OCD patients have been shown to have an increased specific/non-specific DAT binding ratio (16) and this ratio was altered following treatment with SSRIs which are commonly used to treat OCD. Similarly abnormal dopamine function and/or dopamine turnover have been implicated in ODD, conduct disorder and other related behavioural disorders (17) and polymorphisms in DAT have been implicated as a risk factor for externalising behaviour in children. Studies showing that children with conduct disorder display disrupted reinforcement signalling and a response to reward have also suggested that modulation of synaptic dopamine levels could be a therapeutic option for these disorders presenting the opportunity to use a selective DAT inhibitor to treat these behavioural disorders.

Sleep disorders such as narcolepsy, cataplexy, excessive daytime sleepiness and shift work sleep disorder can interfere with an individual's normal mental and physical wellbeing. Several of these disorders are treated with drugs that have pharmacological activity at DAT. Modafinil is widely used to treat narcolepsy and its therapeutic potential has been related to occupancy of DAT). Other treatments for sleep disorders include amphetamine, methamphetamine and methylphenidate, all of which have pharmacological actions at DAT. Preclinical studies have shown that the wake promoting effects of several of these compounds and a selective DAT inhibitor are abolished in DAT knockout mice. Together these data support the use of a selective DAT inhibitor in the treatment of sleep disorders.

Mood disorders such as major depressive disorder, bipolar depression, seasonal affective disorder, melancholic depression, catatonic depression, postpartum depression and dysthymia represent a major medical and social burden on society and are amongst the most common of all CNS disorders. Treatment for these disorders is currently inadequate with low levels of efficacy and poor responder rates to currently available therapies. In addition many of the drugs that are the current standard of care carry unwanted side effects. SPECT studies in patients suffering from major depressive disorder have shown that there is an increased binding of DAT in depressed patients and that this was reversed following successful antidepressant treatment (18,19). In addition to this marketed antidepressants such as Nomifensine have a significant DAT inhibitory component to their mechanism of action. Preclinical studies investigating the behavioural phenotype of DAT knockout mice in tests for antidepressant activity have shown that genetic removal of DAT function results in antidepressant-like behaviour. This evidence is supportive for a therapeutic benefit for DAT inhibitors in mood disorders.

A comorbid symptom of depression and an unwanted side effect of many commonly used antidepressants is sexual dysfunction (20). Bupropion a commonly prescribed antidepressant with a significant DAT inhibitory component to its mechanism of action has been shown to result in fewer sexual dysfunction related side effects than other antidepressants (21). Furthermore Bupropion has been shown to reverse the sexual dysfunction caused by SSRIs. Preclinical studies have shown an effect of Bupropion on sexual behaviour in rats which is supported by clinical evidence that the drug is effective in treating women suffering from hypoactive sexual desire disorder. Amphetamine has also been shown to increase sexual behaviour in male and female rats and has also been shown to reverse sexual impairment in female rats. This evidence for drugs that have pharmacological activity at DAT is an indicator that a selective and potent DAT inhibitor would be a suitable therapy for antidepressant induced sexual dysfunction as well as for treating sexual dysfunction in non-depressed patients.

DAT polymorphisms have been implicated in anxiety disorders such as post traumatic stress disorder (PTSD) (22). The non-selective monoamine oxidase inhibitor Phenelzine which elevates dopamine levels in the brain amongst its actions has been shown to reduce the symptoms of PTSD. Bupropion which has a significant DAT inhibitory component to its mechanism of action is also prescribed for patients with anxiety disorders and has been shown to be efficacious in patients with panic disorder, further supporting the potential of DAT inhibitors in these conditions.

Movement disorders such as Parkinson's disease (PD) and Restless Leg Syndrome (RLS) are common neurological disorders which have been treated with therapies that result in elevated brain dopamine. PD is characterised by a loss of dopaminergic neurones in the nigrostriatal pathway and a subsequent loss of dopamine. Drugs such as L-DOPA which is converted to dopamine in the brain have been shown to alleviate the motor symptoms of both PD and RLS. Given that DAT inhibitors also increase dopamine levels it is reasonable to assume that they would also provide therapeutic benefit in movement disorders which have been shown to have a dopaminergic component. Further support for this hypothesis is given by the fact that methylphenidate, a stimulant which has DAT inhibition amongst its pharmacological activities has shown to be clinically efficacious in PD patients, both in motor (23) and non-motor symptoms (24,25).

Addiction and substance abuse are closely linked to dopamine and reward circuits in the brain. These substance dependencies include alcohol dependence, opioid dependence, cocaine dependence, cannabis dependence, amphetamine dependence (or amphetamine-like), hallucinogen dependence, inhalant dependence, polysubstance dependence, phencyclidine (or phencyclidine-like) dependence, and nicotine dependence. Preclinical studies using the selective DAT inhibitor GBR12909 and other benztropines have shown that these compounds can block the rewarding effects of drugs of abuse, such as cocaine. GBR12909 has been shown to block the neurochemical effects of cocaine (26, 27) as well as that of amphetamine. Furthermore compounds which have been demonstrated to be DAT inhibitors are effective in smoking cessation. This provides evidence that a high affinity, selective DAT inhibitor could block the rewarding effects of drugs of abuse and be an effective medication to treat addiction.

Dopamine is also known to have a role in eating disorders such as Binge Eating Disorder (BED). Eating disorders such as BED are known to have multiple components including impulse control, reward circuits and cognition, all of which are under the influence of dopaminergic signalling. It has been shown that BED sufferers have abnormal brain dopamine responses, which regulates motivation for food intake (28). In addition BED and obese patients show an abnormal frontostriatal dopamine signalling as compared to healthy controls (29). Preclinical models have shown that stimulation of the nucleus accumbens, which receives major dopaminergic input, attenuates binge eating behaviour in rats and that this effect is blocked by dopaminergic antagonists. This indicates that increased synaptic dopamine is a potential therapeutic opportunity for eating disorders such as binge eating disorder. Preclinical data has shown that food intake is modulated by drugs which modulate synaptic dopamine levels and specifically by compounds with affinity at DAT (30). DAT has been specifically implicated in BED and other eating disorders due to polymorphisms in DAT being associated with eating disorders (31). This hypothesis is further supported by the efficacy of drugs with DAT inhibition as part of their mechanism of action in clinical trials of BED and other eating disorders (32). Together this is supportive for the therapeutic potential of a selective DAT inhibitor in eating disorders such as BED.

Dopamine has a well-documented role in cognition and particularly in cognitive deficits seen in patients suffering from diseases characterised by abnormal dopaminergic signalling such as Parkinson's disease and schizophrenia (33). This coupled with the fact that cortical dopamine D1 receptor function is linked to NMDA mediated glutamate signalling implies that cognitive processes would be expected to be enhanced by DAT inhibitors.

Chronic or persistent fatigue is a symptom which is common to several diseases and can be persisting or relapsing (34). Disease states that are associated with fatigue include chronic fatigue syndrome, post-viral fatigue syndrome, HIV, multiple sclerosis, amyotrophic lateral sclerosis (ALS), myasthenia gravis, sarcoidosis, cancer, chemotherapy treatment, celiac disease, irritable bowel syndrome, spondyloarthropathy, fibromyalgia, arthritis, infectious diseases, diabetes, eating disorders, Parkinson's disease, sleep disorders, stroke, mood disorders, drug and alcohol abuse. Clinical studies have shown that multiple drugs with DAT inhibition as part of their mechanism of action are effective in combating fatigue in chronically ill patients (35). Drugs such as modafinil, methylphenidate and bupropion which share DAT inhibition as a common pharmacological mechanism of action have been shown to be efficacious in fatigue associated with cancer, chemotherapy, sarcoidosis, ALS, depression, bipolar disorder, multiple sclerosis, Parkinson's disease, HIV and chronic fatigue syndrome. This evidence is supportive of likely efficacy for a selective and potent DAT inhibitor in fatigue associated with the diseases mentioned above.

The multiple potential applications for a selective and potent DAT inhibitor have resulted in numerous chemical series being described in the literature. A particular issue has been pharmacological selectivity, with many previously described structural classes of DAT inhibitors suffering from significant off target pharmacology, which has limited their development. A particular issue is the affinity of DAT inhibitors described in the literature for ion channels. Vanoxerine has been shown to have significant activity at multiple ion channels resulting in a cardiovascular safety risk that has hampered its development (36). The compound showed potent functional activity at multiple sodium, calcium and potassium channels which would be an undesirable profile for a drug to treat CNS disorders. In addition to off target ion channel pharmacology DAT inhibitors (particularly those of the benztropine class) have been shown to have pharmacological activity at multiple other receptors such as the serotonin receptor 5-HT2, the muscarinic receptor M1 and the histamine receptor H1 (37,38,39). These significant secondary pharmacological activities may introduce unwanted side effects to potentially therapeutically beneficial DAT inhibitors. This makes the selectivity profile of DAT inhibitors of particular importance.

Therefore there remains a need to develop new DAT inhibitors, especially inhibitors that are selective over noradrenaline and serotonin, that will have utility to treat a wide range of disorders, in particular to treat depression, ADHD and eating disorders. Preferred compounds will possess a good pharmacokinetic profile and in particular will be suitable as drugs for oral delivery. Particularly preferred compounds will additionally display selectivity over noradrenaline and serotonin.

### SUMMARY OF THE INVENTION

The present invention relates to a series of spirocyclic derivatives that are inhibitors of DAT. Many of these compounds demonstrate good selectivity for DAT and are potentially useful in the treatment of sexual dysfunction, affective disorders, anxiety, depression, Tourette syndrome, Angelman syndrome, attention deficit disorder (ADD), attention deficit hyperactivity disorder (ADHD), obesity, pain, obsessive-compulsive disorder, movement disorders, CNS disorders, sleep disorders, narcolepsy, conduct disorder, substance abuse (including cocaine abuse and smoking cessation), eating disorders, chronic fatigue and impulse control disorders. The invention further relates to pharmaceutical compositions of the inhibitors, to the use of the compositions as therapeutic agents, and to methods of treatment using these compositions.

In an aspect, the invention provides a compound according to formula I

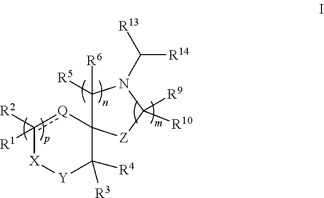

wherein:
Q is selected from CR⁷R⁸, C═O, C═N—OH, C═N—O-alkyl, NH, N-cycloalkyl, N-alkyl, S(O)_{q} and O;
X is selected from C═O, CR¹¹R¹², NH, N-cycloalkyl and N-alkyl;
Y is selected from CR¹¹R¹², NH, N-alkyl, N-cycloalkyl, S(O)_{q} and O;
   wherein:
      X is C═O or CR¹¹R¹² when Y is O, S(O)_{q}, NH, N-alkyl or N-cycloalkyl;
      X is C═O or CR¹¹R¹² when p is 0 and Q is S(O)_{q}, O, NH, N-cycloalkyl or N-alkyl;
      Y is CR¹¹R¹² when X is NH, N-cycloalkyl or N-alkyl;
      Q is selected from CR⁷R⁸, C═O, C═N—OH and C═N—O-alkyl when n is 0;
      Q is CR⁷R⁸ when p is 0 and X is NH, N-cycloalkyl or N-alkyl;
      Q is CR⁷R⁸, O, NH, N-cycloalkyl or N-alkyl when p is 0 and X is C═O;
      Q is NH, N-cycloalkyl or N-alkyl and X is CR¹¹R¹² when Y is O or S(O)_{q}, and
      at least one of O, X and Y is NH, N-cycloalkyl or N-alkyl;
Z is selected from CR¹¹R¹², O and S; wherein Z is CR¹¹R¹² when Q is O, S(O)_{q}, NH, N-cycloalkyl or N-alkyl, or when m is 0, or when n is 0;
R¹ is selected from H, OH, alkyl, F, Cl, and alkoxy;
R² is selected from H, OH, alkyl, F, Cl, and alkoxy;
   or R¹ and R² may together form ═O;
R³ and R⁴ are independently selected from H, OH, alkoxy and alkyl;
   or R³ and R⁴ may both be O, wherein said O atoms are linked by an alkylene group to form a straight chain or branched alkylenedioxy group;
   or R³ and R⁴ may together form ═O;
R⁵ and R⁶ are independently selected from H and alkyl;
   or R⁵ and R⁶ may together form ═O;
R⁷ is selected from H, F, Cl, OH and alkoxy;
R⁸ is absent or is selected from H, F, Cl, OH and alkoxy;
   or R⁷ and R⁸ may both be O, wherein said O atoms are linked by an alkylene group to form an alkylenedioxy group;
R¹³ is substituted phenyl;
   R¹⁴ is substituted phenyl or unsubstituted phenyl;
R⁹, R¹, R¹¹, R¹², R¹⁵ and R¹⁶ are independently selected from H and alkyl;
q is 0, 1 or 2;
n is 0, 1 or 2, wherein n is 0 or 1 when m is 2, and n is 1 or 2 when m is 0
m is 0, 1 or 2, wherein m is 0 or 1 when n is 2, and m is 1 or 2 when n is 0;
p is 0, 1 or 2; wherein p is 1 or 2 when n is 2;
- - - - is absent or represents a bond; wherein when - - - - is a bond R² is absent, Q is CR⁷R⁸, R⁸ is absent, and p is 1 or 2;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1, 2, 3, 4 or 5 substituents independently selected from cycloalkyl, S-alkyl, S(O)alkyl, S(O)₂alkyl, cycloalkyl, heterocyclyl, alkoxy, OH, —CN, CF₃, COOR¹⁵, CONR¹⁵R¹⁶, F, Cl, NR¹⁵COR¹⁶ and NR¹⁵R¹⁶;
alkylene is a bivalent C₁₋₃ straight-chained alkyl radical or a bivalent C₃₋₄ branched alkyl radical, wherein alkylene may optionally be substituted with 1 or 2 substituents selected from S-alkyl, S(O)alkyl, S(O)₂alkyl, heterocyclyl, alkoxy, OH, —CN, CF₃, COOR¹⁵, CONR¹⁵R¹⁶, F, Cl, NR⁵COR¹⁶ and NR¹⁵R¹⁶;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1, 2, 3, 4 or 5 substituents independently selected from S-alkyl, S(O)alkyl, S(O)₂alkyl, alkyl, OH, —CN, CF₃, COOR¹⁵, CONR¹⁵R¹⁶, F, Cl, NR¹⁵COR¹⁶ and NR¹⁵R¹⁶;
cycloalkyl is a monocyclic saturated hydrocarbon of between 3 and 7 carbon atoms; cycloalkyl may optionally be substituted with 1, 2, 3, 4 or 5 substituents independently selected from S-alkyl, S(O)alkyl, S(O)₂alkyl, alkyl, alkoxy, OH, —CN, CF₃, COOR¹⁵, CONR¹⁵R¹⁶, F, Cl, NR¹⁵COR¹⁶ and NR¹⁵R¹⁶;
substituted phenyl is a phenyl group substituted with 1, 2 or 3 substituents independently selected from alkyl, cycloalkyl, heterocyclyl, alkoxy, S-alkyl, S(O)alkyl, S(O)₂alkyl, OH, F, Cl, —CN, OCF₃, CF₃, NR¹³COR¹⁴ and NR¹⁵R¹⁶;
heterocyclyl is a monocyclic ring which is saturated or partially unsaturated, containing, where possible, 1 or 2 ring members independently selected from N, S, O and NR¹⁵ and 2 to 5 carbon atoms; heterocyclyl may optionally be substituted with 1, 2 or 3 substituents independently selected from alkyl, cycloalkyl, alkoxy, S-alkyl, S(O)alkyl, S(O)₂alkyl, oxo, OH, F, Cl, —CN, OCF₃, CF₃, NR¹⁵COR¹⁶ and NR¹⁵R¹⁶;
and tautomers, stereoisomers (including enantiomers, diastereoisomers and racemic and scalemic mixtures thereof), pharmaceutically acceptable salts and solvates thereof;
wherein:
R¹ is not OH or alkoxy when Q is NH, N-alkyl, N-cycloalkyl or when X is NH, N-alkyl or N-cycloalkyl; and
R² is not OH or alkoxy when Q is NH, N-alkyl, N-cycloalkyl or when X is NH, N-alkyl or N-cycloalkyl; and
R³ is not OH or alkoxy when Y is O, NH, N-alkyl or N-cycloalkyl; and
R⁴ is not OH or alkoxy when Y is O, NH, N-alkyl or N-cycloalkyl.

In an aspect, the invention provides a compound of formula I wherein R¹³ and R¹⁴ are para-fluoro-phenyl.

In an aspect, the invention provides a compound of formula I wherein p is 1.

In an aspect, the invention provides a compound of formula I wherein m is 1 or 2 and n is 1 or 2, wherein n is 1 when m is 2; and m is 1 when n is 2.

In an aspect, the invention provides a compound of formula I wherein n is 1 and m is 1.

In an aspect, the invention provides a compound of formula I wherein Z is CH₂.

In an aspect, the invention provides a compound of formula I wherein X is CH₂ and Y is NH.

In an aspect, the invention provides a compound of formula I wherein Q is selected from CR⁷R⁸, S and O.

In an aspect, the invention provides a compound of formula I wherein Q is CR⁷R⁸.

In an aspect, the invention comprises a compound selected from Examples 1 to 32.

In yet another aspect the present invention provides an N-oxide of a compound of formula I as herein defined, or a prodrug or pharmaceutically acceptable salt thereof.

It will be understood that certain compounds of the present invention may exist in solvated, for example hydrated, as well as unsolvated forms. It is to be understood that the present invention encompasses all such solvated forms.

### DETAILED DESCRIPTION

In an aspect, the invention comprises a subset of the compounds of formula I, as defined by formula IA,

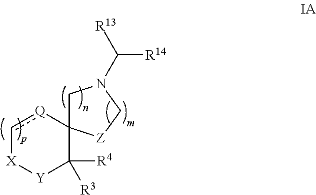

wherein:
Q is selected from CR⁷R⁸, C═O, S(O)_{q} and O;
X and Y are selected from CR¹¹R¹², NH and N-alkyl;
   wherein:
      X is CR¹¹R¹² when Y is NH or N-alkyl,
      Y is CR¹¹R¹² when X is NH or N-alkyl;
      X is CR¹¹R¹² when p is 0 and Q is S(O)_{q} or O;
      wherein Q is CR⁷R⁸ when p is 0 and X is NH or N-alkyl; and
      one of X and Y is NH or N-alkyl;
Z is selected from CH₂ and O; wherein Z is CH₂ when Q is O or S(O)_{q};
R³ and R⁴ are H; or R³ and R⁴ may both be O, wherein said O atoms are linked by an ethylene group to form an ethylenedioxy group;
R⁷ is selected from H, F, Cl, OH and alkoxy;
R⁸ is absent or is selected from H, F, Cl, OH and alkoxy;
   or R⁷ and R⁸ may both be O, wherein said O atoms are linked by an ethylene group to form an ethylenedioxy group;
R¹³ is phenyl substituted with 1, 2 or 3 substituents selected from F and Cl;
R¹⁴ is phenyl substituted with 1, 2 or 3 substituents selected from F and Cl;
R¹⁵ and R¹⁶ are independently selected from H and alkyl;
n is 1 or 2, wherein n is 1 when m is 2;
m is 1 or 2, wherein m is 1 when n is 2;
p is 0 or 1; wherein p is 1 when n is 2
q is 0, 1 or 2;
- - - - is absent or represents a bond; wherein when - - - - is a bond R² is absent, Q is CR⁷R⁸, R⁸ is absent, and p is 1;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1, 2, 3, 4 or 5 substituents independently selected from cycloalkyl, alkoxy, OH, —CN, CF₃, COOR¹⁵, CONR¹⁵R¹⁶, F, Cl and NR¹⁵R¹⁶;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1, 2, 3, 4 or 5 substituents independently selected from alkyl, OH, —CN, CF₃, COOR¹⁵, CONR¹⁵R¹⁶, F, Cl and NR¹⁵R¹⁶;
and tautomers, stereoisomers (including enantiomers, diastereoisomers and racemic and scalemic mixtures thereof), pharmaceutically acceptable salts and solvates thereof.

In an aspect, the invention comprises a subset of the compounds of formula I, as defined by formula IB,

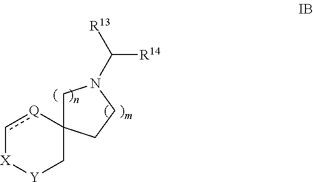

wherein
Q is selected from CR⁷R⁸, C═O, S(O)_{q} and O;
X and Y are selected from CH₂, NH and N-methyl;
   wherein:
      one of X and Y is NH or N-methyl;
      X is CH₂ when Y is NH or N-methyl; and
      Y is CH₂ when X is NH or N-methyl;
R⁷ is selected from H, F, Cl, OH, alkoxy;
R⁸ is absent or is selected from H, F and Cl; or R⁷ and R⁸ may both be O, wherein said O atoms are linked by an ethylene group to form a straight chain or branched ethylenedioxy group;
R¹³ is phenyl substituted with 1 substituent selected from F and Cl;
R¹⁴ is phenyl substituted with 1 substituent selected from F and Cl;
R¹⁵ and R¹⁶ are independently selected from H and alkyl;
n is 1 or 2, wherein n is 1 when m is 2;
m is 1 or 2, wherein m is 1 when n is 2;
- - - - is absent or represents a bond; wherein when - - - - is a bond R² is absent, Q is CR⁷R⁸, R⁸ is absent, and p is 1; q is 0, 1 or 2;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1, 2, 3, 4 or 5 substituents independently selected from cycloalkyl, alkoxy, OH, —CN, CF₃, COOR¹⁵, CONR¹⁵R¹⁶, F, Cl and NR¹⁵R¹⁶;
   alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1, 2, 3, 4 or 5 substituents independently selected from alkyl, OH, —CN, CF₃, COOR¹⁵, CONR¹⁵R¹⁶, F, Cl and NR¹⁵R¹⁶;
and tautomers, stereoisomers (including enantiomers, diastereoisomers and racemic and scalemic mixtures thereof), pharmaceutically acceptable salts and solvates thereof.

In an aspect, the invention comprises a subset of the compounds of formula I, as defined by formula IC,

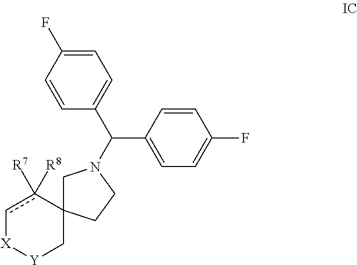

wherein:
X and Y are selected from CH₂ and NH;
   wherein:
      one of X and Y is NH,
      X is CH₂ when Y is NH; and
      Y is CH₂ when X is NH;
R⁷ is selected from H, F and OH;
R⁸ is absent or is selected from H and F; or R⁷ and R⁸ may both be O, wherein said O atoms are linked by an alkylene group to form an ethylenedioxy group;
- - - - is absent or represents a bond; wherein when - - - - is a bond, R⁸ is absent;
   and tautomers, stereoisomers (including enantiomers, diastereoisomers and racemic and scalemic mixtures thereof), pharmaceutically acceptable salts and solvates thereof.

The present invention also comprises the following aspects and combinations thereof.

In an aspect X and Y are selected from CH₂, NH and N-methyl.

In an aspect X and Y are selected from CH₂ and NH.

In an aspect X is CH₂ and Y is NH.

In an aspect Q is selected from CR⁷R⁸, C═O, C═N—OH, C═N—O-alkyl, NH, N-alkyl, S(O)_{q} and O.

In an aspect Q is selected from CR⁷R⁸, C═O, O and S(O)_{q}.

In an aspect, Q is selected from C═O, O, S, SO₂ and CR⁷R⁸.

In an aspect Q is selected from CR⁷R⁸, C═O, O and S.

In an aspect Q is selected from CR⁷R⁸, C═O and O.

In an aspect Q is selected from CR⁷R⁸ and C═O.

In an aspect Q is CR⁷R⁸.

In an aspect Z is CH₂ or O.

In an aspect Z is CH₂.

In an aspect R³ and R⁴ are independently selected from H, OH, alkoxy and alkyl;
or R³ and R⁴ may both be O, wherein said O atoms are linked by an alkylene group to form a straight chain or branched alkylenedioxy group.

In an aspect R³ and R⁴ are H; or R³ and R⁴ may both be O, wherein said O atoms are linked by an alkylene group to form a straight chain or branched alkylenedioxy group.

In an aspect R³ and R⁴ are H; or R³ and R⁴ may both be O, wherein said O atoms are linked by an ethylene group to form an ethylenedioxy group.

In an aspect R³ and R⁴ are H.

In an aspect R¹³ is phenyl substituted with 1, 2 or 3 substituents selected from F and Cl.

In an aspect R¹³ is phenyl substituted with 1 substituent selected from F and Cl.

In an aspect R¹³ is phenyl substituted with 1 F substituent.

In an aspect R¹³ is phenyl substituted in the para position with 1 substituent selected from F and Cl.

In an aspect R¹³ is para-fluoro-phenyl.

In an aspect R¹⁴ is phenyl substituted with 1, 2 or 3 substituents independently selected from alkyl, cycloalkyl, alkoxy, S-alkyl, OH, F, Cl, Br, I, —CN, OCF₃, CF₃ and NR¹⁵R¹⁶.

In an aspect R¹⁴ is phenyl substituted with 1, 2 or 3 substituents selected from F and Cl.

In an aspect R¹⁴ is phenyl substituted with 1 substituent selected from F and Cl.

In an aspect R¹⁴ is phenyl substituted with 1 F substituent.

In an aspect R¹⁴ is phenyl substituted in the para position with 1 substituent selected from F and Cl.

In an aspect R¹⁴ is para-fluoro-phenyl.

In an aspect R¹³ and R¹⁴ are both para-fluoro-phenyl.

In an aspect R⁷ is selected from H, F, Cl and OH, and R⁸ is absent or is selected from H, F, Cl and OH; or R⁷ and R⁸ may both be O, wherein said O atoms are linked by an alkylene group to form an alkylenedioxy group.

In an aspect R⁷ is selected from H, F and OH and R⁸ is absent or is selected from H, F and OH; or R⁷ and R⁸ may both be O, wherein said O atoms are linked by an ethylene group to form an ethylenedioxy group.

In an aspect q is 0.

In an aspect n is 1 or 2, wherein n is 1 when m is 2; and m is 1 or 2, wherein m is 1 when n is 2.

In an aspect m is 1.

In an aspect n is 1.

In an aspect m is 1 and n is 1.

p is 0 or 1; wherein p is 1 when n is 2;

In an aspect p is 1.

In an aspect m is 1, n is 1 and p is 1.

In an aspect, the invention comprises a compound of formula I selected from:

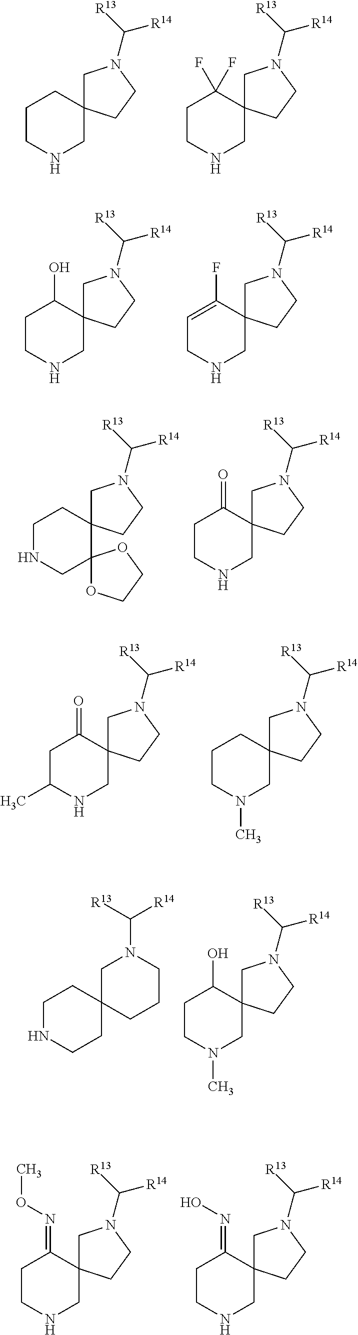

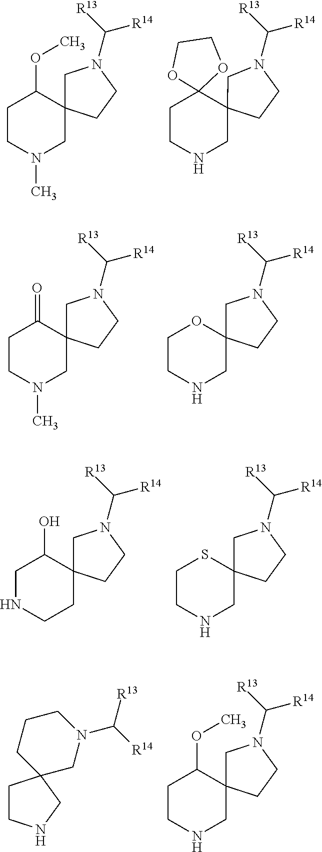

and pharmaceutically acceptable salts and solvates thereof.

In an aspect, the invention comprises a compound of formula I selected from:
2-[bis(4-fluorophenyl)methyl]-2,7-diazaspiro[4.5]decan-10-one;
2-[bis(4-fluorophenyl)methyl]-7-methyl-2,7-diazaspiro[4.5]decan-10-one;
2-[bis(4-fluorophenyl)methyl]-2,7-diazaspiro[4.5]decan-10-ol;
2-[bis(4-fluorophenyl)methyl]-7-methyl-2,7-diazaspiro[4.5]decan-10-ol;
2-[bis(4-fluorophenyl)methyl]-10-methoxy-2,7-diazaspiro[4.5]decane;
8-[bis(4-fluorophenyl)methyl]-1,4-dioxa-8,12-diazadispiro[4.0.4⁶.4⁵]tetradecane;
(5R)-2-[bis(4-fluorophenyl)methyl]-2,7-diazaspiro[4.5]decan-10-one;
(5S)-2-[bis(4-fluorophenyl)methyl]-2,7-diazaspiro[4.5]decan-10-one;
2-[bis(4-fluorophenyl)methyl]-2,7-diazaspiro[4.5]decan-10-ol;
2-[bis(4-fluorophenyl)methyl]-N-methoxy-2,7-diazaspiro[4.5]decan-10-imine;
(5R,10E)-2-[bis(4-fluorophenyl)methyl]-N-methoxy-2,7-diazaspiro[4.5]decan-10-imine;
(5S,10E)-2-[bis(4-fluorophenyl)methyl]-N-methoxy-2,7-diazaspiro[4.5]decan-10-imine;
N-[2-[bis(4-fluorophenyl)methyl]-2,7-diazaspiro[4.5]decan-10-ylidene]hydroxylamine
N-[(5R,10E)-2-[bis(4-fluorophenyl)methyl]-2,7-diazaspiro[4.5]decan-10-ylidene]hydroxylamine;
N-[(5S,10E)-2-[bis(4-fluorophenyl)methyl]-2,7-diazaspiro[4.5]decan-10-ylidene]hydroxylamine;
2-[bis(4-fluorophenyl)methyl]-10,10-difluoro-2,7-diazaspiro[4.5]decane;
(5R)-2-[bis(4-fluorophenyl)methyl]-10,10-difluoro-2,7-diazaspiro[4.5]decane;
(5S)-2-[bis(4-fluorophenyl)methyl)methyl]-10,10-difluoro-2,7-diazaspiro[4.5]decane;
2-[bis(4-fluorophenyl)methyl]-10-fluoro-2,7-diazaspiro[4.5]dec-9-ene;
(5R)-2-[bis(4-fluorophenyl)methyl]-10-fluoro-2,7-diazaspiro[4.5]dec-9-ene;
(5S)-2-[bis(4-fluorophenyl)methyl]-10-fluoro-2,7-diazaspiro[4.5]dec-9-ene;
2-[bis(4-chlorophenyl)methyl]-2,7-diazaspiro[4.5]decan-10-one;
8-[bis(4-fluorophenyl)methyl]-1,4-dioxa-8,13-diazadispiro[4.0.4⁶.4⁵]tetradecane;
2-[bis(4-fluorophenyl)methyl]-2,8-diazaspiro[4.5]decan-6-ol;
2-[bis(4-fluorophenyl)methyl]-2,7-diazaspiro[4.5]decane;
2-[bis(4-fluorophenyl)methyl]-7-methyl-2,7-diazaspiro[4.5]decane;
7-[bis(4-fluorophenyl)methyl]-2,7-diazaspiro[4.5]decane;
2-[bis(4-fluorophenyl)methyl]-2,9-diazaspiro[5.5]undecane;
9-[bis(4-fluorophenyl)methyl]-2,9-diazaspiro[5.5]undecane;
2-[bis(4-fluorophenyl)methyl]-2,8-diazaspiro[4.5]decane;
2-[bis(4-chlorophenyl)methyl]-2,8-diazaspiro[4.5]decane;
2-[bis(4-fluorophenyl)methyl]-6-oxa-2,9-diazaspiro[4.5]decane;
2-[bis(4-chlorophenyl)methyl]-6-oxa-2,9-diazaspiro[4.5]decane;
9-[bis(4-fluorophenyl)methyl]-1-oxa-4,9-diazaspiro[5.5]undecane;
9-[bis(4-fluorophenyl)methyl]-1-thia-4,9-diazaspiro[5.5]undecane;
   and pharmaceutically acceptable salts and solvates thereof.

Preferably, the invention comprises a compound of formula I selected from:
2-[bis(4-fluorophenyl)methyl]-2,7-diazaspiro[4.5]decan-10-ol;
2-[bis(4-fluorophenyl)methyl]-10,10-difluoro-2,7-diazaspiro[4.5]decane;
(5R)-2-[bis(4-fluorophenyl)methyl]-10,10-difluoro-2,7-diazaspiro[4.5]decane;
(5S)-2-[bis(4-fluorophenyl)methyl]-10, 10-difluoro-2,7-diazaspiro[4.5]decane;
2-[bis(4-fluorophenyl)methyl]-10-fluoro-2,7-diazaspiro[4.5]dec-9-ene;
(5R)-2-[bis(4-fluorophenyl)methyl]-10-fluoro-2,7-diazaspiro[4.5]dec-9-ene;
(5S)-2-[bis(4-fluorophenyl)methyl]-10-fluoro-2,7-diazaspiro[4.5]dec-9-ene;
8-[bis(4-fluorophenyl)methyl]-1,4-dioxa-8,13-diazadispiro[4.0.4⁶.4⁵]tetradecane;
2-[bis(4-fluorophenyl)methyl]-2,7-diazaspiro[4.5]decane;
   and pharmaceutically acceptable salts and solvates thereof.

Therapeutic Applications

As previously mentioned, the compounds of the present invention are potent inhibitors of dopamine transporters. They are therefore useful in the treatment of disease conditions for which over-activity of a dopamine transporter is a causative factor.

The compounds of the present invention are preferably selective for dopamine transporters over noradrenaline and serotonin transporters. In the present context, the word “selective” means the compound has an IC50 value that is at least 10-fold selective for the dopamine transporter than for each of the noradrenaline and serotonin transporters, preferably at least 20-fold, more preferably at least 30-fold, even more preferably 50-fold, most preferably 100-fold higher for the dopamine transporter than for each of the noradrenaline and serotonin transporters.

Accordingly, the present invention provides a compound of formula I for use in therapy.

The present invention also provides for the use of a compound of formula I in the manufacture of a medicament for the treatment or prevention of a condition, disease or disorder ameliorated by inhibition of a dopamine transporter.

The present invention also provides a compound of formula I for use in the treatment or prevention of a condition, disease or disorder ameliorated by inhibition of a dopamine transporter.

The present invention also provides a method of treatment of a condition, disease or disorder ameliorated by inhibition of a dopamine transporter comprising administration to a subject in need thereof a therapeutically effective amount of a compound of formula I.

In one aspect, the condition, disease or disorder ameliorated by inhibition of a dopamine transporter includes sexual dysfunction, affective disorders, anxiety, depression, Tourette syndrome, Angelman syndrome, attention deficit disorder (ADD), attention deficit hyperactivity disorder (ADHD), obesity, pain, obsessive-compulsive disorder, movement disorders, CNS disorders, sleep disorders, narcolepsy, conduct disorder, substance abuse (including smoking cessation), eating disorders, chronic fatigue and impulse control disorders.

In a particular aspect, the condition, disease or disorder is selected from ADD, ADHD and binge eating disorder.

In the context of the present invention, references herein to “treatment” include references to curative, palliative and prophylactic treatment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Combination Therapy

When combination therapy is employed, the compounds of the present invention and said combination agents may exist in the same or different pharmaceutical compositions, and may be administered separately, sequentially or simultaneously.

The compounds of the invention may be administered as a combination with at least one other active pharmaceutical ingredient for the treatment of mood disorders, disorders such as depression, refractory depression, bipolar depression, and psychotic depression. Such a pharmaceutical combination may be in the form of a unit dosage form or it may be in the form of a package comprising the at least two active components separately. In a further aspect, the invention relates to such pharmaceutical combinations. In a further aspect, the invention therefore relates to a pharmaceutical combination comprising a therapeutically effective amount of an compound of the invention and a second active substance, for simultaneous or sequential administration.

In an aspect the invention relates to a compound of the invention in combination with another therapeutic agent wherein the other therapeutic agent is selected from:

a tricyclic antidepressant (Amitriptyline, Clomipramine, Doxepin, Imipramine, Trimipramine Desipramine, Nortriptyline, Protriptyline),

tetracyclic antidepressant (Amoxapine, Maprotiline, Mazindol, Mianserin, Mirtazapine, Setiptiline), selective serotonin reuptake inhibitor (Citalopram, Escitalopram, Paroxetine, Fluoxetine, Fluvoxamine, Sertraline),

serotonin antagonist and reuptake inhibitors (Etoperidone, Nefazodone, Trazodone), selective norepinephrine reuptake inhibitor (Atomoxetine, Reboxetine, Viloxazine),

serotonin and norepinephrine reuptake inhibitor (Desvenlafaxine, Duloxetine, Milnacipran, Venlafaxine),

monoamine oxidase inhibitor (Isocarboxazid, Phenelzine, Selegiline, Tranylcypromine, Moclobemide, Pirlindole),

mood stabilisers (Lithium, Valproic Acid, Lamotrigine, Carbamazepine, Oxcarbazepine) and/or antipsychotics (Clozapine, Olanzapine, Risperidone, Quetiapine, Ziprasidone, Amisulpride, Asenapine, Paliperidone, Iloperidone, Zotepine, Sertindole, Lurasidone, Aripiprazole, Haloperidol, Droperidol, Chlorpromazine, Fluphenazine Perphenazine, Prochlorperazine, Thioridazine, Trifluoperazine, Mesoridazine, Periciazine, Promazine, Triflupromazine, Levomepromazine, Promethazine, Pimozide, Cyamemazine, Chlorprothixene, Clopenthixol, Flupenthixol, Thiothixene, Zuclopenthixol).

In addition to treating the primary disease symptoms or the therapeutic lag phase, DAT inhibitors may be used adjunctively to treat medication induced sedation, common in diseases such as bipolar depression as well as sexual dysfunction which is a common side effect of antidepressant treatment, particularly SSRIs.

The compounds of the invention may be administered as a combination with at least one other active pharmaceutical ingredient for the treatment of smoking cessation and mitigation of nicotine withdrawal and weight gain. Such a pharmaceutical combination may be in the form of a unit dosage form or it may be in the form of a package comprising the at least two active components separately. In a further aspect, the invention relates to such pharmaceutical combinations.

In a further aspect, the invention therefore relates to a pharmaceutical combination comprising a therapeutically effective amount of an compound of the invention and a second active substance, for simultaneous or sequential administration.

In an aspect, the invention relates to a compound of the invention in combination with another therapeutic agent wherein the other therapeutic agent is selected from:

Nicotine replacement therapies (nicotine patches, nicotine gum, nicotine sprays, nicotine sublingual tablets, nicotine lozenges and nicotine inhalers), nicotinic full/partial agonists (Nicotine, Varenicline, Lobeline), opioid antagonists/inverse agonists (Naloxone, Naltrexone, Buprenorphine).

The compounds of the invention may be administered as a combination with at least one other active pharmaceutical ingredient for the treatment of ADHD. Such a pharmaceutical combination may be in the form of a unit dosage form or it may be in the form of a package comprising the at least two active components separately. In a further aspect, the invention relates to such pharmaceutical combinations.

In a further aspect, the invention therefore relates to a pharmaceutical combination comprising a therapeutically effective amount of an compound of the invention and a second active substance, for simultaneous or sequential administration.

In an aspect, the invention relates to a compound of the invention in combination with another therapeutic agent wherein the other therapeutic agent is selected from:

Norepinephrine reuptake inhibitors (Atomoxetine, Reboxetine, Viloxazine), alpha-adrenoceptor agonists (Guanfacine, Clonidine).

The compounds of the invention may be administered as a combination with at least one other active pharmaceutical ingredient for the treatment of movement disorders such as Parkinson's disease and Restless Leg Syndrome. Such a pharmaceutical combination may be in the form of a unit dosage form or it may be in the form of a package comprising the at least two active components separately. In a further aspect, the invention relates to such pharmaceutical combinations.

In a further aspect, the invention therefore relates to a pharmaceutical combination comprising a therapeutically effective amount of an compound of the invention and a second active substance, for simultaneous or sequential administration.

In an aspect, the invention relates to a compound of the invention in combination with another therapeutic agent wherein the other therapeutic agent is selected from:

A dopamine precursor (L-dopa) a dopaminergic agent (Levodopa-carbidopa, Levodopa-benzerazide), a dopaminergic and anti-cholinergic agent (amantadine), an anti-cholinergic agent (trihexyphenidyl, benztropine, ethoproprazine, or procyclidine), a dopamine agonist (apomorphine, bromocriptine, cabergoline, lisuride, pergolide, pramipexole, or ropinirole), a MAO-B (monoamine oxidase B) inhibitor (selegiline, rasageline or deprenyl0, a COMT (catechol O-methyltransferase) inhibitor (tolcapone or entacapone.

### Definitions

“Alkyl” is as defined above and includes saturated hydrocarbon residues including:
linear groups of up to 6 carbon atoms (C₁-C₆), or of up to 4 carbon atoms (C₁-C₄). Examples of such alkyl groups include, but are not limited, to C₁-methyl, C₂-ethyl, C₃-propyl and C₄-n-butyl.
branched groups of between 3 and 6 carbon atoms (C₃-C₆), or of up to 4 carbon atoms (C₃-C₄). Examples of such alkyl groups include, but are not limited to, C₃-iso-propyl, C₄-sec-butyl, C₄-iso-butyl, C₄-tert-butyl and C₅-neo-pentyl.
   each optionally substituted as stated above.

“Cycloalkyl” is as defined above and includes monocyclic saturated hydrocarbon of between 3 and 7 carbon atoms, or from 3 to 6 carbon atoms, or from 3 to 5 carbon atoms, or from 3 to 4 carbon atoms. Examples of suitable monocyclic cycloalkyl groups include cyclopropyl, cyclobutyl, cyclopentyl, cyclohexyl and cycloheptyl. Cycloalkyl is optionally substituted as stated above.

“Alkylene” is a bivalent C₁₋₃ straight-chained alkyl radical, such as —(CH₂)—, —(CH₂)₂—, —(CH₂)₃— or a bivalent C₃₋₄ branched alkyl radical such as —CH(CH₃)CH, CH₂CH(CH₃)—, —CH(CH₃)CH(CH₃)—. Alkylene is optionally substituted as stated above.

“Alkoxy” is as defined above and includes O-linked hydrocarbon residues including:
linear groups of between 1 and 6 carbon atoms (C₁-C₆), or of between 1 and 4 carbon atoms (C₁-C₄). Examples of such alkoxy groups include, but are not limited to, C₁-methoxy, C₂-ethoxy, C₃-n-propoxy and C₄-n-butoxy.
branched groups of between 3 and 6 carbon atoms (C₃-C₆) or of between 3 and 4 carbon atoms (C₃-C₄). Examples of such alkoxy groups include, but are not limited to, C₃-iso-propoxy, and C₄-sec-butoxy and tert-butoxy.
   each optionally substituted as stated above.

“Heterocyclyl” is defined above. Examples of suitable heterocyclyl groups include aziridinyl, azetidinyl, pyrrolidinyl, imidazolidinyl, imidazolyl, morpholine, thiomorpholine pyrazolidinyl, piperidinyl and piperazinyl (optionally substituted as stated above).

The term “O-linked”, such as in “O-linked hydrocarbon residue”, means that the hydrocarbon residue is joined to the remainder of the molecule via an oxygen atom.

In groups such as —CN and —CH₂CH(CH₃)—, “—” denotes the point of attachment of the substituent group to the remainder of the molecule.

“Pharmaceutically acceptable salt” means a physiologically or toxicologically tolerable salt and includes, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that can be formed include sodium, potassium, calcium, magnesium and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that can be formed include hydrochlorides, hydrobromides, sulfates, phosphates, acetates, citrates, lactates, tartrates, mesylates, succinates, oxalates, phosphates, esylates, tosylates, benzenesulfonates, naphthalenedisulphonates, maleates, adipates, fumarates, hippurates, camphorates, xinafoates, p-acetamidobenzoates, dihydroxybenzoates, hydroxynaphthoates, succinates, ascorbates, oleates, bisulfates and the like.

Hemisalts of acids and bases can also be formed, for example, hemisulfate and hemicalcium salts.

For a review of suitable salts, see “Handbook of Pharmaceutical Salts: Properties, Selection and Use” by Stahl and Wermuth (Wiley-VCH, Weinheim, Germany, 2002).

“Prodrug” refers to a compound which is convertible in vivo by metabolic means (e.g. by hydrolysis, reduction or oxidation) to a compound of the invention. Suitable groups for forming pro-drugs are described in ‘The Practice of Medicinal Chemistry, 2^{nd} Ed. pp 561-585 (2003) and in F. J. Leinweber, Drug Metab. Res., 1987, 18, 379.

The compounds of the invention can exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

Compounds of the invention may exist in one or more geometrical, optical, enantiomeric, diastereomeric, conformational and tautomeric forms, including but not limited to cis- and trans-forms, E- and Z-forms, R-, S- and meso-forms, keto- and enol-forms, and conformers. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods (e.g. asymmetric synthesis).

An example of a compound of the invention that exhibits diastereoisomerism is 2-[bis(4-fluorophenyl)methyl]-2,7-diazaspiro[4.5]decan-10-ol. The present invention therefore encompasses all diasteromeric forms of this compound, as illustrated below.

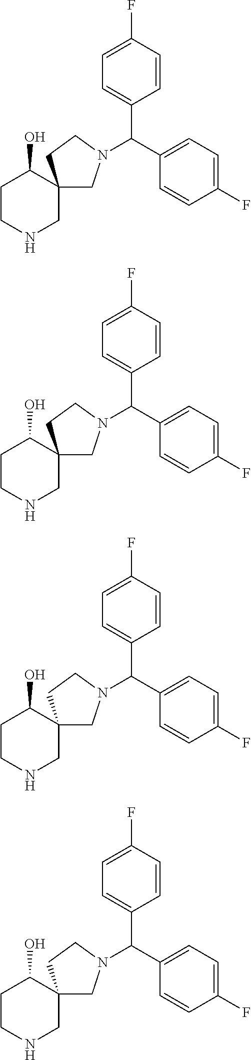 [SMILES: O[C@@H]1CCNC[C@]12CCN(C(c1ccc(F)cc1)c1ccc(F)cc1)C2 ; O[C@H]1CCNC[C@]12CCN(C(c1ccc(F)cc1)c1ccc(F)cc1)C2 ; O[C@@H]1CCNC[C@@]12CCN(C(c1ccc(F)cc1)c1ccc(F)cc1)C2 ; O[C@H]1CCNC[C@@]12CCN(C(c1ccc(F)cc1)c1ccc(F)cc1)C2]

Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

General Methods

The compounds of formula I should be assessed for their biopharmaceutical properties, such as solubility and solution stability (across pH), permeability, etc., in order to select the most appropriate dosage form and route of administration for treatment of the proposed indication. They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention which may impart either a functional (i.e., drug release rate controlling) and/or a non-functional (i.e., processing aid or diluent) characteristic to the formulations. The choice of excipient will to a large extent depend on factors such as the particular mode of administration, the effect of the excipient on solubility and stability, and the nature of the dosage form.

Compounds of the invention intended for pharmaceutical use may be administered as a solid or liquid, such as a tablet, capsule or solution. Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly, the present invention provides a pharmaceutical composition comprising a compound of formula I and a pharmaceutically acceptable carrier, diluent or excipient.

The compounds of the invention may also be administered directly into the blood stream, into subcutaneous tissue, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous or oily solutions. Where the solution is aqueous, excipients such as sugars (including but not restricted to glucose, manitol, sorbitol, etc.), salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

Parenteral formulations may include implants derived from degradable polymers such as polyesters (i.e., polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds of formula I used in the preparation of parenteral solutions may be increased by the use of appropriate formulation techniques, such as the incorporation of co-solvents and/or solubility-enhancing agents such as surfactants, micelle structures and cyclodextrins.

In one aspect, the compounds of the invention may be administered orally. Oral administration may involve swallowing, so that the compound enters the gastrointestinal tract, and/or buccal, lingual, or sublingual administration by which the compound enters the blood stream directly from the mouth. Formulations suitable for oral administration include solid plugs, solid microparticulates, semi-solid and liquid (including multiple phases or dispersed systems) such as tablets; soft or hard capsules containing multi- or nano-particulates, liquids, emulsions or powders; lozenges (including liquid-filled); chews; gels; fast dispersing dosage forms; films; ovules; sprays; and buccal/mucoadhesive patches.

Formulations suitable for oral administration may also be designed to deliver the compounds of the invention in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 2001, 11 (6), 981-986.

The formulation of tablets is discussed in Pharmaceutical Dosage Forms: Tablets, Vol. 1, by H. Lieberman and L. Lachman (Marcel Dekker, New York, 1980).

For administration to human patients, the total daily dose of the compounds of the invention is typically in the range 0.01 mg and 1000 mg, or between 0.1 mg and 250 mg, or between 1 mg and 50 mg depending, of course, on the mode of administration.

The total dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

Synthetic Methods

The compounds of the present invention can be prepared according to the procedures of the following schemes and examples, using appropriate materials, and are further exemplified by the specific examples provided herein below. Moreover, by utilising the procedures described herein, one of ordinary skill in the art can readily prepare additional compounds that fall within the scope of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein above.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 4^{th} Edition, 2006, may be used. For example, a common amino protecting group suitable for use herein is tert-butoxy carbonyl (Boc), which is readily removed by treatment with an acid such as trifluoroacetic acid or hydrogen chloride in an organic solvent such as dichloromethane. Alternatively the amino protecting group may be a benzyloxycarbonyl (Z) group which can be removed by hydrogenation with a palladium catalyst under a hydrogen atmosphere or 9-fluorenylmethyloxycarbonyl (Fmoc) group which can be removed by solutions of secondary organic amines such as diethylamine or piperidine in an organic solvent. Carboxyl groups are typically protected as esters such as methyl, ethyl, benzyl or tert-butyl which can all be removed by hydrolysis in the presence of bases such as lithium or sodium hydroxide. Benzyl protecting groups can also be removed by hydrogenation with a palladium catalyst under a hydrogen atmosphere whilst tert-butyl groups can also be removed by trifluoroacetic acid. Alternatively a trichloroethyl ester protecting group is removed with zinc in acetic acid. A common hydroxy protecting group suitable for use herein is a methyl ether, deprotection conditions comprise refluxing in 48% aqueous HBr for 1-24 hours, or by stirring with borane tribromide in dichloromethane for 1-24 hours. Alternatively where a hydroxy group is protected as a benzyl ether, deprotection conditions comprise hydrogenation with a palladium catalyst under a hydrogen atmosphere.

The compounds according to general formula I can be prepared using conventional synthetic methods for example, but not limited to, the routes outlined in the schemes below.

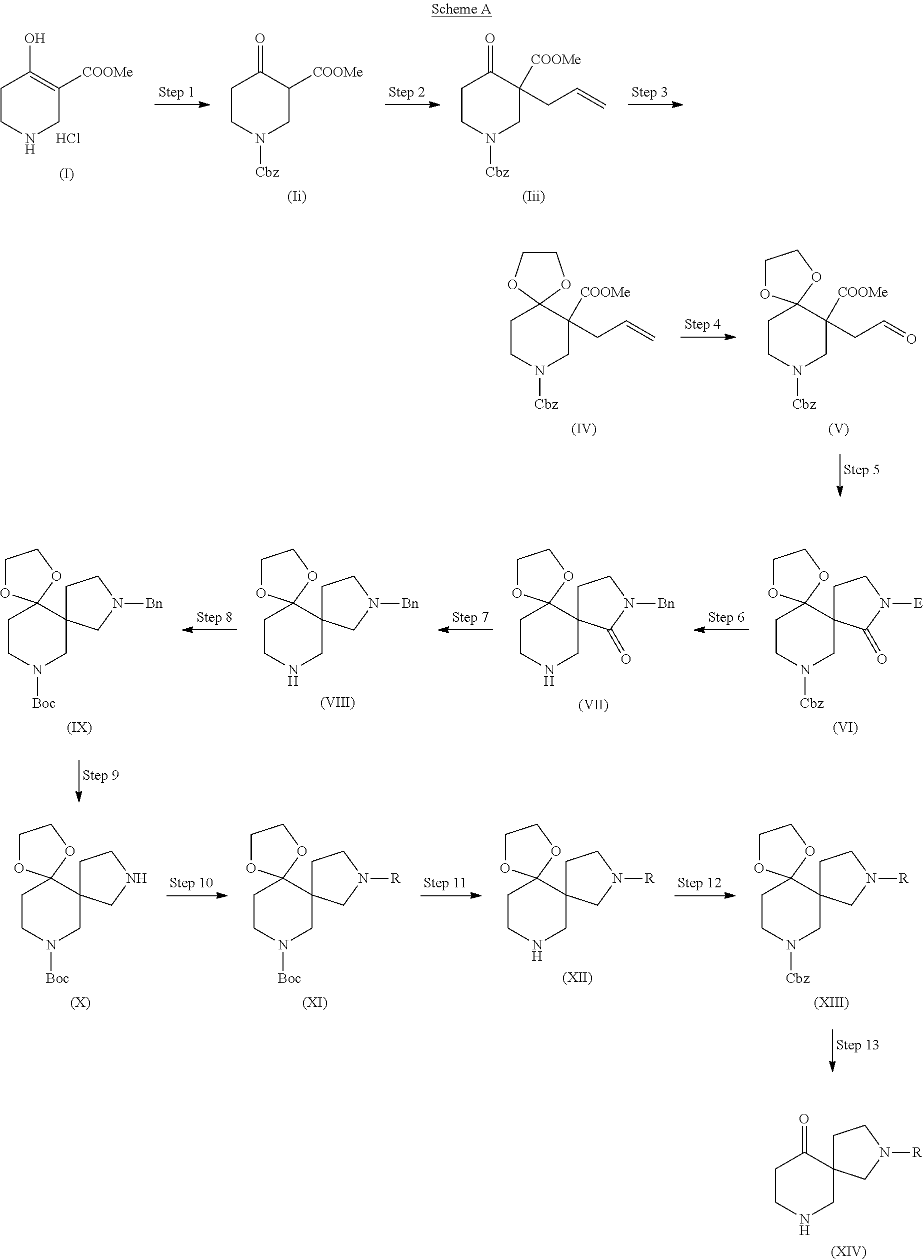

Step 1

Compound of formula II may be obtained by N-protection of compound I (commercially available from Sigma-Aldrich) under standard literature conditions such as by reaction with benzyl chloroformate, with the presence of a suitable base such as triethylamine, carrying out the reaction in a suitable solvent, e.g. DCM, typically at room temperature. The reaction takes about 12 hours to complete.

Step 2

Compound of formula III may be obtained by alkylation of compound II with allyl bromide, after deprotonation using a suitable base, such as NaH, in a suitable solvent, e. g. DMF, carrying out the reaction at a temperature between 0° C. and room temperature. The reaction takes about 4 hours to complete.

Step 3

Compound of formula IV may be obtained by ketone protection of compound III by reaction with ethylene glycol, in presence of catalytic amount of p-Toluensulfonic in a suitable solvent, such as toluene, using Dean Stark apparatus, typically at reflux temperature. The reaction takes about 16 hours to complete.

Step 4

Compound of formula V may be obtained by oxidation of compound IV using an aqueous solution of OsO₄ in a mixture of THF/water, in presence of NaIO₄, carrying out the reaction typically at room temperature. The reaction takes about 1 hour to complete.

Step 5

Compound of formula VI may be obtained by reductive amination of compound V with a suitable primary amine, such as benzylamine, in a suitable solvent, such as THF, in presence of a reducing agent like Na(AcO)₃BH, followed by spontaneous lactam ring closure. The reaction is carried out typically at room temperature and takes about 12 hours to complete.

Step 6

Compound of formula VII can be obtained by N-deprotection of compound VI by hydrogenolysis such as hydrogenation over palladium catalyst on carbon, and the like, in a suitable solvent, e. g. MeOH at a temperature of about 25° C., over a period of about 0.5 hour.

Step 7

Compound of formula VIII may be obtained by reduction of compound VII using a suitable reducing agent, e. g. LiAlH₄, carrying out the reaction in a suitable solvent, such as THF at elevate temperature (preferably around 65° C.). The reaction takes about 4 hours to complete.

Step 8

Compound of formula IX may be obtained by N-protection of compound VIII under standard literature conditions such as by reaction with Di-tert-butyl dicarbonate in a mixture of THF/water, in presence of a suitable base, such as Na₂CO₃, at a temperature around 0° C. The reaction takes about 1 hour to complete.

Step 9

Compound of formula X may be obtained from compound IX by removing the benzyl group by hydrogenolysis, e. g. using ammonium formate and palladium on carbon, in a suitable solvent such as methanol under reflux. The reaction takes about 1 hour.

Step 10

Compound XI may be obtained from compound X by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 2 hours to complete.

Step 11

Compound XII can be obtained from compound XI by removing the Boc group under acidic conditions, e. g. TFA in dichloromethane solution, typically at room temperature. The reaction takes about 1 hour.

Step 12

Compound of formula XIII may be obtained by N-protection of compound XII under standard literature conditions such as by reaction with benzyl chloroformate, with the presence of a suitable base such as triethylamine, carrying out the reaction in a suitable solvent, e.g. DCM, typically at room temperature.

The reaction takes about 1 hour to complete.

Step 13

Compound XIV can be obtained from compound XIII by ketale cleavage under acidic conditions, e. g. HClO₄ in dichloromethane solution, typically at room temperature. The reaction takes about 3 hours to complete.

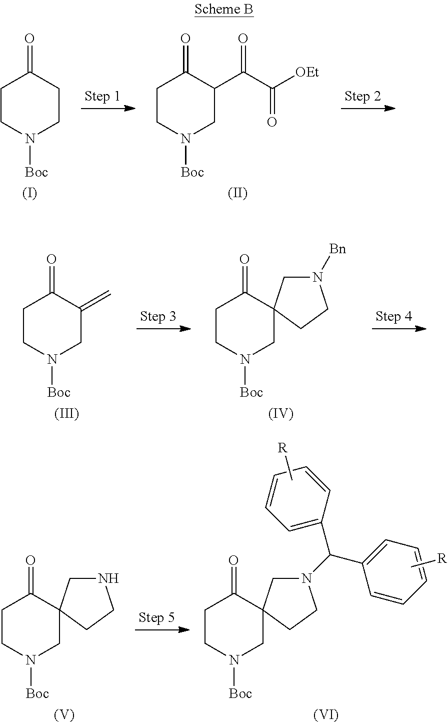

Step 1

Compound of formula II can be prepared from compound of formula I (commercially available from Sigma-Aldrich) by reaction with diethyl oxalate in presence of a suitable base, such as LiOEt or LiHMDS, in a suitable solvent such as EtOH or Et₂O, at a temperature between −78° C. and room temperature. The reaction takes about 12 hours to complete.

Step 2

Compound of formula III may be prepared from compound of formula II by reaction with formaldehyde in presence of a suitable base, such as NaOH, in a mixture of THH/water. The reaction proceeds typically at room temperature and takes about 20 minute to complete.

Step 3

Compound of formula IV may be prepared from compound of formula III by [3+2] cycloaddition with N-(Methoxymethyl)-N-(trimethylsilylmethyl)benzylamine in presence of TFA, in a suitable solvent, e. g. dichloromethane, keeping the temperature below 5° C. during the addiction. The reaction proceeds at room temperature and takes 12 hours.

Step 4

Compound of formula V may be obtained by removing the benzyl group treating compound IV with 1-chloroethyl chloroformate in a suitable solvent, such as dichloromethane, in presence of a suitable base, e. g. diisopropylamine, typically at reflux temperature for about 2 hours, followed by reflux in MeOH for about 1 hour.

Step 5

Compound VI may be obtained from compound V by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 3 hours to complete.

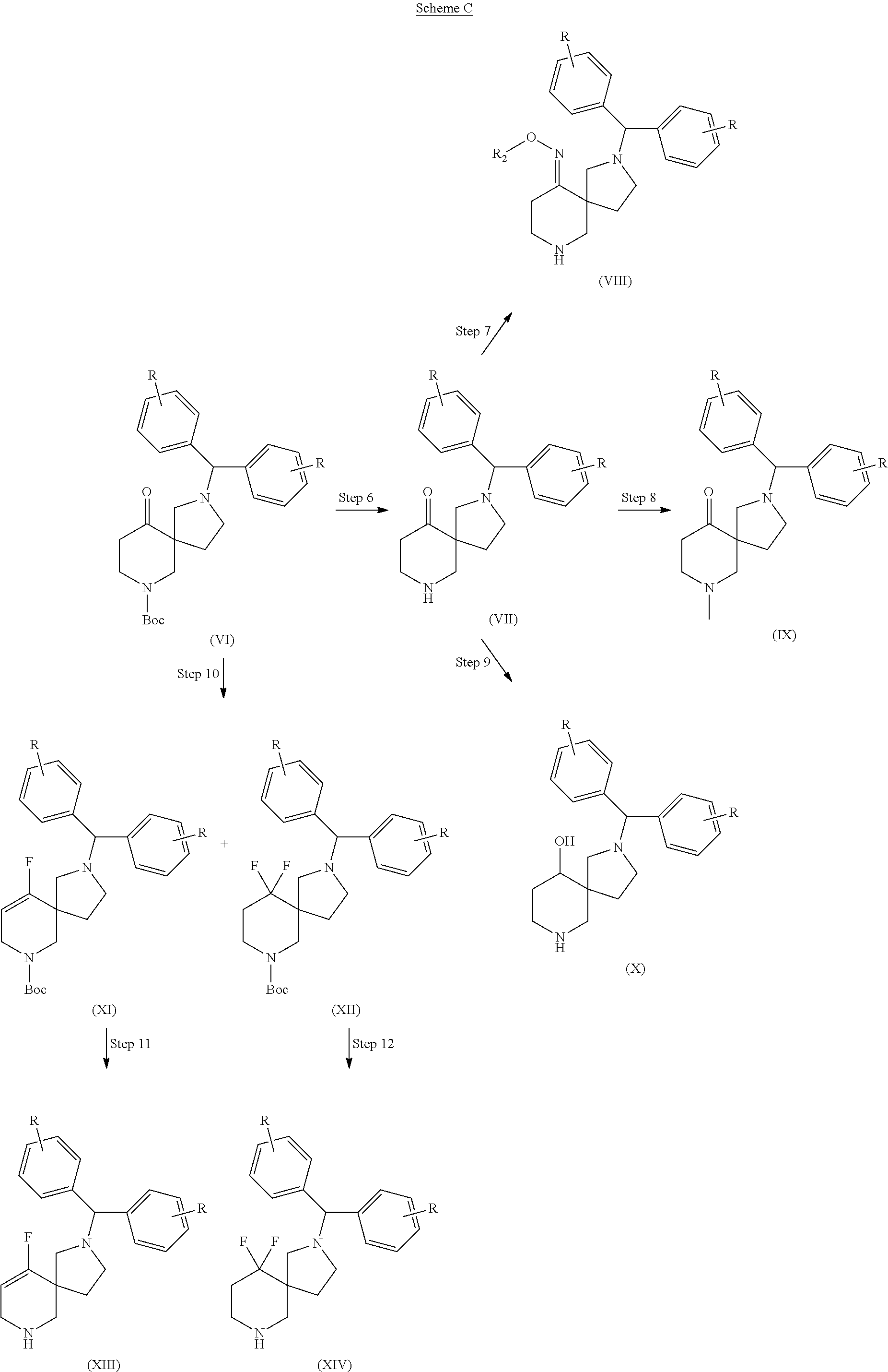

Step 6

Compound VII can be obtained from compound VI by removing the Boc group under acidic conditions, e. g. TFA in dichloromethane solution, typically at room temperature. The reaction takes about 1 hour.

Step 7

Compound of formula VII may be obtained by reductive amination of compound VI by reaction with an appropriate hydroxylamine, in a mixture of EtOH/water, in presence of a suitable base, such as aqueous NaOH. The reaction is carried out typically at reflux temperature and takes from about 1 hour to 12 hours to complete.

Step 8

Compound of formula IX may be obtained by reductive amination of compound VI by reaction with formaldehyde, in a suitable solvent, such as dichloromethane, in presence of a reducing agent like Na(AcO)₃BH. The reaction is carried out typically at room temperature and takes about 1 hour to complete.

Step 9

Compound of formula X can be obtained by reduction of compound VI with a suitable reducing agent, such as NaBH₄, in a suitable solvent, such as MeOH. The reaction is carried out typically at room temperature and takes about 1 hour to complete.

Step 10

Compounds of formula XI and XII can be obtained treating compound X with a fluorinating agent, such as DAST®, in a suitable solvent, e. g. dichloromethane. Typically the reaction proceeds at room temperature and takes 12 hours to complete.

Step 11

Compound XIII can be obtained from compound XI by removing the Boc group under acidic conditions, e. g. TFA in dichloromethane solution, typically at room temperature. The reaction takes about 0.5 hour.

Step 12

Compound XIV can be obtained from compound XII by removing the Boc group under acidic conditions, e. g. TFA in dichloromethane solution, typically at room temperature. The reaction takes about 0.5 hour.

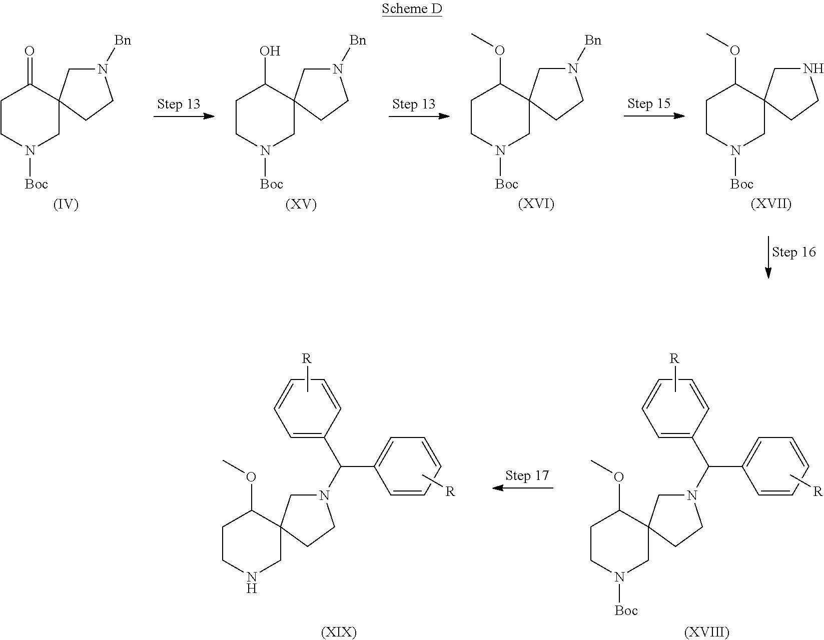

Step 13

Compound of formula XV can be obtained by reduction of compound IV with a suitable reducing agent, such as NaBH₄, in a suitable solvent, such as MeOH. The reaction is carried out typically at room temperature and takes about 1 hour to complete.

Step 14

Compound of formula XVI may be obtained by O-alkylation of compound XV by reaction with methyl iodide, after deprotonation with a suitable base, such as NaH, in aprotic solvent, such as DMF. The reaction is carried out typically at room temperature and takes about 16 hours to complete.

Step 15

Compound of formula XVII may be obtained by removing the benzyl group treating compound XVI with 1-chloroethyl chloroformate in a suitable solvent, such as dichloromethane, in presence of a suitable base, e. g. diisopropylamine, typically at reflux temperature for about 2 hours, followed by reflux in MeOH for about 1 hour.

Step 16

Compound XVIII can be obtained from compound XVII by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 5 hours to complete.

Step 17

Compound XIX can be obtained from compound XVIII by removing the Boc group under acidic conditions, e. g. TFA in dichloromethane solution, typically at room temperature. The reaction takes about 1 hour.

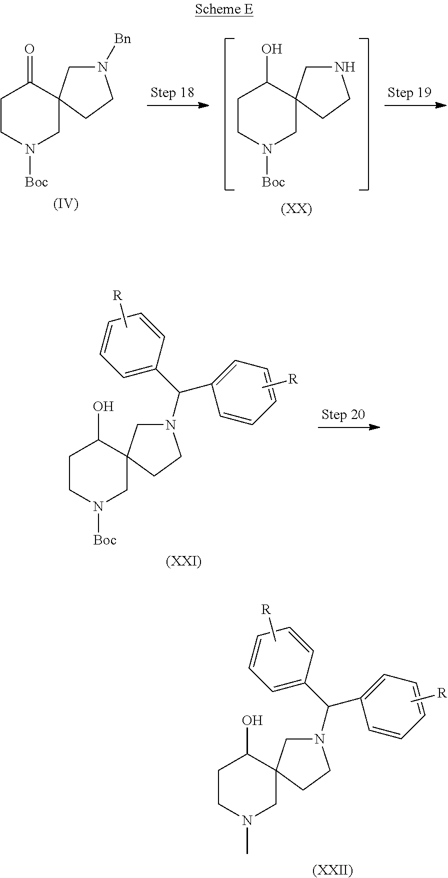

Step 18

Compound of formula XX may be obtained stirring compound XXI in hydrogen atmosphere, in presence of a suitable catalyst, such as palladium on carbon, in a suitable solvent such as methanol. The reaction proceeds at room temperature and takes about 1 hour.

Step 19

Compound XXI can be obtained from compound XX by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 12 hours to complete.

Step 20

Compound XXII can be obtained by treatment with LiAlH₄ of compound XXI, carrying out the reaction in a suitable solvent, e. g. THF, at reflux temperature. The reaction typically takes 1 hour to complete.

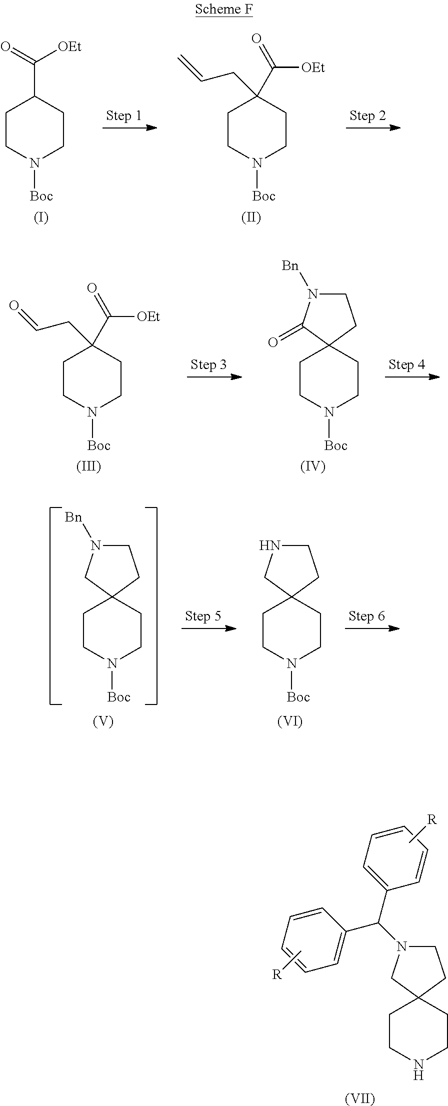

Step 1

Compound of formula II may be obtained by alkylation of compound I (commercially available from Sigma-Aldrich) with allyl bromide, after deprotonation using a suitable base, such as LiHMDS, in a suitable aprotic solvent, e. g. THF, carrying out the reaction at a temperature between −78° C. and room temperature. The reaction takes about 12 hours to complete.

Step 2

Compound of formula III may be obtained by oxidation of compound II using an aqueous solution of OsO₄, in a mixture of THF/water, in presence of NaIO₄, carrying out the reaction typically at room temperature. The reaction takes about 3 hours to complete.

Step 3

Compound of formula IV may be obtained by reductive amination and of compound Ill with a suitable primary amine, such as benzylamine, in a suitable solvent, such as THF, in presence of a reducing agent like Na(AcO)₃BH, followed by spontaneous lactam ring closure. The reaction is carried out typically at room temperature and takes about 12 hours to complete.

Step 4

Compound of formula V may be obtained by reduction of compound IV using a suitable reducing agent, e. g. LiAlH₄, carrying out the reaction in a suitable solvent, such as THF at a temperature between −20° C. to room temperature. The reaction takes about 2 hours to complete.

Step 5

Compound of formula VI may be obtained from compound V by removing the benzyl group by hydrogenolysis, e. g. using ammonium formate and palladium on carbon, in a suitable solvent such as methanol under reflux. The reaction takes about 1 hour.

Step 6

Compound VII may be obtained from compound VI by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 12 hours to complete. Then Boc removal was accomplished under acidic conditions, e. g. TFA in dichloromethane solution, typically at room temperature for about 1 hour.

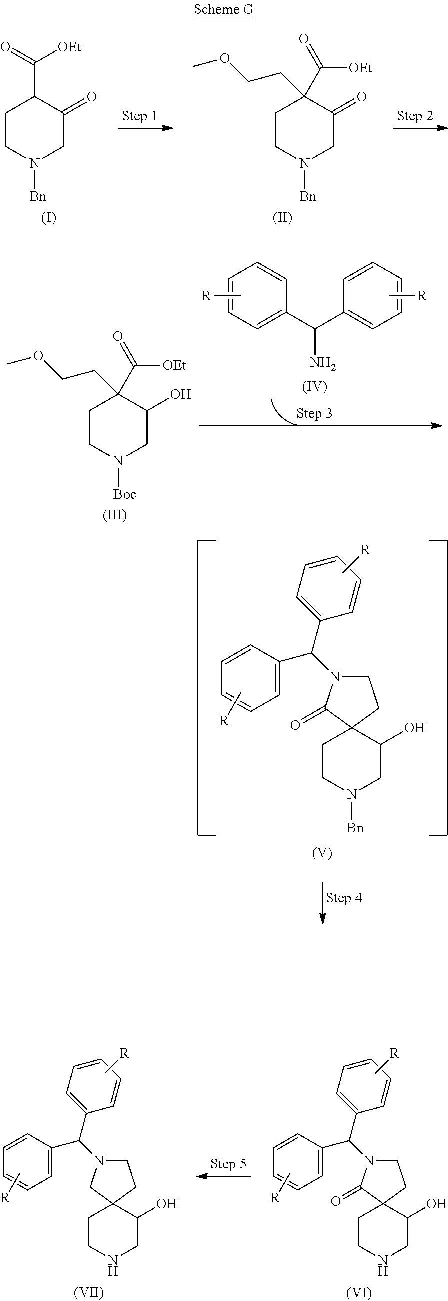

Step 1

Compound of formula II may be obtained by alkylation of compound I (commercially available from Sigma-Aldrich) with 1-Bromo-2-methoxyethane, after deprotonation using a suitable base, such as t-BuOK, in a suitable aprotic solvent, e. g. DMF, carrying out the reaction at a temperature of about 80° C. for 2 hours and then at 50° C. for 10 hours.

Step 2

Compound of formula III can be obtained by ketone reduction of compound II with a suitable reducing agent, such as NaBH₄, in a suitable solvent, such as MeOH. The reaction is carried out typically at room temperature and takes about 1 hour to complete.

Step 3

Compound of formula V may be obtained by lactam formation of compound III with compound IV (from scheme H), in a suitable solvent, such as toluene, in presence of a suitable Lewis acid, such as Et₂AlCl. The reaction is carried out typically at reflux temperature and takes about 24 hours to complete.

Step 4

Compound of formula VI can be obtained by benzyl of compound V by hydrogenolysis, e. g. under hydrogen atmosphere in presence of a suitable catalyst, such as palladium on carbon, in a suitable solvent such as methanol. The reaction takes about 16 hours.

Step 5

Compound of formula VII may be obtained by reduction of compound VI using a suitable reducing agent, e. g. LiAlH₄, carrying out the reaction in a suitable solvent, such as THF, at reflux temperature. The reaction takes about 5 hours to complete.

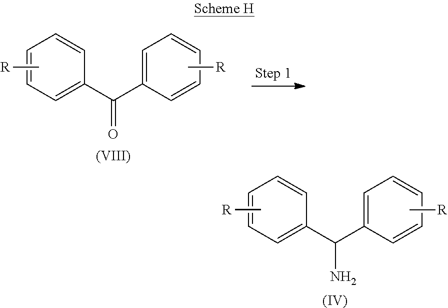

Step 1

Compound of formula IV can be obtained from a compound of formula VIII (for example, 4,4′-difluorobenzophenone, commercially available from Sigma-Aldrich) by reaction with formamide, usually at high temperature, such as 175° C., for about 18 hours and following treatment with aqueous solution of NaOH/ethanol at reflux temperature, typically for 2 hours.

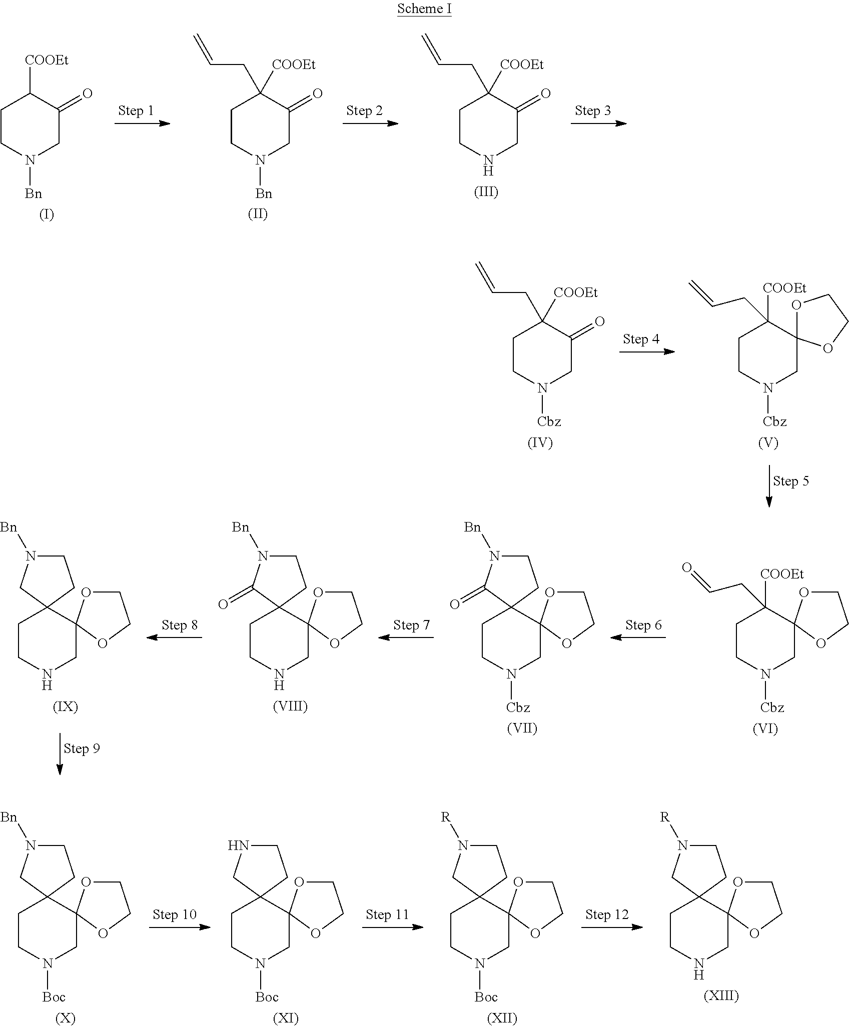

Step 1

Compound of formula II may be obtained by alkylation of compound I (commercially available from Sigma-Aldrich) with allyl bromide, after deprotonation using a suitable base, such as tBuOk, in a suitable aprotic solvent, e. g. THF, carrying out the reaction at a temperature between 0° C. and room temperature. The reaction takes about 12 hours to complete.

Step 2

Compound of formula III may be obtained by removing the benzyl group treating compound II with 1-chloroethyl chloroformate in a suitable solvent, such as dichloroethane, typically at reflux temperature for about 14 hours, followed by reflux in MeOH for about 1.5 hour.

Step 3

Compound of formula IV may be obtained by N-protection of compound III under standard literature conditions such as by reaction with a suitable protecting agent (e.g. as benzyl chloroformate), with the presence of a suitable base, such as diisopropylamine, carrying out the reaction in a suitable solvent, e.g. DCM, typically at room temperature. The reaction takes about 2 hours to complete.

Step 4

Compound of formula V may be obtained by ketone protection of compound IV by reaction with ethylene glycol, in presence of catalytic amount of p-Toluensulfonic in a suitable solvent, such as toluene, using Dean Stark apparatus, typically at reflux temperature. The reaction takes about 16 hours to complete.

Step 5

Compound of formula VI may be obtained by oxidation of compound V using an aqueous solution of OsO₄ in a mixture of THF/water, in presence of NaIO₄, carrying out the reaction typically at room temperature. The reaction takes about 2 hour to complete.

Step 6

Compound of formula VII may be obtained by reductive amination and of compound VI with a benzylamine, in a suitable solvent, such as THF, in presence of a reducing agent like Na(AcO)₃BH, followed by spontaneous lactam ring closure. The reaction is carried out typically at room temperature and takes about 16 to complete.

Step 7

Compound of formula VIII can be obtained by N-deprotection of compound VII by removing the benzyl group by hydrogenolysis, e. g. in hydrogen atmosphere with palladium on carbon, in a suitable solvent such as methanol. The reaction is carried out at a temperature about 25° C. The reaction takes from about 1.5 hours.

Step 8

Compound of formula IX may be obtained by reduction of compound VIII using a suitable reducing agent, e. g. LiAlH₄, carrying out the reaction in a suitable solvent, such as THF, at reflux temperature. The reaction takes to about 1.5 hour to complete.

Step 9

Compound of formula X may be obtained by N-protection of compound IX under standard literature conditions such as by reaction with Di-tert-butyl dicarbonate in a mixture of THF/water, in presence of a suitable base, such as Na₂CO₃, at a temperature around 0° C. The reaction takes about 1 hour to complete.

Step 10

Compound of formula XI may be obtained from compound X by removing the benzyl group by hydrogenolysis, e. g. using ammonium formate and palladium on carbon, in a suitable solvent such as methanol under reflux. The reaction takes about 2 hours.

Step 11

Compound XII may be obtained from compound XI by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 12 hours to complete.

Step 12

Compound XIII can be obtained from compound XII by removing the Boc group under acidic conditions, e. g. TFA in dichloromethane solution, typically at room temperature. The reaction takes about 12 hours.

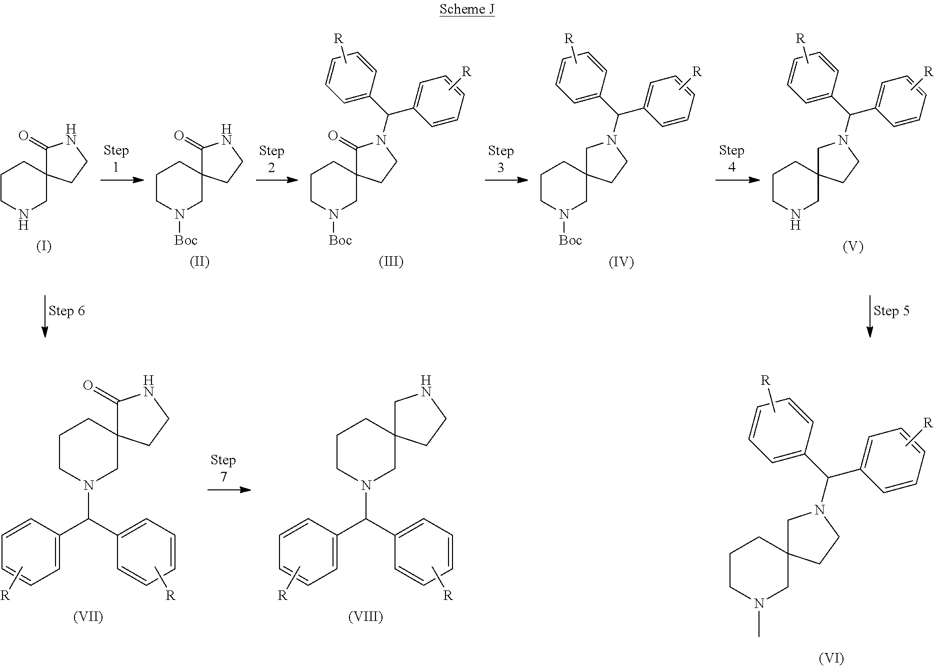

Step 1

Compound of formula II may be obtained by N-protection of compound I (commercially available from Bepharm Limited) under standard literature conditions such as by reaction with Di-tert-butyl dicarbonate, in presence of a suitable base such as triethylamine, carrying out the reaction in a suitable solvent, e.g. DCM, typically at room temperature. The reaction takes about 4 hours to complete.

Step 2

Compound III may be obtained from compound II by alkylation reaction using the appropriate benzhydryl chloride, such as Chlorobis(4-fluorophenyl)methane, after deprotonation with a suitable base, e. g. NaH, and carrying out the reaction in aprotic solvents, such as DMF. The reaction proceeds at a temperature of about 100° C. The reaction typically takes 12 hours to complete.

Step 3

Compound of formula IV may be obtained by reduction of compound III using a suitable reducing agent, e. g. BH₃ Me₂S complex, carrying out the reaction in a suitable solvent, such as THF, typically at room temperature and for 16 hours, followed by treatment with MeOH (at room temperature for about 20 hours).

Step 4

Compound of formula V can be obtained from compound of formula IV by removing the Boc group under acidic conditions, e. g. TFA in dichloromethane solution, typically at room temperature. The reaction takes about 1 hour.

Step 5

Compound of formula VI may be obtained by reductive amination of compound V by reaction with formaldehyde, in a suitable solvent, such as dichloromethane, in presence of a reducing agent like Na(AcO)₃BH. The reaction is carried out typically at room temperature and takes about 1 hour to complete.

Step 6

Compound VII may be obtained from compound I (commercially available from Bepharm Limited) by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 3 hours to complete.

Step 7

Compound of formula VIII may be obtained by reduction of compound VII using a suitable reducing agent, e. g. BH₃ Me₂S complex, carrying out the reaction in a suitable solvent, such as THF typically at room temperature and for 16 hours), followed by treatment with MeOH (at room temperature for about 16 hours).

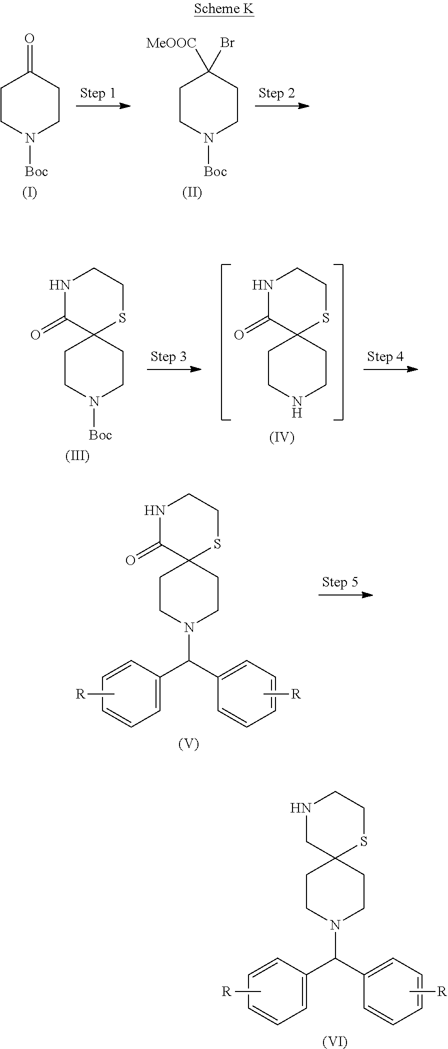

Step 1

Compound of formula II may be obtained from compound I (commercially available from Sigma-Aldrich) by reaction with bromoform, in a mixture of t-BuOH/water, in presence of a suitable base, such as LiOH H₂O and a phase transfer catalyst, e. g. benzyltriethylammonium chloride. The reaction is carried out at room temperature and takes about 72 hours to complete.

Step 2

Compound of formula III may be obtained by esterification of compound II, e. g. by reaction with Trimethylsilyl-diazomethane in a mixture toluene/methanol at room temperature for 3 hours, followed by cyclisation with 2-amino-ethanthiol in basic conditions, such as KOH in n-butanol. The reaction proceeds at reflux temperature and takes about 48 hours to complete.

Step 3

Compound IV can be obtained from compound III by removing the Boc group under acidic conditions, e. g. HCl in dichloromethane solution, typically at room temperature. The reaction takes about 12 hours to complete.

Step 4

Compound V may be obtained from compound IV by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 12 hours to complete

Step 5

Compound of formula VI may be obtained by reduction of compound V using a suitable reducing agent, e. g. LiAlH₄, carrying out the reaction in a suitable solvent, such as THF, at a temperature of about 60° C. The reaction takes to about 0.5 hour to complete.

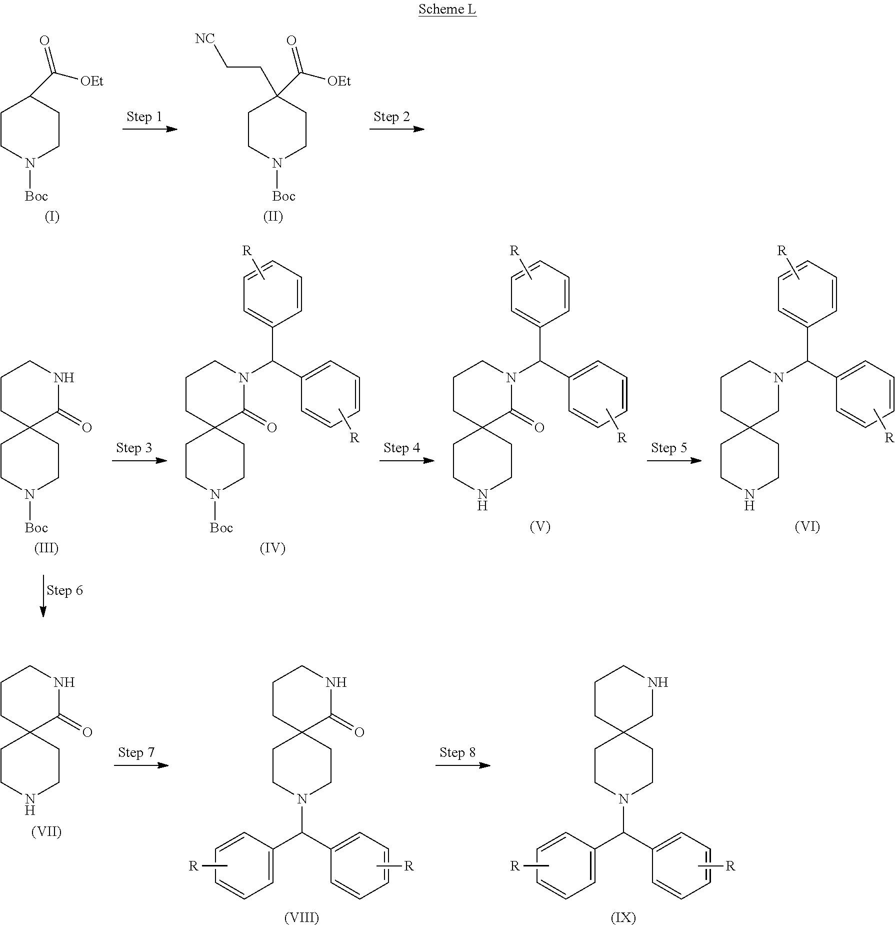

Step 1

Compound of formula II may be obtained by alkylation of compound I (commercially available from Sigma-Aldrich) with 3-bromopropanenitrile, after deprotonation using a suitable base, such as LDA, in a suitable solvent, e. g. THF, carrying out the reaction at a temperature between −78° C. and −30° C. The reaction takes about 4.5 hours to complete.

Step 2

Compound of formula III may be obtained by nitrile reduction and spontaneously lactam ring closure of compound II with a suitable reducing system, such as high pressure hydrogenation over PtO₂, in acid condition, such as a solution in CH₃COOH, typically for 12 hours at room temperature.

Step 3

Compound IV can be obtained by alkylation of compound III using the appropriate benzhydryl chloride, such as Chlorobis(4-fluorophenyl)methane, after deprotonation using a suitable base, such as NaH, in a suitable solvent, e. g. DMF, carrying out the reaction at a temperature of about 100° C. The reaction takes about 12 hours to complete.

Step 4

Compound V can be obtained from compound IV by removing the Boc group under acidic conditions, e. g. TFA in dichloromethane solution, typically at room temperature. The reaction takes about 2 hours.

Step 5

Compound of formula VI may be obtained by reduction of compound V using a suitable reducing agent, e. g. LiAlH₄, carrying out the reaction in a suitable solvent, such as THF at reflux temperature. The reaction takes about 2 hours to complete.

Step 6

Compound VII can be obtained from compound III by removing the Boc group under acidic conditions, e. g. HCl in dioxane solution, typically at room temperature. The reaction takes about 6 hours.

Step 7

Compound VIII may be obtained from compound VII by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 0.5 hour to complete.

Step 8

Compound of formula IX may be obtained by reduction of compound VIII using a suitable reducing agent, e. g. LiAlH₄, carrying out the reaction in a suitable solvent, such as THF at reflux temperature. The reaction takes about 2 hours to complete.

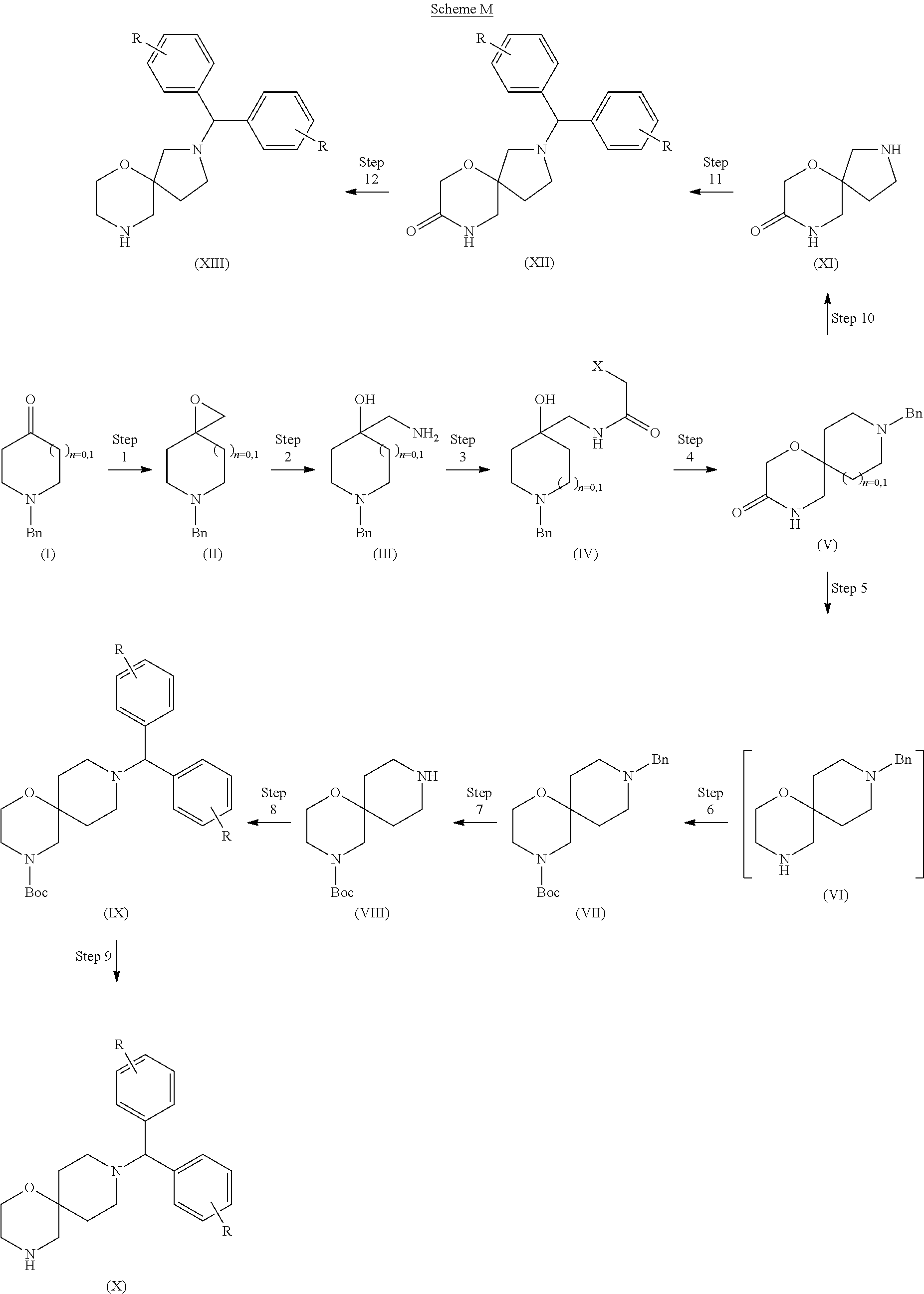

Step 1

Compound of formula II may be obtained by Corey-Chaykovsky epoxidation of compound I (commercially available from Sigma-Aldrich) using trimethylsulfoxonium iodide and an inorganic base, e. g. NaH, carrying out the reaction in a suitable solvent, such as DMSO, at room temperature. The reaction takes about 1 hour to complete.

Step 2

Compound of formula III may be obtained by epoxide opening of compound II using primary amines, such as ammonium hydroxide, carrying out the reaction in a mixture of MeOH/water, at room temperature. The reaction takes about 16 hours to complete.

Step 3

Compound IV may be obtained by acylation of compound III by reaction with an appropriate acylating agent (e. g. chloroacetyl chloride), with a suitable base, such as triethylamine, in a suitable solvent, such as dichloromethane, at a temperature between 0° C. and room temperature. The reaction takes from 30 minutes to 4 hours to complete.

Step 4

Compound of formula V can be obtained by ring closure of compound IV in an aprotic solvent, such as THF, in presence of a suitable base, e. g. NaH, at a temperature between 0° C. and room temperature.

The reaction takes from about 1 hour to about 2 hours to complete.

Step 5

Compound of formula VI may be obtained by reduction of compound V using a suitable reducing agent, e. g. LiAlH₄, carrying out the reaction in a suitable solvent, such as THF, and at elevate temperature (preferably at reflux). The reaction takes about 40 minutes to complete.

Step 6

Compound of formula VII may be obtained by N-protection of compound VI under standard literature conditions such as by reaction with Di-tert-butyl dicarbonate in a mixture of THF/water, in presence of a suitable base, such as Na₂CO₃, at a temperature of about 0° C. The reaction takes about 1 hour to complete.

Step 7

Compound of formula VIII may be obtained from compound VII by removing the benzyl group by hydrogenolysis, e. g. using ammonium formate and palladium on carbon, in a suitable solvent such as methanol under reflux. The reaction takes about 1 hour.

Step 8

Compound IX can be obtained by alkylation of compound VIII by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 7 hours to complete.

Step 9

Compound X can be obtained from compound IX by removing the Boc group under acidic conditions, e. g. TFA in dichloromethane solution, typically at room temperature. The reaction takes about 1 hour.

Step 10

Compound of formula XI may be obtained from compound V by removing the benzyl group by hydrogenolysis, e. g. using ammonium formate and palladium on carbon, in a suitable solvent such as methanol under reflux. The reaction takes about 1 hour.

Step 11

Compound XII can be obtained by alkylation of compound XI by alkylation reaction using the appropriate benzhydryl chloride, such as Chlorobis(4-fluorophenyl)methane, in the presence of an inorganic base, e. g. K₂CO₃, and carrying out the reaction in aprotic solvents, e. g. acetonitrile, at reflux temperature. The reaction typically takes 1 hour to complete.

Step 12

Compound of formula XIII may be obtained by reduction of compound XII using a suitable reducing agent, e. g. LiAlH₄, carrying out the reaction in a suitable solvent, such as THF, and at elevate temperature (preferably at reflux). The reaction takes about 1 hour to complete.

### EXAMPLES

The invention is further illustrated by the following non-limiting examples.

In the procedures that follow, after each starting material, reference to a Preparation or Example by number is typically provided. This is provided merely for assistance to the skilled chemist. The starting material may not necessarily have been prepared from the batch referred to.

Where reference is made to the use of a “similar or analogous” procedure, as will be appreciated by those skilled in the art, such procedure may involve minor variation, for example reaction temperature, reagent/solvent amount, reaction time, work-up conditions or chromatographic purification conditions. All temperatures refer to ° C.

Proton Magnetic Resonance (NMR) spectra may be typically recorded either on Varian instruments at 400 or 500 MHz, or on a Bruker instrument at 400 MHz.

Chemical shifts are expressed in parts of million (ppm, δ units). Chemical shifts are reported in ppm downfield (δ) from Me₄Si, used as internal standard, and are typically assigned as singlets (s), broad singlets (br.s.), doublets (d), doublets of doublets (dd), doublets of doublets of doublets (ddd), doublets of triplets (dt), triplets (t), triplets of doublets (td), quartets (q), or multiplets (m).

LCMS may be recorded under the following conditions:

DAD chromatographic traces, mass chromatograms and mass spectra may be taken on UPLC/PDA/MS Acquity™ system coupled with Micromass ZQ™ or Waters SQD single quadrupole mass spectrometer operated in positive and/or negative ES ionisation mode. The QC methods used were two, one operated under low pH conditions and another one operated under high pH conditions. Details of the method operated under low pH conditions were: column, Acquity BEH C₁₈, 1.7 μm, 2.1×50 mm or Acquity CSH C₁₈, 1.7 μm, 2.1×50 mm, the temperature column was 40° C.; mobile phase solvent A was milliQ water+0.1% HCOOH, mobile phase solvent B MeCN+0.1% HCOOH. The flow rate was 1 ml/min. The gradient table was t=0 min 97% A-3% B, t=1.5 min 0.1% A-99.9% B, t=1.9 min 0.1% A-99.9% B and t=2 min 97% A-3% B. The UV detection range was 210-350 nm and the ES⁺/ES⁻ range was 100-1000 amu.

Details of the method operated under high pH conditions were the same of those listed above for the low pH method apart from: column Acquity BEH C₁₈, 1.7 am, 2.1×50 mm; mobile phase solvent A was 10 mM aqueous solution of NH₄HCO₃ adjusted to pH=10 with ammonia, mobile phase solvent B MeCN. Semipreparative mass directed autopurifications (MDAP) were carried out using Waters Fractionlynx™ systems operated under low or high pH chromatographic conditions. The stationary phases used were, XTerra C18, XBridge C18, Sunfire C18, XSelect C18, Gemini AXIA C18. The length of the columns was 5, or 15 cm, while the internal diameter was 19, 21 or 30 mm. The particle size of the stationary phases was 5 or 10 am. The purifications were carried out using low pH or high pH chromatographic conditions. The mobile phase solvent composition was the same used for QC analysis. The combinations stationary/mobile phases used were: XTerra, XBridge, Sunfire, XSelect—low pH mobile phases and XTerra, XBridge, Gemini AXIA—high pH mobile phases. All the purifications were carried out with the column kept at room T. The flow rate used was 17 or 20 ml/min for columns of internal diameter 19 or 21 mm and 40 or 43 ml/min for columns of internal diameter 30 mm. The trigger for the collection of the target species was the presence of the target m/z ratio value in the TIC MS signal. The gradient timetable was customised on the Rt behaviour of the target species.

Purification may also be performed using Biotage® Isolera or Biotage® SP1 flash chromatography systems, these instruments work with Biotage® KP-SIL cartridges and Biotage® KP-NH cartridges.

Unless otherwise stated, all reactions are typically performed under inert atmosphere (for example under Nitrogen).

The following abbreviations are used in the text: EtOAc, AcOEt, EA=ethyl acetate, Et₂O=diethyl ether, MeOH=methanol; THF=tetrahydrofuran, dried refers to a solution dried over anhydrous sodium sulphate, r.t. (RT) refers to room temperature, DMSO=dimethyl sulfoxide; DMF=N,N′-dimethylformamide, DCM=dichloromethane, EtOH=ethanol, DCE=dichloroethane, Cy, cHex=cyclohexane, TEA=triethylamine, DIPEA=N,N-Diisopropylethylamine, Boc₂O=Di-tert-butyl dicarbonate; TFA=trifluoroacetic acid, ACE-Cl=1-chloroethyl chloroformate, LDA=lithium diisopropylamide, LiHMDS=lithium bis(trimethylsilyl)amide, SCX Cartridge=Strong Cation Exchange Cartridge.

### Preparation 1: tert-butyl 5-(2-ethoxy-2-oxoacetyl)-4-hydroxy-1,2,3,6-tetrahydropyridine-1-carboxylate (P1)

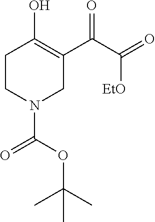 [SMILES: CCOC(=O)C(=O)C1=C(O)CCN(C(=O)OC(C)(C)C)C1]

A solution of LiOEt 1M in EtOH (22 mL) was cooled to 0° C., and diethyl oxalate (2.987 mL, 22 mmol) was added. To this mixture 1-Boc-4-piperidinone (5 g, 20 mmol) was added. The reaction mixture was stirred for 12 hrs, after which EtOH was removed under vacuum. The residue was diluted with Et₂O (500 mL), cooled to 0° C., and 1N HCl was added slowly until pH 5. Phases were separated and the organic layer was washed with H₂O (4×100 mL) and brine (100 mL). The organic layer was dried, filtered and concentrated. The residue was purified by FC on silica gel (eluent: cHex to cHex/EtOAc 70/30) affording tert-butyl 5-(2-ethoxy-2-oxoacetyl)-4-hydroxy-1,2,3,6-tetrahydropyridine-1-carboxylate (p1, 1.65 g, y=27%) as an orange oil.

MS (ES) (m/z): 300.02 [M+H]⁺.

### Preparation 2: tert-butyl 5-(2-ethoxy-2-oxoacetyl)-4-hydroxy-1,2,3,6-tetrahydropyridine-1-carboxylate (P2)

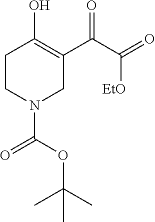 [SMILES: CCOC(=O)C(=O)C1=C(O)CCN(C(=O)OC(C)(C)C)C1]

A solution of LiHMDS 1M in THF (25 mL) was added to 25 mL of Et₂O and it was cooled to −78° C., 1-Boc-4-piperidone (5 g, 25 mmol) in Et₂O (25 mL) was added dropwise. The resulting yellow solution was stirred for 30 min, and then diethyl oxalate (3.4 mL, 25 mmol) in Et₂O (10 mL) was added. The reaction mixture was allowed to reach RT and stirred for 12 hrs.

The solution was cooled to 0° C., and 1N HCl was added slowly until pH 5. Phases were separated and the organic layer was washed with H₂O and brine. The organic layer was dried, filtered and concentrated. The crude material was purified by FC on silica gel (eluent: Cy to Cy/EA 70/30) to give tert-butyl 5-(2-ethoxy-2-oxoacetyl)-4-hydroxy-1,2,3,6-tetrahydropyridine-1-carboxylate (p2, 5.50 g, y=74%) as a yellow oil.

MS (ES) (m/z): 300.02 [M+H]⁺.

### Preparation 3: tert-butyl 3-methylidene-4-oxopiperidine-1-carboxylate (P3)

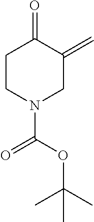

To a mixture of tert-butyl 5-(2-ethoxy-2-oxoacetyl)-4-hydroxy-1,2,3,6-tetrahydropyridine-1-carboxylate (p2, 1.65 g, 7.2 mmol) and aqueous formaldehyde (37%, 1.35 mL, 21.6 mmol) in THF (15 mL), a solution of NaOH (0.22 g, 7.2 mmol) and H₂O (0.8 mL) was added slowly. The reaction mixture was stirred for 20 min. The reaction mixture was then diluted with Et₂O (30 mL) and washed with H₂O (2×15 mL) and brine (50 mL). The organic layer was dried, filtered and concentrated (water bath temperature<25° C.) to afford tert-butyl 3-methylidene-4-oxopiperidine-1-carboxylate (p3, 1.5 g, y=quant.) as colorless oil, which was immediately used in the next reaction without further purification.

¹H NMR (400 MHz, CHLOROFORM-d): δ ppm 6.13 (s, 1H) 5.39 (br. s., 1H) 4.37 (br. s., 2H) 3.71-3.81 (m, 2H) 2.59 (t, J=6.34 Hz, 2H) 1.44-1.55 (s, 9H)

### Preparation 4: tert-butyl 2-benzyl-10-oxo-2,7-diazaspiro[4.5]decane-7-carboxylate (P4)

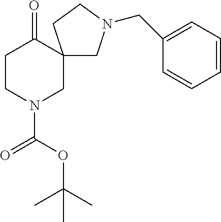 [SMILES: CC(C)(C)OC(=O)N1CCC(=O)C2(CCN(Cc3ccccc3)C2)C1]

To a stirred solution of tert-butyl 3-methylidene-4-oxopiperidine-1-carboxylate (p3, 1.75 g, 8.28 mmol) in DCM (20 mL), at 0° C. under Argon, TFA (0.19 mL) was added followed by N-(Methoxymethyl)-N-(trimethylsilylmethyl)benzylamine (1.70 mL, 6.63 mmol) dropwise, keeping the reaction temperature below 5° C. After 5 min the ice-bath was removed and the reaction was stirred at RT for 12 hrs. The organic layer was extracted, washed with Na₂CO₃ and brine. The organic layer was dried, filtered and concentrated. The crude material was purified by FC on silica gel (eluent Cy to Cy/EA 70/30) to give tert-butyl 2-benzyl-10-oxo-2,7-diazaspiro[4.5]decane-7-carboxylate (p4, 545 mg, y=20%) as a colourless oil.

MS (ES) (m/z): 345.2 [M+H]⁺.

### Preparation 5: tert-butyl 10-oxo-2,7-diazaspiro[4.5]decane-7-carboxylate (P5)

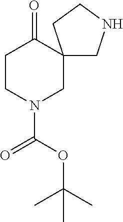 [SMILES: CC(C)(C)OC(=O)N1CCC(=O)C2(CCNC2)C1]

ACE-Cl (0.094 mL, 0.871 mmol) was added to a solution of tert-butyl 2-benzyl-10-oxo-2,7-diazaspiro[4.5]decane-7-carboxylate (p4, 0.3 g, 0.871 mmol) and DIPEA (0.152 mL, 0.871 mmol) in 4 mL of DCM. The solution was stirred at reflux (45° C.) for 2 hrs, then it was dried, redissolved with MeOH (3 mL) and refluxed (70° C.) for 1 h. The solvent was evaporated, the residue was dissolved with DCM and washed with H₂O. The organic phase was dried and evaporated to obtain tert-butyl 10-oxo-2,7-diazaspiro[4.5]decane-7-carboxylate (p5, 180 mg, y=81%) as yellow oil which was used in the next step without further purification.

MS (ES) (m/z): 373.2 [M+H]⁺.

### Preparation 6: tert-butyl 2-[bis(4-fluorophenyl)methyl]-10-oxo-2,7-diazaspiro[4.5]decane-7-carboxylate (P6)

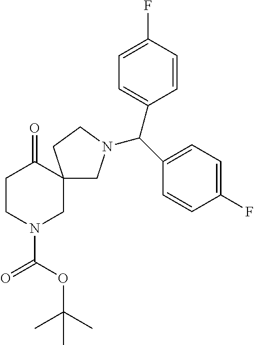 [SMILES: CC(C)(C)OC(=O)N1CCC(=O)C2(CCN(C(c3ccc(F)cc3)c3ccc(F)cc3)C2)C1]

Chlorobis(4-fluorophenyl)methane (0.158 mL, 0.849 mmol) was added to a stirred mixture of tert-butyl 10-oxo-2,7-diazaspiro[4.5]decane-7-carboxylate (p5, 180 mg, 0.708 mmol) and K₂CO₃ (245 mg, 1.77 mmol) in Acetonitrile (5 mL). The mixture was stirred for 3 hrs at reflux. The solution was filtered using EtOAc and evaporated to obtain tert-butyl 2-[bis(4-fluorophenyl)methyl]-10-oxo-2,7-diazaspiro[4.5]decane-7-carboxylate (p6, 350 mg, crude material) as orange oil which was used in the next step without further purification.

MS (ES) (m/z): 457.3 [M+H]⁺.

### Example 1: 2-[bis(4-fluorophenyl)methyl]-2,7-diazaspiro[4.5]decan-10-one (E1)

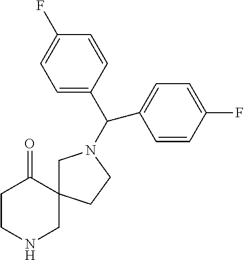 [SMILES: O=C1CCNCC12CCN(C(c1ccc(F)cc1)c1ccc(F)cc1)C2]

TFA (1 mL) was added to a solution of tert-butyl 2-[bis(4-fluorophenyl)methyl]-10-oxo-2,7-diazaspiro[4.5]decane-7-carboxylate (p6, 350 mg, 0.767 mmol) in 5 mL of DCM. The mixture was stirred for 1 h, and then the solvent was removed under reduced pressure. The residue was charged on SCX cartridge washing with MeOH and eluting with 1M NH₃ in MeOH to afford 2-[bis(4-fluorophenyl)methyl]-2,7-diazaspiro[4.5]decan-10-one as colorless oil (E1, 20 mg, y=7%).

MS (ES) (m/z): 357.2 [M+H]⁺.

### Example 2: 2-[bis(4-fluorophenyl)methyl]-7-methyl-2,7-diazaspiro[4.5]decan-10-one (E2)

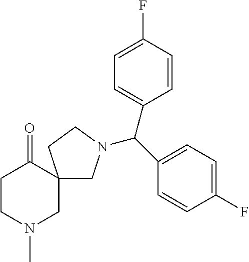 [SMILES: CN1CCC(=O)C2(CCN(C(c3ccc(F)cc3)c3ccc(F)cc3)C2)C1]

To a solution of 2-[bis(4-fluorophenyl)methyl]-2,7-diazaspiro[4.5]decan-10-one (E1, 20 mg, 0.056 mmol) in DCM (1.5 mL) formaldehyde 37% w/w in water (0.041 mL, 0.56 mmol) was added and the mixture was stirred at RT for 15 min. NaBH(OAc)₃ (17 mg, 0.084 mmol) was then added and the mixture was stirred at RT for 1 h. The reaction was concentrated under reduced pressure and loaded on SCX cartridge washing with MeOH and eluting with 1M NH₃ in MeOH.

After evaporation of combined ammonia fractions the residue was purified by FC on NH column (eluent: cHex to cHex/EtOAc 60/40) to afford 2-[bis(4-fluorophenyl)methyl]-7-methyl-2,7-diazaspiro[4.5]decan-10-one (E2, 13.6 mg, y=65%) as white foam.

MS (ES) (m/z): 371.2 [M+H]⁺.

¹H NMR (CHLOROFORM-d): δ ppm 7.38 (dd, 4H) 6.90-7.03 (m, 4H) 4.18 (s, 1H) 2.44-2.73 (m, 9H) 2.23-2.40 (m, 5H) 1.61-1.70 (m, 1H)

### Example 3: 2-[bis(4-fluorophenyl)methyl]-2,7-diazaspiro[4.5]decan-10-ol (E3)

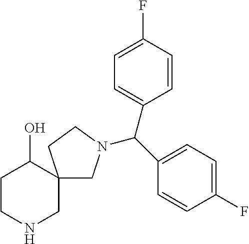 [SMILES: OC1CCNCC12CCN(C(c1ccc(F)cc1)c1ccc(F)cc1)C2]

To a stirred solution of 2-[bis(4-fluorophenyl)methyl]-2,7-diazaspiro[4.5]decan-10-one (E1, 16.3 mg, 0.046 mmol) in MeOH (0.5 mL), NaBH₄ (3.46 mg, 0.091 mmol). The reaction was stirred at RT for 1 h, and then methanol was removed. The residue was charged on SCX cartridge washing with MeOH and eluting with 1M NH₃ in MeOH to afford 2-[bis(4-fluorophenyl)methyl]-2,7-diazaspiro[4.5]decan-10-ol (E3, 17 mg, y=quant.).

MS (ES) (m/z): 359.2 [M+H]⁺.

¹H NMR (CHLOROFORM-d): δ ppm 7.30-7.40 (m, 4H), 6.93-7.05 (m, 4H), 4.07-4.15 (m, 1H), 3.37-3.65 (m, 1H), 2.77-3.21 (m, 2H), 2.42-2.75 (m, 2H), 1.36-2.26 (m, 8H)

### Preparation 7: tert-butyl 2-[bis(4-fluorophenyl)methyl]-10-hydroxy-2,7-diazaspiro[4.5]decane-7-carboxylate (P7)

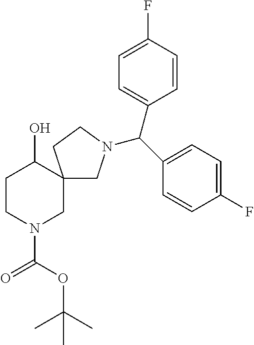 [SMILES: CC(C)(C)OC(=O)N1CCC(O)C2(CCN(C(c3ccc(F)cc3)c3ccc(F)cc3)C2)C1]

Step a:

To a solution of tert-butyl 2-benzyl-10-oxo-2,7-diazaspiro[4.5]decane-7-carboxylate (p4, 480 mg, 1.39 mmol) in MeOH (20 mL) 10% Pd/C (0.196 mg) was added and the mixture was stirred in a H₂ atmosphere at RT for 5 hrs.

The solution was filtered and evaporated to obtain tert-butyl 10-hydroxy-2,7-diazaspiro[4.5]decane-7-carboxylate (0.38 g) as white foam.

Step b:

tert-butyl 10-hydroxy-2,7-diazaspiro[4.5]decane-7-carboxylate (from step a, 0.38 g, 1.39 mmol) was dissolved in Acetonitrile (10 mL), K₂CO₃ (0.48 g, 4.17 mmol), NaI (0.25 g, 1.67 mmol) and Chlorobis(4-fluorophenyl)methane (0.311 mL, 1.67 mmol) were added. The mixture was heated at reflux for 12 hrs. The mixture was diluted with water and extracted with EtOAc (15 mL×3). The organic phase was dried and evaporated. The crude material was purified by FC on silica gel (eluent: cHex to cHex/EtOAc 50/50) affording tert-butyl 2-[bis(4-fluorophenyl)methyl]-10-hydroxy-2,7-diazaspiro[4.5]decane-7-carboxylate (p7, 120 mg, y=19%).

MS (ES) (m/z): 459.2 [M+H]⁺.

### Example 4: 2-[bis(4-fluorophenyl)methyl]-7-methyl-2,7-diazaspiro[4.5]decan-10-ol (E4)

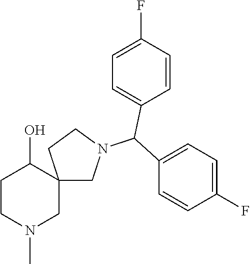 [SMILES: CN1CCC(O)C2(CCN(C(c3ccc(F)cc3)c3ccc(F)cc3)C2)C1]

tert-butyl 2-[bis(4-fluorophenyl)methyl]-10-hydroxy-2,7-diazaspiro[4.5]decane-7-carboxylate (p7, 120 mg, 0.26 mmol) was dissolved in THF (4 mL), then LiAlH₄ 2M in THF (0.35 mL, 0.68 mmol) was added dropwise. The solution was heated to reflux and stirred for 1 h. Then it was cooled down to RT and Na₂SO₄ 10 H₂O was added, followed by MgSO₄. The mixture was stirred for 30 min, and then filtered washing with EtOAc.

The solvent was removed under vacuum, and the crude material was purified by FC on silica gel (eluent: DCM to DCM/MeOH 90:10) to obtain 2-[bis(4-fluorophenyl)methyl]-7-methyl-2,7-diazaspiro[4.5]decan-10-ol (E4, 46 mg, y=47%) as white solid.

MS (ES) (m/z): 373.2 [M+H]⁺.

¹H NMR (CHLOROFORM-d): δ ppm 7.46 (ddd, 4H), 7.04-7.16 (m, 4H), 4.39 (d, 1H), 4.27 (s, 1H), 3.22-3.31 (m, 1H), 2.42-2.51 (m, 3H), 2.19-2.34 (m, 3H), 2.14 (s, 3H), 1.83-2.04 (m, 3H), 1.38-1.60 (m, 3H)

### Preparation 8: tert-butyl 2-benzyl-10-hydroxy-2,7-diazaspiro[4.5]decane-7-carboxylate (P8)

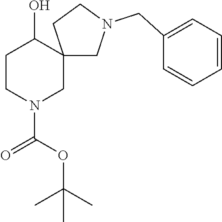 [SMILES: CC(C)(C)OC(=O)N1CCC(O)C2(CCN(Cc3ccccc3)C2)C1]

To a stirred solution of tert-butyl 2-benzyl-10-oxo-2,7-diazaspiro[4.5]decane-7-carboxylate (p4, 270 mg, 0.784 mmol) in MeOH (6 mL) NaBH₄ (59.31 mg, 1.57 mmol) was added. The reaction was stirred at RT for 1 h. Water was added, MeOH was removed and the product was extracted with DCM. The organic phase was dried and concentrated under reduced pressure. The residue was purified by FC on NH column (eluent: Cy to Cy/EtOAc 50/50) affording tert-butyl 2-benzyl-10-hydroxy-2,7-diazaspiro[4.5]decane-7-carboxylate (p8, 180 mg, y=66%).

MS (ES) (m/z): 347.27 [M+H]⁺.

### Preparation 9: tert-butyl 2-benzyl-10-methoxy-2,7-diazaspiro[4.5]decane-7-carboxylate (P9)

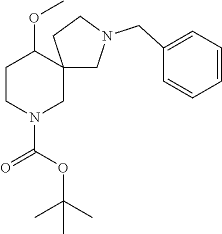 [SMILES: COC1CCN(C(=O)OC(C)(C)C)CC12CCN(Cc1ccccc1)C2]

NaH 60% dispersion in mineral oil (22.86 mg, 0.57 mmol) was added to a stirred solution of tert-butyl 2-benzyl-10-hydroxy-2,7-diazaspiro[4.5]decane-7-carboxylate (p8, 180 mg, 0.519 mmol) in DMF (3 mL) at 0° C. under Argon. After 30 min, MeI (0.036 mL, 0.57 mmol) were added and the reaction was stirred at RT for 16 hrs. The solvent was removed; the residue was diluted with DCM and washed with H₂O. The organic phase was dried and evaporated. The crude material was purified by FC on NH column (eluent: cHex to cHex/EtOAc 60/40) affording tert-butyl 2-benzyl-10-methoxy-2,7-diazaspiro[4.5]decane-7-carboxylate (p9, 49 mg, y=26%, purity 50%) as colourless oil.

MS (ES) (m/z): 361.25 [M+H]⁺.

### Preparation 10: tert-butyl 10-methoxy-2,7-diazaspiro[4.5]decane-7-carboxylate (P10)

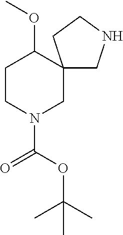 [SMILES: COC1CCN(C(=O)OC(C)(C)C)CC12CCNC2]

ACE-Cl (0.029 mL, 0.272 mmol) was added to a solution of tert-butyl 2-benzyl-10-methoxy-2,7-diazaspiro[4.5]decane-7-carboxylate (p9, 49 mg, 0.136 mmol) and DIPEA (0.024 mL, 0.136 mmol) in 2 mL of DCM. The solution was stirred at reflux (45° C.) for 2 hrs, then it was cooled down to RT, concentrated, redissolved with MeOH (1.5 mL) and refluxed (70° C.) for 1 h. The mixture was cooled down to RT and solvent was evaporated; the residue was dissolved with DCM and washed with H₂O. The organic phase was dried and concentrated under reduced pressure affording tert-butyl 10-methoxy-2,7-diazaspiro[4.5]decane-7-carboxylate (p10, 35 mg, crude material) as yellow oil which was used in the next step without further purification.

MS (ES) (m/z): 271.25 [M+H]⁺.

### Preparation 11: tert-butyl 2-[bis(4-fluorophenyl)methyl]-10-methoxy-2,7-diazaspiro[4.5]decane-7-carboxylate (P11)

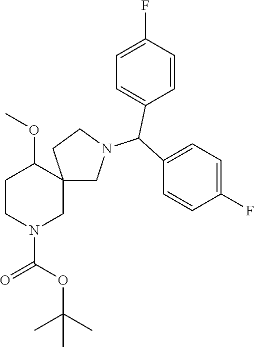 [SMILES: COC1CCN(C(=O)OC(C)(C)C)CC12CCN(C(c1ccc(F)cc1)c1ccc(F)cc1)C2]

Chlorobis(4-fluorophenyl)methane (0.029 mL, 0.155 mmol) was added to a stirred mixture of tert-butyl 10-methoxy-2,7-diazaspiro[4.5]decane-7-carboxylate (p10, 35 mg, 0.129 mmol) and K₂CO₃ (44.6 mg, 0.323 mmol) in Acetonitrile (1.5 mL). The mixture was stirred for 5 hrs at reflux. The solution was filtered washing with EtOAc and evaporated to obtain tert-butyl 2-[bis(4-fluorophenyl)methyl]-10-methoxy-2,7-diazaspiro[4.5]decane-7-carboxylate (p11, 65 mg, crude material) as orange oil.

MS (ES) (m/z): 473.28 [M+H]⁺.

### Example 5: 2-[bis(4-fluorophenyl)methyl]-10-methoxy-2,7-diazaspiro[4.5]decane (E5)

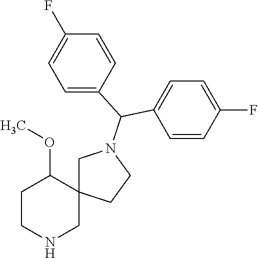 [SMILES: COC1CCNCC12CCN(C(c1ccc(F)cc1)c1ccc(F)cc1)C2]

TFA (0.5 mL) was added to a solution of tert-butyl 2-[bis(4-fluorophenyl)methyl]-10-methoxy-2,7-diazaspiro[4.5]decane-7-carboxylate (p11, 65 mg, 0.138 mmol) in 3 mL of DCM. The mixture was stirred at RT for 1 h, and then solvent was removed under reduced pressure. The crude material was purified by FC on NH column (eluent: cHex to cHex/EtOAc 50/50) affording 2-[bis(4-fluorophenyl)methyl]-10-methoxy-2,7-diazaspiro[4.5]decane (E5, 41 mg, y=80% as diastereomeric mixture 60/40) as colourless oil.

MS (ES) (m/z): 373.21 [M+H]⁺.

¹H NMR (CHLOROFORM-d): δ ppm 7.34-7.46 (m, 4H), 6.91-7.03 (m, 4H), 4.16 (s, 1H), 3.31-3.39 (d, 3H), 2.89-3.05 (m, 3H), 2.49-2.65 (m, 3H), 2.26-2.41 (m, 3H), 1.96 (m, 1H), 1.86-1.45-1.54 (m, 3H)

### Preparation 12: 1-benzyl 3-methyl 4-oxopiperidine-1,3-dicarboxylate (P12)

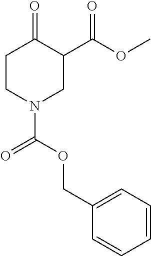 [SMILES: COC(=O)C1CN(C(=O)OCc2ccccc2)CCC1=O]

TEA (17.67 mL, 126.75 mmol) was added to a stirred solution of Methyl 4-oxo-3-piperidinecarboxylate hydrochloride (8.18 g, 42.25 mmol) in DCM (80 mL); the solution was cooled to 0° C. then benzyl chloroformate (6.93 mL, 48.58 mmol) was added dropwise. The resulting mixture was stirred at RT overnight. The mixture was washed with 1N HCl aq then with aq. NaHCO₃ saturated solution, organic phase was dried and concentrated under vacuum to give 1-benzyl 3-methyl 4-oxopiperidine-1,3-dicarboxylate (p12, 5.30 g, y=43%) as an orange oil.

MS (ES) (m/z): 292.2 [M+H]⁺.

### Preparation 13: 1-benzyl 3-methyl 4-oxo-3-(prop-2-en-1-yl)piperidine-1,3-dicarboxylate (P13)

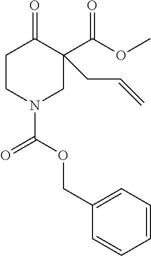 [SMILES: C=CCC1(C(=O)OC)CN(C(=O)OCc2ccccc2)CCC1=O]

To a solution of 1-benzyl 3-methyl 4-oxopiperidine-1,3-dicarboxylate (p12, 5.30 g, 18.19 mmol) in DMF (27 mL) NaH 60% dispersion in mineral oil (0.873 g, 21.83 mmol) was added at 0° C. After vigorous stirring for 1 h at RT, allyl bromide (1.62 mL, 18.74 mmol) was added and the mixture was stirred for 4 hrs. The reaction was quenched by addition of H₂O (25 mL) and extracted with EtOAc (3×25 mL). The organic phase was dried and concentrated under reduced pressure. The crude was purified by FC on silica gel (eluent: cHex to cHex/EtOAc 70/30) affording 1-benzyl 3-methyl 4-oxo-3-(prop-2-en-1-yl)piperidine-1,3-dicarboxylate (p13, 4.7 g, y=78%).

MS (ES) (m/z): 332.2 [M+H]⁺.

### Preparation 14: 8-benzyl 6-methyl 6-(prop-2-en-1-yl)-1,4-dioxa-8-azaspiro[4.5]decane-6,8-dicarboxylate (P14)

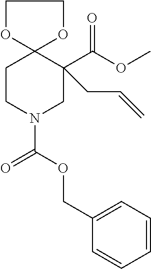 [SMILES: C=CCC1(C(=O)OC)CN(C(=O)OCc2ccccc2)CCC12OCCO2]

A mixture of 1-benzyl 3-methyl 4-oxo-3-(prop-2-en-1-yl)piperidine-1,3-dicarboxylate (p13, 4.70 g, 14.19 mmol), ethylene glycol (7.91 mL, 141.90 mmol) and p-Toluenesulphonic acid monohydrate (405 mg, 2.13 mmol) in dry toluene (25 mL) was heated under reflux for 16 hrs using a Dean-Stark apparatus. The mixture was cooled down to RT and concentrated under vacuum. The residue was dissolved in Et₂O and washed with water. Phases were separated and organic phase was dried and concentrated under reduced pressure affording 8-benzyl 6-methyl 6-(prop-2-en-1-yl)-1,4-dioxa-8-azaspiro[4.5]decane-6,8-dicarboxylate (p14, 4.90 g, y=92%) as colorless oil.

MS (ES) (m/z): 376.2 [M+H]⁺.

### Preparation 15: 8-benzyl 6-methyl 6-(2-oxoethyl)-1,4-dioxa-8-azaspiro[4.5]decane-6,8-dicarboxylate (P15)

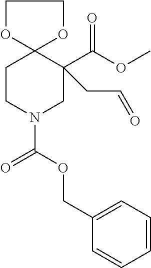 [SMILES: COC(=O)C1(CC=O)CN(C(=O)OCc2ccccc2)CCC12OCCO2]

8-benzyl 6-methyl 6-(prop-2-en-1-yl)-1,4-dioxa-8-azaspiro[4.5]decane-6,8-dicarboxylate (p14, 4.90 g, 13.05 mmol) was dissolved in THF/H₂O (25+25 mL). To this stirred mixture a solution of OsO₄ 4% in water (3 mL, 0.392 mmol) was added over 30 seconds and the resulting mixture was stirred at RT for 5 min. NaIO₄ (6.98 g, 32.63 mmol) was added and the mixture stirred for 1 h. The mixture was partitioned between NaHCO₃ (30 mL) and Et₂O (3×50 mL). The organic phase was dried and concentrated. The crude was purified by FC on silica gel (eluent: cHex to cHex/EtOAc 60/40) affording 8-benzyl 6-methyl 6-(2-oxoethyl)-1,4-dioxa-8-azaspiro[4.5]decane-6,8-dicarboxylate (p15, 2.95 g, y=60%) as colorless oil.

MS (ES) (m/z): 378.2 [M+H]⁺.

### Preparation 16: benzyl 8-benzyl-7-oxo-1,4-dioxa-8,12-diazadispiro[4.0.4⁶.4⁵]tetradecane-12-carboxylate (P16)

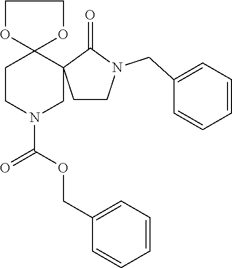 [SMILES: O=C(OCc1ccccc1)N1CCC2(OCCO2)C2(CCN(Cc3ccccc3)C2=O)C1]

To a solution of 8-benzyl 6-methyl 6-(2-oxoethyl)-1,4-dioxa-8-azaspiro[4.5]decane-6,8-dicarboxylate (p15, 2.95 g, 7.82 mmol) and benzylamine (1.11 mL, 10.16 mmol) in THF (30 mL), Na(AcO)₃BH (3.31 g, 15.64 mmol) was added. The resulting mixture was stirred at RT overnight. The mixture was partitioned between NaHCO₃ saturated solution and EtOAc. The organic phase was dried and concentrated. Crude material was purified by FC on silica gel (eluent: Cy to Cy/AcOEt 40/60) affording benzyl 8-benzyl-7-oxo-1,4-dioxa-8,12-diazadispiro[4.0.4⁶.4⁵]tetradecane-12-carboxylate (p16, 2.60 g, y=76%) as colourless oil.

MS (ES) (m/z): 437.3 [M+H]⁺.

### Preparation 17: 8-benzyl-1,4-dioxa-8,12-diazadispiro[4.0.4⁶.4⁵]tetradecan-7-one (P17)

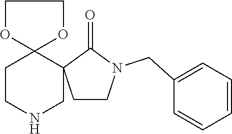 [SMILES: O=C1N(Cc2ccccc2)CCC12CNCCC21OCCO1]

Under a hydrogen atmosphere, a mixture of benzyl 8-benzyl-7-oxo-1,4-dioxa-8,12-diazadispiro[4.0.4⁶.4⁵]tetradecane-12-carboxylate (p16, 2.60 g, 5.96 mmol), 10% palladium on carbon (412 mg, 3.87 mmol), and MeOH (30 mL) was stirred at RT for 30 min. The Pd/C was filtered off, the mixture was washed with MeOH, and the filtrate was concentrated under reduced pressure affording 8-benzyl-1,4-dioxa-8,12-diazadispiro[4.0.4⁶.4⁵]tetradecan-7-one (p17, 1.80 g, y=quantitative) as colorless oil.

MS (ES) (m/z): 303.2 [M+H]⁺.

### Preparation 18: 8-benzyl-1,4-dioxa-8,12-diazadispiro[4.0.4⁶.4⁵]tetradecane (P18)

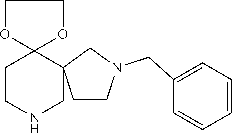 [SMILES: c1ccc(CN2CCC3(CNCCC34OCCO4)C2)cc1]

LiAlH₄ (4.45 mL, 8.93 mmol) was added to solution of 8-benzyl-1,4-dioxa-8,12-diazadispiro[4.0.4⁶.4⁵]tetradecan-7-one (p17, 1.80 g, 5.95 mmol) in THF (10 mL), then the mixture was heated to 65° C. and stirred at that temperature for 4 hrs. The reaction was cooled down to 0° C. and quenched with Na₂SO₄*10H₂O, the solid was filtered off, washed with EtOAc and the filtrate was concentrated under reduced pressure affording 8-benzyl-1,4-dioxa-8,12-diazadispiro[4.0.4⁶.4⁵]tetradecane (p18, 1.60 g, y=93%), which was used in the next step without further purification.

MS (ES) (m/z): 289.2 [M+H]⁺.

### Preparation 19: tert-butyl 8-benzyl-1,4-dioxa-8,12-diazadispiro[4.0.4⁶.4⁵]tetradecane-12-carboxylate (P19)

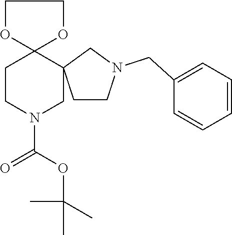 [SMILES: CC(C)(C)OC(=O)N1CCC2(OCCO2)C2(CCN(Cc3ccccc3)C2)C1]

8-benzyl-1,4-dioxa-8,12-diazadispiro[4.0.4⁶.4⁵]tetradecane (p18, 1.60 g, 5.54 mmol) was dissolved in H₂O (12 mL) at RT then cooled down to 0° C. Na₂CO₃ (0.572 g, 5.65 mmol) was added followed by the drop-wise addition of a solution of Boc₂O (1.20 g, 5.54 mmol) in THF (10 mL). The mixture was stirred at the same temperature for 1 h, and then worked up extracting with EtOAc. The organic phase was washed with brine, dried and concentrated under reduced pressure affording tert-butyl 8-benzyl-1,4-dioxa-8,12-diazadispiro[4.0.4⁶.4⁵]tetradecane-12-carboxylate (p19, 2.25 g, y=quant.) as colourless oil, which was used in the next step without further purification.

MS (ES) (m/z): 389.3 [M+H]⁺.

### Preparation 20: tert-butyl 1,4-dioxa-8,12-diazadispiro[4.0.4⁶.4⁵]tetradecane-12-carboxylate (P20)

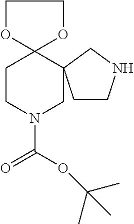 [SMILES: CC(C)(C)OC(=O)N1CCC2(OCCO2)C2(CCNC2)C1]

To a solution of tert-butyl 8-benzyl-1,4-dioxa-8,12-diazadispiro[4.0.4⁶.4⁵]tetradecane-12-carboxylate (p19, 2.25 g, 5.79 mmol) in MeOH (30 mL) ammonium formate (2.19 g, 34.75 mmol) and Pd/C (1.10 g) were added at RT then the mixture was stirred under reflux for 1 h. The mixture was cooled down to RT and filtered through a pad of celite washing with MeOH. Solvent was eliminated under reduced pressure affording tert-butyl 1,4-dioxa-8,12-diazadispiro[4.0.4⁶.4⁵]tetradecane-12-carboxylate (p20, 1.56 g, y=90%), as colorless oil.

MS (ES) (m/z): 299.2 [M+H]⁺.

### Preparation 21: tert-butyl 8-[bis(4-fluorophenyl)methyl]-1,4-dioxa-8,12-diazadispiro[4.0.4⁶.4⁵]tetradecane-12-carboxylate (P21)

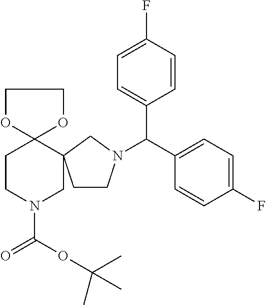 [SMILES: CC(C)(C)OC(=O)N1CCC2(OCCO2)C2(CCN(C(c3ccc(F)cc3)c3ccc(F)cc3)C2)C1]

Chlorobis(4-fluorophenyl)methane (0.104 mL, 0.562 mmol) was added to a stirred mixture of tert-butyl 1,4-dioxa-8,12-diazadispiro[4.0.4⁶.4⁵]tetradecane-12-carboxylate (p20, 0.140 g, 0.469 mmol) and K₂CO₃ (0.162 g, 1.17 mmol) in Acetonitrile (5 mL). The mixture was stirred for 2 hrs at reflux. The solution was filtered washing with EtOAc. The residue was purified by FC on silica gel (eluent: cHex to cHex/EtOAc 80/20) affording tert-butyl 8-[bis(4-fluorophenyl)methyl]-1,4-dioxa-8,12-diazadispiro[4.0.4⁶.4⁵]tetradecane-12-carboxylate (p21, 140 mg, y=59%) as white foam.

MS (ES) (m/z): 501.3 [M+H]⁺.

### Example 6: 8-[bis(4-fluorophenyl)methyl]-1,4-dioxa-8,12-diazadispiro[4.0.4⁶.4⁵]tetradecane (E6)

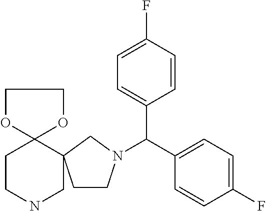 [SMILES: Fc1ccc(C(c2ccc(F)cc2)N2CCC3(CNCCC34OCCO4)C2)cc1]

To a solution of tert-butyl 8-[bis(4-fluorophenyl)methyl]-1,4-dioxa-8,12-diazadispiro[4.0.4⁶.4⁵]tetradecane-12-carboxylate (p21, 1 g, 2.99 mmol) in DCM (35 mL) TFA (4 mL) was added at RT then the solution was stirred for 1 h. The solution was evaporated and charged on SCX cartridge washing with MeOH and eluting with 1N NH₃ in MeOH affording 8-[bis(4-fluorophenyl)methyl]-1,4-dioxa-8,12-diazadispiro[4.0.4⁶.4⁵]tetradecane (E6, 1 g, y=83%) as pale yellow foam.

MS (ES) (m/z): 401.2 [M+H]⁺.

¹H NMR (CHLOROFORM-d): δ ppm 7.34-7.46 (m, 4H), 6.97 (td, 4H), 4.18 (s, 1H), 3.90-4.06 (m, 4H), 2.78-2.96 (m, 4H), 2.66 (td, 1H), 2.49 (d, 1H), 2.34 (d, 1H), 2.26 (q, 1H), 1.87-2.00 (m, 1H), 1.50-1.56 (m, 2H), 1.37-1.49 (m, 1H)

### Preparation 22: benzyl 8-[bis(4-fluorophenyl)methyl]-1,4-dioxa-8,12-diazadispiro[4.0.4⁶.4⁵]tetradecane-12-carboxylate (P22)

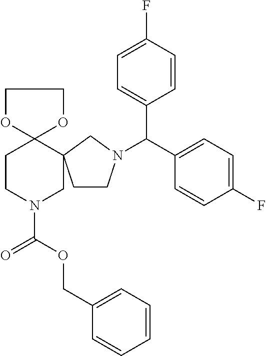 [SMILES: O=C(OCc1ccccc1)N1CCC2(OCCO2)C2(CCN(C(c3ccc(F)cc3)c3ccc(F)cc3)C2)C1]

TEA (0.87 mL, 6.22 mmol) was added to a stirred solution of 8-[bis(4-fluorophenyl)methyl]-1,4-dioxa-8,12-diazadispiro[4.0.4⁶.4⁵]tetradecane (E6, 1 g, 2.49 mmol) in DCM (30 mL); the solution was cooled at 0° C. and benzyl chloroformate (0.43 mL, 2.99 mmol) was added dropwise. The resulting mixture was stirred at RT for 1 h. The mixture was washed with NaHCO₃ (15 mL) and 1N HCl (15 mL), dried and concentrated under vacuum; residue was charged on SCX cartridge washing with MeOH and eluting with 2M NH₃ in MeOH to give benzyl 8-[bis(4-fluorophenyl)methyl]-1,4-dioxa-8,12-diazadispiro[4.0.4⁶.4⁵]tetradecane-12-carboxylate (p22, 1.21 g, y=91%) as white foam.

MS (ES) (m/z): 535.3 [M+H]⁺.

### Example 7 and Example 8: (5R or 5S)-2-[bis(4-fluorophenyl)methyl]-2,7-diazaspiro[4.5]decan-10-one (E7, Enantiomer 1) and (5S or 5R)-2-[bis(4-fluorophenyl)methyl]-2,7-diazaspiro[4.5]decan-10-one (E8, Enantiomer 2)

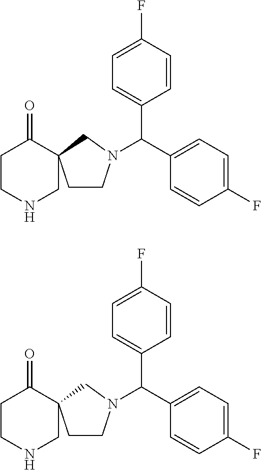 [SMILES: O=C1CCNC[C@@]12CCN(C(c1ccc(F)cc1)c1ccc(F)cc1)C2 ; O=C1CCNC[C@]12CCN(C(c1ccc(F)cc1)c1ccc(F)cc1)C2]

Step a

To a solution of benzyl 8-[bis(4-fluorophenyl)methyl]-1,4-dioxa-8,12-diazadispiro[4.0.4⁶.4⁵]tetradecane-12-carboxylate (p22, 1.21 g, 2.26 mmol) in DCM (50 mL) HClO₄ 70% (2.26 mL) was added and the reaction mixture was stirred at RT for 3 hrs. Then pH was adjusted to ˜9 using Na₂CO₃ and the product was extracted with DCM (20 mL×3). The organic phase was dried and concentrated under reduced pressure affording 2-[bis(4-fluorophenyl)methyl]-2,7-diazaspiro[4.5]decan-10-one (1 g, 70% purity) as white foam.

Step b

2-[bis(4-fluorophenyl)methyl]-2,7-diazaspiro[4.5]decan-10-one (from step a, racemic, 1 g) was separated into single enantiomers by preparative chiral HPLC, affording (5R or 5S)-2-[bis(4-fluorophenyl)methyl]-2,7-diazaspiro[4.5]decan-10-one (E7, Enantiomer 1, 200 mg) and (5S or 5R)-2-[bis(4-fluorophenyl)methyl]-2,7-diazaspiro[4.5]decan-10-one (E8, Enantiomer 2, 200 mg).

Preparative Chromatography:

Column: Chiralpak AD-H (25×3.0 cm), 5μ

Mobile phase: n-Hexane/Ethanol 70/30% v/v

Flow rate (mL/min): 33 mL/min

DAD detection: 220 nm

Injection: 37.5 mg/injection

E7, Enantiomer 1: ret. time (min) 12.1 100% ee

E8, Enantiomer 2: ret. time (min) 16.6 >99% ee

MS (ES) (m/z): 357.20 [M+H]⁺.

¹H NMR (CHLOROFORM-d): δ ppm 7.29 (m, 4H), 6.89 (t, 4H), 4.09 (s, 1H), 3.03 (m, 2H), 2.97 (d, 1H), 2.85 (d, 1H), 2.56 (m, 1H), 2.47 (m, 2H), 2.37 (m, 1H), 2.26 (d, 3H), 1.51 (br. S., 1H).

### Example 9: (5R or 5S)-2-[bis(4-fluorophenyl)methyl]-2,7-diazaspiro[4.5]decan-10-one dihydrochloride salt (E9, Enantiomer 1)

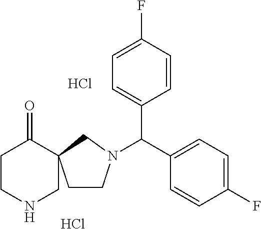 [SMILES: O=C1CCNC[C@@]12CCN(C(c1ccc(F)cc1)c1ccc(F)cc1)C2 ; Cl ; Cl]

1M HCl in Et₂O (1.25 mL, 1.25 mmol) was added dropwise to a stirred solution of (5R or 5S)-2-[bis(4-fluorophenyl)methyl]-2,7-diazaspiro[4.5]decan-10-one (E7, Enantiomer 1, 200 mg, 0.561 mmol) in Et₂O (2 mL); the solution was stirred at RT for 1 h. The mixture was concentrated to dryness to obtain (5R or 5S)-2-[bis(4-fluorophenyl)methyl]-2,7-diazaspiro[4.5]decan-10-one dihydrochloride salt (E9, Enantiomer 1, 240 mg, y=99%) as white solid.

MS (ES) (m/z): 357.22 [M+H]⁺.

### Example 10: (5S or 5R)-2-[bis(4-fluorophenyl)methyl]-2,7-diazaspiro[4.5]decan-10-one dihydrochloride salt (E10, Enantiomer 2)

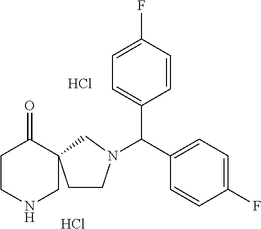 [SMILES: O=C1CCNC[C@]12CCN(C(c1ccc(F)cc1)c1ccc(F)cc1)C2 ; Cl ; Cl]

1M HCl in Et₂O (1.25 mL, 1.25 mmol) was added dropwise to a stirred solution of (5S or 5R)-2-[bis(4-fluorophenyl)methyl]-2,7-diazaspiro[4.5]decan-10-one (E8, Enantiomer 2, 200 mg, 0.561 mmol) in Et₂O (2 mL); the solution was stirred at RT for 1 h. The mixture was concentrated to dryness to obtain (5S or 5R)-2-[bis(4-fluorophenyl)methyl]-2,7-diazaspiro[4.5]decan-10-one dihydrochloride salt (E10, Enantiomer 2, 240 mg, y=99%) as white solid.

MS (ES) (m/z): 357.21 [M+H]⁺.

### Example 11 and example 12: 2-[bis(4-fluorophenyl)methyl]-2,7-diazaspiro[4.5]decan-10-ol (E11 and E12)

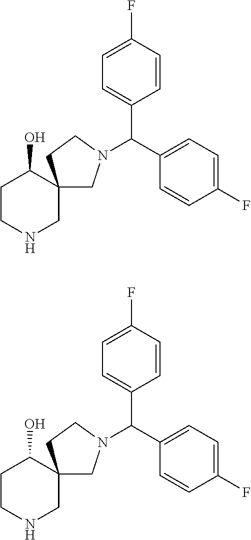 [SMILES: O[C@@H]1CCNC[C@]12CCN(C(c1ccc(F)cc1)c1ccc(F)cc1)C2 ; O[C@H]1CCNC[C@]12CCN(C(c1ccc(F)cc1)c1ccc(F)cc1)C2]

To a stirred solution of (5S or 5R)-2-[bis(4-fluorophenyl)methyl]-2,7-diazaspiro[4.5]decan-10-one (E7, single enantiomer: enantiomer 1, 13.5 mg, 0.0378 mmol) in MeOH (1 mL) NaBH₄ (3 mg, 0.076 mmol) was added. The reaction was stirred at RT for 1 h and then MeOH was removed. The residue was loaded on SCX cartridge washing with MeOH and eluting with 1M NH₃ in MeOH to afford 2-[bis(4-fluorophenyl)methyl]-2,7-diazaspiro[4.5]decan-10-ol (14 mg, mixture of diastereoisomers) that was submitted to prep HPLC to separate diastereoisomers.

Preparative HPLC Conditions and Results:

| Column | Chiralpak AD-H (25 × 2.0 cm), 5μ | |
|---|---|---|
| Mobile phase | n-Hexane/(Ethanol + 0.1% isopropylamine) | |
| | 70/30% v/v | |
| Flow rate (mL/min) | 15 | mL/min |
| DAD detection | 220 | nm |
| Loop | 1000 | μL |
| Total amount | 14 | mg |

| | |
|---|---|
| Solubilization | 14 mg in 1 ml EtOH = 14 mg/ml |
| Injection | 14 mg (each injection) |

Affording:

2-[bis(4-fluorophenyl)methyl]-2,7-diazaspiro[4.5]decan-10-ol (Relative stereochemistry syn) (E11, 4.7 mg, y=34%) 100% ed

MS (ES) (m/z): 359.2 [M+H]⁺.

¹H NMR (CHLOROFORM-d): δ ppm 7.27 (td, 4H), 6.90 (q, 4H), 4.80 (br. s, 1H), 4.05 (s, 1H), 3.41 (dd, 1H), 3.07 (d, 1H), 2.91 (d, 1H), 2.80 (m, 1H), 2.75 (d, 1H), 2.51 (m, 2H), 2.10 (q, 1H), 1.94 (m, 2H), 1.84 (dd, 1H), 1.48 (m, 1H), 1.35 (m, 1H)

2-[bis(4-fluorophenyl)methyl]-2,7-diazaspiro[4.5]decan-10-ol (Relative stereochemistry anti) (E12, 4.2 mg, y=31%) 100% ed

MS (ES) (m/z): 359.2 [M+H]⁺.

¹H NMR (CHLOROFORM-d): δ ppm 7.26 (m, 4H), 6.91 (q, 4H), 4.84 (br. s, 1H), 4.01 (s, 1H), 3.53 (dd, 1H), 2.94 (d, 1H), 2.87 (m, 1H), 2.65 (d, 1H), 2.61 (d, 1H), 2.46 (td, 1H), 2.40 (d, 1H), 2.10 (m, 2H), 1.91 (d, 1H), 1.70 (m, 1H), 1.51 (m, 1H), 1.39 (m, 1H)

### Example 13 and Example 14: 2-[bis(4-fluorophenyl)methyl]-2,7-diazaspiro[4.5]decan-10-ol (E13 and E14)

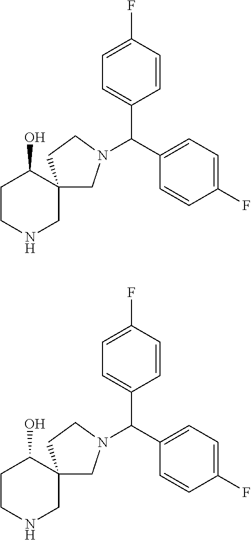 [SMILES: O[C@@H]1CCNC[C@@]12CCN(C(c1ccc(F)cc1)c1ccc(F)cc1)C2 ; O[C@H]1CCNC[C@@]12CCN(C(c1ccc(F)cc1)c1ccc(F)cc1)C2]

To a stirred solution of (5S or 5R)-2-[bis(4-fluorophenyl)methyl]-2,7-diazaspiro[4.5]decan-10-one (E8, 13 mg, 0.036 mmol) in MeOH (1 mL) NaBH₄ (3 mg, 0.073 mmol) was added. The reaction was stirred at RT for 1 h and then MeOH was removed. The residue was charged on SCX cartridge washing with MeOH and eluting with 1M NH₃ in MeOH to afford 2-[bis(4-fluorophenyl)methyl]-2,7-diazaspiro[4.5]decan-10-ol (13 mg, mixture of diastereoisomers) that was submitted to prep HPLC to separate diastereoisomers.

Preparative HPLC Conditions and Results:

| Column | Chiralpak AD-H (25 × 2.0 cm), 5μ | |
|---|---|---|
| Mobile phase | n-Hexane/(Ethanol + 0.1% isopropylamine) | |
| | 70/30% v/v | |
| Flow rate (mL/min) | 13 | mL/min |
| DAD detection | 220 | nm |
| Loop | 3000 | μL |
| Total amount | 14 | mg |

| | |
|---|---|
| Solubilization | 14 mg in 3 ml EtOH = 4.7 mg/ml |
| Injection | 14 mg (each injection) |

Affording:

2-[bis(4-fluorophenyl)methyl]-2,7-diazaspiro[4.5]decan-10-ol (Relative stereochemistry anti) (E13, 3 mg, y=23%) 100% ed

MS (ES) (m/z): 359.2 [M+H]⁺.

¹H NMR (CHLOROFORM-d): δ ppm 7.26 (m, 4H), 6.91 (q, 4H), 4.84 (br. s, 1H), 4.01 (s, 1H), 3.53 (dd, 1H), 2.94 (d, 1H), 2.87 (m, 1H), 2.65 (d, 1H), 2.61 (d, 1H), 2.46 (td, 1H), 2.40 (d, 1H), 2.10 (m, 2H), 1.91 (d, 1H), 1.70 (m, 1H), 1.51 (m, 1H), 1.39 (m, 1H)

2-[bis(4-fluorophenyl)methyl]-2,7-diazaspiro[4.5]decan-10-ol (Relative stereochemistry syn) (E14, 2.5 mg, y=19%) 95.4% ed

MS (ES) (m/z): 359.2 [M+H]⁺.

¹H NMR (CHLOROFORM-d): δ ppm 7.27 (td, 4H), 6.90 (q, 4H), 4.80 (br. s, 1H), 4.05 (s, 1H), 3.41 (dd, 1H), 3.07 (d, 1H), 2.91 (d, 1H), 2.80 (m, 1H), 2.75 (d, 1H), 2.51 (m, 2H), 2.10 (q, 1H), 1.94 (m, 2H), 1.84 (dd, 1H), 1.48 (m, 1H), 1.35 (m, 1H)

### Example 15: (5S or 5R,10E)-2-[bis(4-fluorophenyl)methyl]-N-methoxy-2,7-diazaspiro[4.5]decan-10-imine (E15)

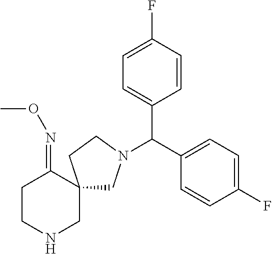 [SMILES: CO/N=C1\CCNC[C@]12CCN(C(c1ccc(F)cc1)c1ccc(F)cc1)C2]

To a solution of (5S or 5R)-2-[bis(4-fluorophenyl)methyl]-2,7-diazaspiro[4.5]decan-10-one dihydrochloride (E8, single enantiomer: enantiomer 2, 30 mg, 0.07 mmol) in a mixture EtOH/water (1/1 mL) methylhydroxylamine hydrochloride (13 mg, 0.15 mmol) was added followed by 0.23 mL of NaOH 1M aq sol. The mixture was refluxed overnight. The mixture was then cooled down to RT and then solvent was eliminated under reduced pressure; the residue was dissolved again in EtOH (1 mL), sodium acetate was added (13 mg) and the mixture refluxed overnight. The day after solvent was eliminated under reduced pressure, the residue was triturated with Acetonitrile affording (5S or 5R,10E)-2-[bis(4-fluorophenyl)methyl]-N-methoxy-2,7-diazaspiro[4.5]decan-10-imine (E15, 19 mg, y=64%)

MS (ES) (m/z): 386.25 [M+H]⁺.

¹H NMR (CHLOROFORM-d): δ ppm 7.39 (br. s., 4H), 6.91-7.06 (m, 4H), 4.23 (br. s., 1H) 3.87 (br. s., 3H), 2.81-3.03 (m, 4H), 2.66 (br. s., 2H), 2.50 (br. s., 3H), 2.35 (d, 1H), 2.26 (br. s., 1H)

### Example 16: N-[(5S or 5R,10E)-2-[bis(4-fluorophenyl)methyl]-2,7-diazaspiro[4.5]decan-10-ylidene]hydroxylamine (E16)

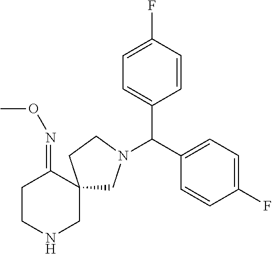 [SMILES: CO/N=C1\CCNC[C@]12CCN(C(c1ccc(F)cc1)c1ccc(F)cc1)C2]

To a solution of (5S or 5R)-2-[bis(4-fluorophenyl)methyl]-2,7-diazaspiro[4.5]decan-10-one dihydrochloride (E8, single enantiomer: enantiomer 2, 30 mg, 0.07 mmol) in a mixture EtOH/water (1/1 mL) hydroxylamine hydrochloride (10 mg, 0.14 mmol) was added followed by 0.21 mL of NaOH 1M aq sol. The mixture was refluxed for 1 h. Reaction was cooled down to RT, concentrated under reduced pressure and triturated with Acetonitrile affording N-[(5S or 5R,10E)-2-[bis(4-fluorophenyl)methyl]-2,7-diazaspiro[4.5]decan-10-ylidene]hydroxylamine (E16, 24 mg, y=92%) as white solid.

MS (ES) (m/z): 372.22 [M+H]⁺.

¹H NMR (CHLOROFORM-d): δ ppm 7.39 (br. s., 4H), 6.92-7.03 (m, 4H), 4.20 (br. s., 1H), 2.93 (s, 4H), 2.52-2.73 (m, 4H), 2.46 (br. s., 1H), 2.20-2.35 (m, 2H).

### Preparation 23: (5S or 5R)-tert-butyl 2-[bis(4-fluorophenyl)methyl]-10-oxo-2,7-diazaspiro[4.5]decane-7-carboxylate (P23)

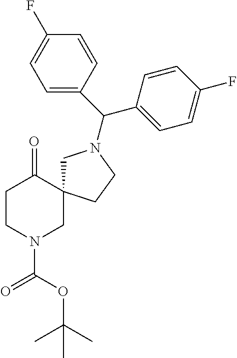 [SMILES: CC(C)(C)OC(=O)N1CCC(=O)[C@@]2(CCN(C(c3ccc(F)cc3)c3ccc(F)cc3)C2)C1]

(5S or 5R)-2-[bis(4-fluorophenyl)methyl]-2,7-diazaspiro[4.5]decan-10-one (E8, single enantiomer: enantiomer 2, 90 mg, 0.253 mmol) was dissolved in H₂O (3 mL) at RT then cooled down to 0° C. Na₂CO₃ (26.11 mg, 0.258 mmol) was added followed by the drop-wise addition of a solution of Boc₂O (55.11 mg, 0.253 mmol) in THF (3 mL). The mixture was stirred at the same temperature for 1 h then worked up extracting with EtOAc. The organic phase was washed with brine, dried and concentrated under reduced pressure to obtain (5S or 5R)-tert-butyl 2-[bis(4-fluorophenyl)methyl]-10-oxo-2,7-diazaspiro[4.5]decane-7-carboxylate (p23, 109 mg, y=94%) as white foam.

MS (ES) (m/z): 457.26 [M+H]⁺.

### Preparation 24: mixture of (5S or 5R)-tert-butyl 2-[bis(4-fluorophenyl)methyl]-10,10-difluoro-2,7-diazaspiro[4.5]decane-7-carboxylate and (5S or 5R)-tert-butyl 2-[bis(4-fluorophenyl)methyl]-10-fluoro-2,7-diazaspiro[4.5]dec-9-ene-7-carboxylate (P24)

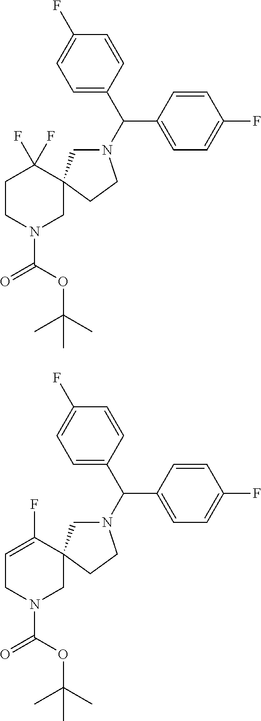

(5S or 5R)-tert-butyl 2-[bis(4-fluorophenyl)methyl]-10-oxo-2,7-diazaspiro[4.5]decane-7-carboxylate (p23, 109 mg, 0.239 mmol) was dissolved in anhydrous DCM (8 mL). The solution was cooled down to −10° C. under nitrogen atmosphere and diethylaminosulfur trifluoride (DAST) (0.1 mL, 0.764 mmol) was added dropwise. The reaction mixture was allowed to warm to RT and stirred overnight. The mixture was quenched with cold water. The separated aqueous layer was extracted with DCM and the combined organic layers were dried. After filtration, the solvent was concentrated and crude material was purified by FC on silica gel (eluent: Cy to Cy/AcOEt 90/10) to give a mixture of (5S or 5R)-tert-butyl 2-[bis(4-fluorophenyl)methyl]-10,10-difluoro-2,7-diazaspiro[4.5]decane-7-carboxylate and (5S or 5R)-tert-butyl 2-[bis(4-fluorophenyl)methyl]-10-fluoro-2,7-diazaspiro[4.5]dec-9-ene-7-carboxylate (p24, 64 mg) in a 1:1 ratio.

MS (ES) (m/z): 459.24 [M+H]⁺, 479.21 [M+H]⁺.

### Example 17 and Example 18: (5S or 5R)-2-[bis(4-fluorophenyl)methyl]-10,10-difluoro-2,7-diazaspiro[4.5]decane (E17) and (5S or 5R)-2-[bis(4-fluorophenyl)methyl]-10-fluoro-2,7-diazaspiro[4.5]dec-9-ene (E18)

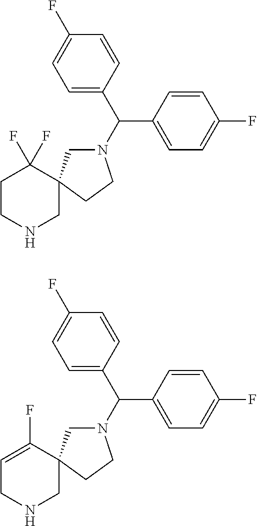 [SMILES: Fc1ccc(C(c2ccc(F)cc2)N2CC[C@]3(CNCCC3(F)F)C2)cc1 ; FC1=CCNC[C@]12CCN(C(c1ccc(F)cc1)c1ccc(F)cc1)C2]

TFA (0.5 mL) was added to a solution of a mixture of (5S or 5R)-tert-butyl 2-[bis(4-fluorophenyl)methyl]-10,10-difluoro-2,7-diazaspiro[4.5]decane-7-carboxylate and (5S or 5R)-tert-butyl 2-[bis(4-fluorophenyl)methyl]-10-fluoro-2,7-diazaspiro[4.5]dec-9-ene-7-carboxylate (p24, 64 mg) in 3 mL of DCM. The mixture was stirred at RT for 30 min and then the solvent was removed under reduced pressure. The residue was charged on SCX cartridge washing with meOH and eluting with 1M NH₃ in MeOH affording a mixture of (5S or 5R)-2-[bis(4-fluorophenyl)methyl]-10,10-difluoro-2,7-diazaspiro[4.5]decane and (5S or 5R)-2-[bis(4-fluorophenyl)methyl]-10-fluoro-2,7-diazaspiro[4.5]dec-9-ene in a ratio ˜1:1 (49 mg). Combined batches from similar preparations (60 mg) were submitted to prep HPLC:

LC/MS Conditions:

Columns: XSelect CSH Prep. C18 5 μm OBD 30×100 mm at RT

Injection loop 1 mL

Solvents: A=H2O+0.1% HCOOH
B=Acetonitrile+0.1% HCOOH

Gradient:

| Time (min) | Flow Rate (mL/min) | % A | % B | Curve |
|---|---|---|---|---|
| initial | 40.0 | 97.0 | 3.0 | — |
| 10.0 | 40.0 | 50.0 | 50.0 | 6 |
| 10.5 | 40.0 | 0.0 | 100.0 | 6 |
| 14.5 | 40.0 | 0.0 | 100.0 | 6 |
| 15.0 | 40.0 | 97.0 | 3.0 | 6 |

The curve parameter followed Waters definition (6=linear, 11=step).

Acquisition stop time: 16.0 min

UV Conditions:

UV detection range: 210 nm to 400 nm

Acquisition rate: 1.0 spectra/s

MS Conditions:

Ionisation mode: Positive Electrospray (ES⁺⁾

Scan Range: ES⁺/ES⁻ 100 to 900 AMU

affording, after solvent removal, the corresponding formic amides.

Each one of the latter was dissolved in MeOH, treated with HCl 2M in Et₂O (0.2 mL) and stirred at RT for 18 hrs. Solvent was eliminated under reduced pressure and the compounds were loaded on SCX cartridges washing with MeOH and eluting with NH₃ 1M in MeOH affording:

(5S or 5R)-2-[bis(4-fluorophenyl)methyl]-10,10-difluoro-2,7-diazaspiro[4.5]decane (E17, 13 mg)

MS (ES) (m/z): 379.23 [M+H]⁺.

¹H NMR (CHLOROFORM-d): δ ppm 7.41 (br. s., 4H), 6.99 (t, 4H), 5.24 (d, 1H), 4.21 (br. s., 1H), 3.25-3.45 (m, 2H), 3.12 (d, 1H), 2.83 (s, 1H), 2.86 (s, 1H), 2.72 (br. s., 1H), 2.60 (d, 1H), 2.44 (d, 1H), 2.30 (d, 1H), 2.14 (br. s., 1H)

(5S or 5R)-2-[bis(4-fluorophenyl)methyl]-10-fluoro-2,7-diazaspiro[4.5]dec-9-ene (E18, 15 mg)

MS (ES) (m/z): 359.19 [M+H]⁺.

¹H NMR (CHLOROFORM-d): δ ppm 7.40 (br. s., 4H), 6.88-7.06 (m, 4H), 4.20 (br. s., 1H), 2.85-3.06 (m, 4H), 2.67 (br. s., 1H), 2.52 (br. s., 2H), 2.33 (d, 2H), 2.08 (br. s., 1H), 1.86-2.03 (m, 1H), 1.80 (br. s., 1H)

### Preparation 25: tert-butyl 8-[bis(4-chlorophenyl)methyl]-1,4-dioxa-8,12-diazadispiro[4.0.4⁶.4⁵]tetradecane-12-carboxylate (P25)

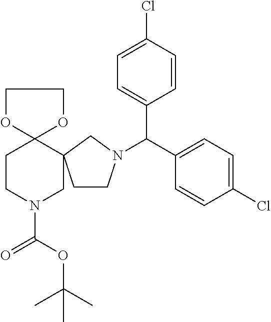 [SMILES: CC(C)(C)OC(=O)N1CCC2(OCCO2)C2(CCN(C(c3ccc(Cl)cc3)c3ccc(Cl)cc3)C2)C1]

1-chloro-4-[chloro(4-chlorophenyl)methyl]benzene (160 mg, 0.6 mmol) was added to a stirred mixture of tert-butyl 8-benzyl-1,4-dioxa-8,12-diazadispiro[4.0.4⁶.4⁵]tetradecane-12-carboxylate (p20, 150 mg, 0.5 mmol) and K₂CO₃ (172 mg, 1.25 mmol) in Acetonitrile (4 mL). The mixture was stirred for 18 hrs at reflux. The solution was filtered washing with EtOAc and evaporated. The crude material was purified by FC on silica gel (eluent: EtOAc to EtOAc/MeOH 80/20) affording tert-butyl 8-[bis(4-chlorophenyl)methyl]-1,4-dioxa-8,12-diazadispiro[4.0.4⁶.4⁵]tetradecane-12-carboxylate (p25, 160 mg, y=60%) as yellow oil.

MS (ES) (m/z): 533.2 [M]⁺.

### Preparation 26: 8-[bis(4-chlorophenyl)methyl]-1,4-dioxa-8,12-diazadispiro[4.0.46.45]tetradecane (P26)

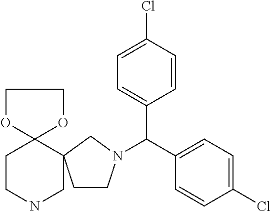 [SMILES: Clc1ccc(C(c2ccc(Cl)cc2)N2CCC3(CNCCC34OCCO4)C2)cc1]

To a solution of tert-butyl 8-[bis(4-chlorophenyl)methyl]-1,4-dioxa-8,12-diazadispiro[4.0.4⁶.4⁵]tetradecane-12-carboxylate (p25, 160 mg, 0.299 mmol) in DCM (3 mL) TFA (0.5 mL) was added at RT then the solution was stirred for 1 h. The solution was evaporated and loaded on SCX cartridge eluting with 1N NH₃ in MeOH affording 8-[bis(4-chlorophenyl)methyl]-1,4-dioxa-8,12-diazadispiro[4.0.4⁶.4⁵]tetradecane (p26, 120 mg, y=93%) as pale yellow foam.

MS (ES) (m/z): 433.2 [M].

### Preparation 27: benzyl 8-[bis(4-chlorophenyl)methyl]-1,4-dioxa-8,12-diazadispiro[4.0.4⁶.4⁵]tetradecane-12-carboxylate (P27)

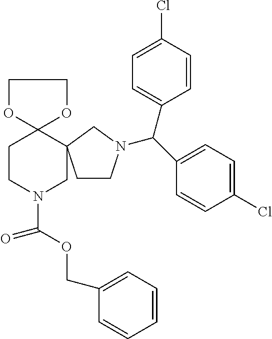 [SMILES: O=C(OCc1ccccc1)N1CCC2(OCCO2)C2(CCN(C(c3ccc(Cl)cc3)c3ccc(Cl)cc3)C2)C1]

TEA (0.096 mL, 0.69 mmol) was added to a stirred solution of 8-[bis(4-chlorophenyl)methyl]-1,4-dioxa-8,12-diazadispiro[4.0.4⁶.4⁵]tetradecane (p26, 120 mg, 0.277 mmol) in DCM (5 mL); the solution was cooled at 0° C. and benzyl chloroformate (0.047 mL, 0.33 mmol) was added dropwise. The resulting mixture was stirred at RT for 1 h. The mixture was then washed with NaHCO₃ (5 mL) and 1N HCl (5 mL), dried, concentrated under vacuum, and loaded on SCX cartridge washing with MeOH and eluting with 1M NH₃ in MeOH to give benzyl 8-[bis(4-chlorophenyl)methyl]-1,4-dioxa-8,12-diazadispiro[4.0.4⁶.4⁵]tetradecane-12-carboxylate (p27, 155 mg, y=98%) as yellow oil.

MS (ES) (m/z): 567.1 [M]⁺.

### Example 19: 2-[bis(4-chlorophenyl)methyl]-2,7-diazaspiro[4.5]decan-10-one (E19)

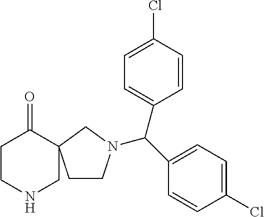 [SMILES: O=C1CCNCC12CCN(C(c1ccc(Cl)cc1)c1ccc(Cl)cc1)C2]

To a solution of benzyl 8-[bis(4-chlorophenyl)methyl]-1,4-dioxa-8,12-diazadispiro[4.0.4⁶.4⁵]tetradecane-12-carboxylate (p27, 155 mg, 0.273 mmol) in DCM (3 mL) HClO₄ 70% (0.270 mL) was added and stirred at RT for 3 hrs. Then pH was adjusted to ˜9 using Na₂CO₃, the product was extracted with DCM (5 mL×3). The organic phase was dried, evaporated and purified by FC on NH column (eluent: cHex to cHex/EtOAc 60/40) affording 2-[bis(4-chlorophenyl)methyl]-2,7-diazaspiro[4.5]decan-10-one (E19, 8 mg, y=7%) as white foam.

MS (ES) (m/z): 377.14 [M+H]⁺.

¹H NMR (CHLOROFORM-d): δ ppm 7.35 (d, 4H), 7.25 (dd, 4H), 4.16 (s, 1H), 3.01-3.18 (m, 3H), 2.88-2.97 (m, 1H), 2.51-2.69 (m, 3H), 2.46 (dt, 1H), 2.27-2.40 (m, 3H), 1.53-1.63 (m, 1H)

### Preparation 29: Ethyl 1-benzyl-3-oxo-4-(prop-2-en-1-yl)piperidine-4-carboxylate (P29)

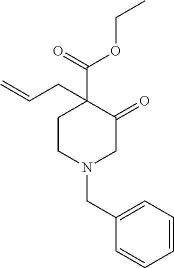 [SMILES: C=CCC1(C(=O)OCC)CCN(Cc2ccccc2)CC1=O]

A mixture of potassium tert-butoxide (3.77 g, 33.58 mmol) in THF (100 mL) was stirred at RT for 0.5 h. The resulting milky solution was cooled to 0° C., and then Ethyl 1-benzyl-3-oxopiperidine-4-carboxylate hydrochloride (5 g, 16.79 mmol) was added portion wise keeping the internal temperature below 5° C. The mixture was then warmed to RT and further stirred for 1 h, resulting in a yellow solution. After cooling to 0° C., allyl bromide (1.6 mL, 18.47 mmol) was added dropwise. The reaction mixture was warmed to RT and stirred overnight. The reaction solution was cooled to 0° C., and 50 mL of saturated NH₄Cl solution was added. After extraction and phase separation, the aqueous phase was extracted twice with 100 mL of EA. The combined organic phases were washed with 100 mL of saturated NaCl solution and dried; the solvent was evaporated under reduced pressure and the obtained crude material was purified by FC on silica gel (eluent: Cy to Cy/AcOEt 80/20) to give ethyl 1-benzyl-3-oxo-4-(prop-2-en-1-yl)piperidine-4-carboxylate (p29, 4.23 g, y=84%) as a yellow oil.

MS (ES) (m/z): 302.23 [M+H]⁺.

### Preparation 30: Ethyl 3-oxo-4-(prop-2-en-1-yl)piperidine-4-carboxylate (P30)

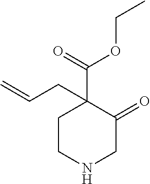

To a solution of Ethyl 1-benzyl-3-oxo-4-(prop-2-en-1-yl)piperidine-4-carboxylate (p29, 4.23 g, 14.04 mmol) in DCE (100 mL) ACE-Cl (4.6 mL, 42.12 mmol) was added dropwise. The mixture was heated to reflux and stirred for 2 hrs. Further ACE-Cl (10 mL) was added and the mixture was stirred at reflux overnight. Solvent was evaporated; residue was dissolved in MeOH and refluxed for 1.5 h. The solvent was evaporated and the obtained crude material was purified by FC on silica gel (eluent: DCM/MeOH/2M NH₃ in MeOH from 98/2/0 to 80/15/5) to give Ethyl 3-oxo-4-(prop-2-en-1-yl)piperidine-4-carboxylate (p30, 2.87 g, y=96%).

MS (ES) (m/z): 212.16 [M+H]⁺.

### Preparation 31: 1-benzyl 4-ethyl 3-oxo-4-(prop-2-en-1-yl)piperidine-1,4-dicarboxylate (P31)

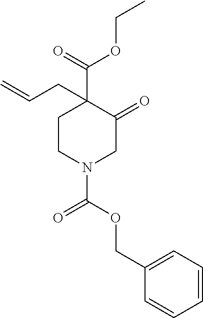 [SMILES: C=CCC1(C(=O)OCC)CCN(C(=O)OCc2ccccc2)CC1=O]

To a solution of Ethyl 3-oxo-4-(prop-2-en-1-yl)piperidine-4-carboxylate (p30, 2.87 g, 13.58 mmol) in DCM (50 mL) at 0° C., Benzyl chloroformate (3.86 mL 27.16 mmol) and DIPEA (4.73 mL, 27.16 mmol) were added dropwise. Once the addition was complete, the reaction mixture was allowed to reach RT and left stirring at that temperature for 2 hrs. It was quenched with water and phases were separated. Aqueous phase was back extracted with DCM. Organic layers were combined, dried and concentrated. The obtained crude material was purified by FC on silica gel (eluent: Cy/EtOAc from 95/5 to 80/20) to give 1-benzyl 4-ethyl 3-oxo-4-(prop-2-en-1-yl)piperidine-1,4-dicarboxylate (p31, 3.85 g, y=82%, purity: 65% by UV a/a)

MS (ES) (m/z): 346.2 [M+H]⁺.

### Preparation 32: 7-benzyl 10-ethyl 10-(prop-2-en-1-yl)-1,4-dioxa-7-azaspiro[4.5]decane-7,10-dicarboxylate (P32)

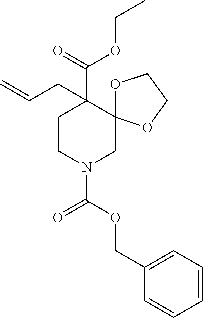 [SMILES: C=CCC1(C(=O)OCC)CCN(C(=O)OCc2ccccc2)CC12OCCO2]

A mixture of 1-benzyl 4-ethyl 3-oxo-4-(prop-2-en-1-yl)piperidine-1,4-dicarboxylate (p31, 4 g, 11.15 mmol), ethylene glycol (6.22 mL, 111.5 mmol) and p-Toluensulfonic acid monohydrate (317.77 mg, 1.67 mmol) in dry Toluene (10 mL) was heated under reflux overnight using a Dean-Stark apparatus. The mixture was cooled down to RT and concentrated under vacuum. The residue was dissolved in Et₂O and washed with water. After evaporation of the organic phase, the crude material was purified by FC on silica gel (eluent: cHex/EtOAc from 9/1 to 6/4) to afford 7-benzyl 10-ethyl 10-(prop-2-en-1-yl)-1,4-dioxa-7-azaspiro[4.5]decane-7,10-dicarboxylate (p32, 2.678 g, y=61.7%).

MS (ES) (m/z): 390.21 [M+H]⁺.

### Preparation 33: 7-benzyl 10-ethyl 10-(2-oxoethyl)-1,4-dioxa-7-azaspiro[4.5]decane-7,10-dicarboxylate (P33)

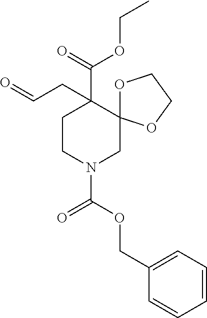 [SMILES: CCOC(=O)C1(CC=O)CCN(C(=O)OCc2ccccc2)CC12OCCO2]

1-benzyl 4-ethyl 3-oxo-4-(prop-2-en-1-yl)piperidine-1,4-dicarboxylate (p32, 2.678 g, 6.88 mmol) was dissolved in THF/H₂O (30+30 mL). To this stirred mixture a solution of OsO₄ 4% in water (3.5 mL, 0.55 mmol) was added over 30 seconds, the resulting mixture was stirred at RT for 5 min. NaIO₄ (3.68 g, 17.2 mmol) was added and the mixture stirred for 2 hrs. The mixture was partitioned between NaHCO₃ and Et₂O (×3). The organic phase was dried and evaporated. The crude material was purified by FC on silica gel (eluent: cHex/EtOAc from 8/2 to 6/4) to give 7-benzyl 10-ethyl 10-(2-oxoethyl)-1,4-dioxa-7-azaspiro[4.5]decane-7,10-dicarboxylate (p33, 2.03 g, y=75%) as a colourless oil.

MS (ES) (m/z): 392.18 [M+H]⁺.

### Preparation 34: benzyl 8-benzyl-7-oxo-1,4-dioxa-8,13-diazadispiro[4.0.4⁶.4⁵]tetradecane-13-carboxylate (P34)

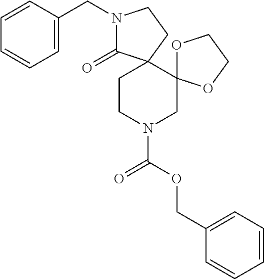 [SMILES: O=C(OCc1ccccc1)N1CCC2(CCN(Cc3ccccc3)C2=O)C2(C1)OCCO2]

To a solution of 7-benzyl 10-ethyl 10-(2-oxoethyl)-1,4-dioxa-7-azaspiro[4.5]decane-7,10-dicarboxylate (p33, 2.03 g, 5.19 mmol) and benzylamine (0.75 mL, 6.75 mmol) in THF (40 mL), Na(AcO)₃BH (2.2 g, 10.38 mmol) was added. The resulting mixture was stirred at RT overnight. The mixture was partitioned between NaHCO₃ and EtOAc. The organic phase was dried and evaporated. Crude material was purified by FC on silica gel (eluent: Cy/EtOAc from 8/2 to 5/5) to give benzyl 8-benzyl-7-oxo-1,4-dioxa-8,13-diazadispiro[4.0.4⁶.4⁵]tetradecane-13-carboxylate (p34, 1.98 g, y=87%) as a colourless oil.

MS (ES) (m/z): 437.3 [M+H]⁺.

### Preparation 35: 8-benzyl-1,4-dioxa-8,13-diazadispiro[4.0.4⁶.4⁵]tetradecan-7-one (P35)

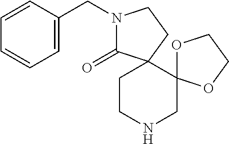 [SMILES: O=C1N(Cc2ccccc2)CCC12CCNCC21OCCO1]

Under a hydrogen atmosphere, a mixture of benzyl 8-benzyl-7-oxo-1,4-dioxa-8,13-diazadispiro[4.0.4⁶.4⁵]tetradecane-13-carboxylate (p34, 1.98 g, 4.54 mmol), 10% Pd/C (313 mg), and MeOH (25 mL) was stirred at RT for 1.5 h. The Pd/C was filtered off, the mixture was washed with MeOH, and the filtrate was concentrated under reduced pressure to obtain 8-benzyl-1,4-dioxa-8,13-diazadispiro[4.0.4⁶.4⁵]tetradecan-7-one (p35, 1.33 g, crude material), as a colorless oil, which was used in the next step without purification.

MS (ES) (m/z): 303.2 [M+H]⁺.

### Preparation 36: 8-benzyl-1,4-dioxa-8,13-diazadispiro[4.0.4⁶.4⁵]tetradecane (P36)

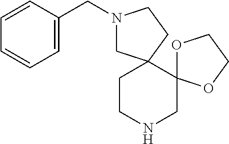 [SMILES: c1ccc(CN2CCC3(CCNCC34OCCO4)C2)cc1]

To a solution of 8-benzyl-1,4-dioxa-8,13-diazadispiro[4.0.4⁶.4⁵]tetradecan-7-one (p35, 1.33 g, 4.4 mmol) in THF (10 mL) 1M LiAlH₄ (6.6 mL, 6.6 mmol) was added dropwise at 0° C. The mixture was heated to reflux and stirred for 1.5 h. The reaction was quenched with Na₂SO₄*10H₂O, the mixture was filtered washing with EtOAc and the filtrate was concentrated under reduced pressure affording 8-benzyl-1,4-dioxa-8,13-diazadispiro[4.0.4⁶.4⁵]tetradecane (p36, 1.33 g, crude material) as a colorless oil, which was used in the next step without further purification.

MS (ES) (m/z): 289.2 [M+H]⁺.

### Preparation 37: tert-butyl 8-benzyl-1,4-dioxa-8,13-diazadispiro[4.0.4⁶.4⁵]tetradecane-13-carboxylate (P37)

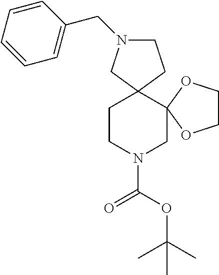 [SMILES: CC(C)(C)OC(=O)N1CCC2(CCN(Cc3ccccc3)C2)C2(C1)OCCO2]

8-benzyl-1,4-dioxa-8,13-diazadispiro[4.0.4⁶.4⁵]tetradecane (p36, 1.33 g, 4.6 mmol) was dissolved in water (12 mL) and cooled down to 0° C. Na₂CO₃ (466 mg, 4.6 mmol) was added followed by the dropwise addition of a solution of Boc₂O (1 g, 4.6 mmol) in THF (10 mL). The reaction mixture was stirred at 0° C. for 1 h, then it was extracted with EtOAc (×2). The organic phases were combined, washed with brine, dried and concentrated to give tert-butyl 8-benzyl-1,4-dioxa-8,13-diazadispiro[4.0.46.45]tetradecane-13-carboxylate (p37, 1.8 g) as a colorless oil.

MS (ES) (m/z): 389.3 [M+H]⁺.

### Preparation 38: tert-butyl 1,4-dioxa-8,13-diazadispiro[4.0.4⁶.4⁵]tetradecane-13-carboxylate (P38)

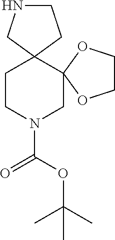 [SMILES: CC(C)(C)OC(=O)N1CCC2(CCNC2)C2(C1)OCCO2]

To a solution of tert-butyl 8-benzyl-1,4-dioxa-8,13-diazadispiro[4.0.46.45]tetradecane-13-carboxylate (p37, 1.8 g, 4.66 mmol) in MeOH (30 mL) ammonium formate (1.76 g, 27.9 mmol) and 10% Pd/C (0.5 g) were added. The reaction mixture was stirred under reflux for 2 hrs then it was cooled down to RT, filtered and washed with MeOH. The solvent was evaporated and the residue was charged on a SCX cartridge washing with MeOH and eluting with 2 M NH₃ in MeOH. The fractions eluted with ammonia were combined and evaporated to dryness to give tert-butyl 1,4-dioxa-8,13-diazadispiro[4.0.46.45]tetradecane-13-carboxylate (p38, 1.33 g, y=96%) as a colourless oil.

MS (ES) (m/z): 299.2 [M+H]⁺.

### Preparation 39: tert-butyl 8-[bis(4-fluorophenyl)methyl]-1,4-dioxa-8,13-diazadispiro[4.0.4⁶.4⁵]tetradecane-13-carboxylate (P39)

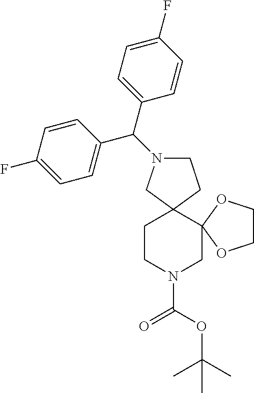 [SMILES: CC(C)(C)OC(=O)N1CCC2(CCN(C(c3ccc(F)cc3)c3ccc(F)cc3)C2)C2(C1)OCCO2]

Chlorobis(4-fluorophenyl)methane (0.15 mL, 0.804 mmol) was added to a stirred mixture of tert-butyl 1,4-dioxa-8,13-diazadispiro[4.0.46.45]tetradecane-13-carboxylate (p38, 200 mg, 0.67 mmol) and K₂CO₃ (345 mg, 1.675 mmol) in Acetonitrile (5 mL). The mixture was stirred overnight at reflux. The solution was filtered washing with EtOAc. The residue was purified by FC on silica gel (eluent: cHex/EtOAc from 95/5 to 80/20) to give tert-butyl 8-[bis(4-fluorophenyl)methyl]-1,4-dioxa-8,13-diazadispiro[4.0.46.45]tetradecane-13-carboxylate (p39, 65 mg, y=19%) as a white solid.

MS (ES) (m/z): 501.3 [M+H]⁺.

### Example 20: 8-[bis(4-fluorophenyl)methyl]-1,4-dioxa-8,13-diazadispiro[4.0.4⁶.4⁵]tetradecane (E20)

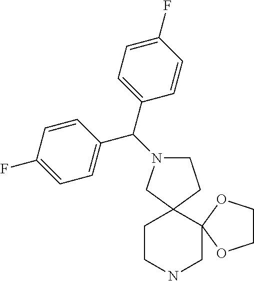 [SMILES: Fc1ccc(C(c2ccc(F)cc2)N2CCC3(CCNCC34OCCO4)C2)cc1]

HCl 2N (2 mL) was added to a solution of tert-butyl 8-[bis(4-fluorophenyl)methyl]-1,4-dioxa-8,13-diazadispiro[4.0.46.45]tetradecane-13-carboxylate (p39, 65 mg, 0.13 mmol) in THF (3 mL). The solution was stirred at RT overnight. The day after 4 drops of HCl 37% were added, the solution was warmed to reflux and it was stirred at that temperature for 1.5 h. The solution was cooled down to RT and concentrated; residue was charged on a SCX cartridge washing with MeOH and eluting with 2M NH₃ in MeOH. The fractions eluted with ammonia were combined and evaporated to dryness to give 8-[bis(4-fluorophenyl)methyl]-1,4-dioxa-8,13-diazadispiro[4.0.46.45]tetradecane (E20, 59 mg, 0.14 mmol) as a white solid.

MS (ES) (m/z): 401.2 [M+H]⁺.

¹H NMR (CHLOROFORM-d): δ ppm 7.40 (m, 4H), 6.98 (td, 4H), 4.17 (s, 1H), 3.92-4.08 (m, 4H), 2.76 (t, 2H) 2.51-2.70 (m, 3H), 2.35-2.49 (m, 2H), 2.28 (q, 1H), 2.00 (m, 1H), 1.82 (t, 2H), 1.60 (m, 1H)

### Preparation 40: ethyl 1-benzyl-4-(2-methoxyethyl)-3-oxopiperidine-4-carboxylate (P40)

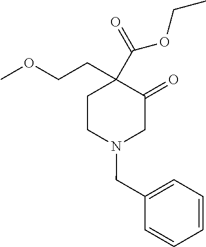 [SMILES: CCOC(=O)C1(CCOC)CCN(Cc2ccccc2)CC1=O]

To a suspension of 1-benzyl-3-piperidone-4-carboxylic acid benzyl ester hydrochloride (5 g, 16.79 mmol) in DMF (50 mL) at 0° C. was added t-BuOK (5.65 g, 50.37 mmol) and the mixture was warmed to RT and stirring was continued for 30 min. A solution of 1-Bromo-2-methoxyethane (3.155 mL, 33.58 mmol) in DMF (10 mL) was added followed by the addition of NaI (1.26 g, 8.4 mmol). The resulting mixture was heated at 80° C. for 2 hrs followed by 10 hrs at 50° C. After cooling to RT, the reaction mixture was diluted with Et₂O (50 mL×2); the organic phase was washed with water, brine, dried and evaporated. Crude material was purified by FC on silica gel (eluent: cHex to EtOAc 100%) affording ethyl 1-benzyl-4-(2-methoxyethyl)-3-oxopiperidine-4-carboxylate (p40, 1.1 g, y=20%) as yellow oil.

MS (ES) (m/z): 320.2 [M+H]⁺.

### Preparation 41: ethyl 1-benzyl-3-hydroxy-4-(2-methoxyethyl)piperidine-4-carboxylate (P41)

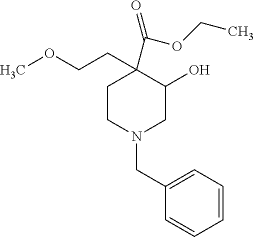 [SMILES: CCOC(=O)C1(CCOC)CCN(Cc2ccccc2)CC1O]

To a stirred solution of ethyl 1-benzyl-4-(2-methoxyethyl)-3-oxopiperidine-4-carboxylate (p40, 1.1 g, 3.44 mmol) in MeOH (10 mL), cooled with an ice bath, NaBH₄ (157 mg, 4.13 mmol) was added portionwise. The mixture was stirred at RT for 1 h. The reaction mixture was quenched with 1N NaOH and diluted with EtOAc. The organic layer was separated, washed with brine, dried and concentrated to give ethyl 1-benzyl-3-hydroxy-4-(2-methoxyethyl)piperidine-4-carboxylate (p41, 750 mg, y=67%) as mixture of diasteroisomers which was used as such.

MS (ES) (m/z): 322.2 [M+H]⁺.

### Preparation 42: bis(4-fluorophenyl)methanamine (P42)

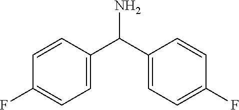 [SMILES: NC(c1ccc(F)cc1)c1ccc(F)cc1]

A mixture of bis(4-fluorophenyl)methanone (10 g, 45.83 mmol) and formamide (65 mL) was heated open to air at 175° C. After 18 hrs the dark solution was poured while hot into water and then DCM was added. The organic phase was washed with water, dried and concentrated under reduced pressure. This crude amidic intermediate was treated with 40% aq. NaOH (40 mL) and EtOH (180 mL), the resulting mixture was heated to reflux for 2 hrs before it was allowed to cool down to RT. The volume was reduced to 120 mL by rotary evaporator. The remaining mixture was extracted with DCM. The organic phase was washed with water, dried and concentrated under reduced pressure. The crude material was purified by SCX cartridge washing with MeOH and eluting with 2N NH₃/MeOH to give bis(4-fluorophenyl)methanamine (p42, 7.15 g, y=71%)

MS (ES) (m/z): 203.1 [M-NH₂]⁺.

### Preparation 43: 2-[bis(4-fluorophenyl)methyl]-6-hydroxy-2,8-diazaspiro[4.5]decan-1-one (P43)

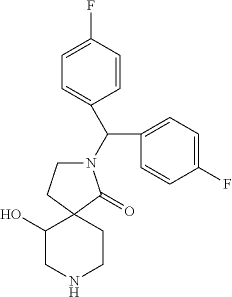 [SMILES: O=C1N(C(c2ccc(F)cc2)c2ccc(F)cc2)CCC12CCNCC2O]

Step a:

To a stirred solution of ethyl 1-benzyl-3-hydroxy-4-(2-methoxyethyl)piperidine-4-carboxylate (p41, 300 mg, 0.931 mmol) and bis(4-fluorophenyl)methanamine (p42, 405 mg, 1.86 mmol) in Toluene (20 mL) at RT, Et₂AlCl 1M solution in hexane (1.86 mL, 1.86 mmol) was added dropwise. Once the addition was complete, the mixture was stirred at reflux for 24 hrs. The reaction mixture was cooled down to RT and quenched with Na₂SO₄*10H₂O, filtered washing with EtOAc and evaporated. The crude material was purified by FC on silica gel (eluent: cHex to EtOAc) affording 8-benzyl-2-[bis(4-fluorophenyl)methyl]-6-hydroxy-2,8-diazaspiro[4.5]decan-1-one (73 mg) as yellow oil that was combined with a second batch of compound prepared with similar procedure to give a total amount of 100 mg.

Step b:

To a stirred solution of 8-benzyl-2-[bis(4-fluorophenyl)methyl]-6-hydroxy-2,8-diazaspiro[4.5]decan-1-one (from step a, 100 mg) in MeOH (10 mL) at RT, Pd/C (100 mg) was added and the mixture was stirred under H₂ atmosphere (1 atm) at RT for 16 hrs. The mixture was filtered through a pad of celite washing with MeOH and the solvent was removed under reduced pressure. Crude material was purified by FC on NH column (eluent: cHex to EtOAc/MeOH 85/15) affording 2-[bis(4-fluorophenyl)methyl]-6-hydroxy-2,8-diazaspiro[4.5]decan-1-one (p43, 52 mg) as colourless oil.

MS (ES) (m/z): 373.2 [M+H]⁺.

### Example 21: 2-[bis(4-fluorophenyl)methyl]-2,8-diazaspiro[4.5]decan-6-ol (E21)

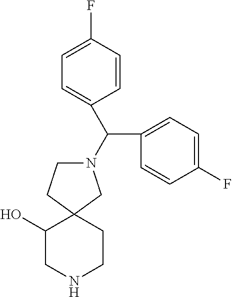 [SMILES: OC1CNCCC12CCN(C(c1ccc(F)cc1)c1ccc(F)cc1)C2]

To a stirred solution of 2-[bis(4-fluorophenyl)methyl]-6-hydroxy-2,8-diazaspiro[4.5]decan-1-one (p43, 52 mg, 0.139 mmol) in THF (5 mL) at RT 1M LiAlH₄ in THF (0.140 mL) was added dropwise and the mixture was stirred at reflux for 5 hrs. The mixture was cooled down to −10° C. and Na₂SO₄*10H₂O was added; after 1 h stirring at RT, the mixture was filtered using EtOAc. After solvent evaporation the residue was dissolved in MeOH (3 mL) and NaBH₄ (8 mg, 0.208 mmol) was added. The resulting mixture was stirred for 1 h. Reaction was quenched with water, MeOH was evaporated and the product was extracted with DCM (3 mL×3). The organic phase was dried and concentrated under reduced pressure to afford 2-[bis(4-fluorophenyl)methyl]-2,8-diazaspiro[4.5]decan-6-ol (E21, 47 mg, y=95%) as yellow foam.

MS (ES) (m/z): 359.17 [M+H]⁺.

¹H NMR (CHLOROFORM-d): δ ppm 1.44-1.78 (m, 3H), 1.85-1.99 (m, 2H), 2.13-2.30 (m, 1H), 2.47 (br. s., 1H), 2.53-2.64 (m, 1H), 2.69-2.84 (m, 2H), 2.99-3.13 (m, 2H), 3.44-3.49 (m, 1H), 4.09-4.15 (m, 1H), 4.42-5.72 (m, 1H), 6.98 (dt, 4H), 7.28-7.41 (m, 4H)

### Preparation 44: tert-butyl 1-oxo-2,7-diazaspiro[4.5]decane-7-carboxylate (P44)

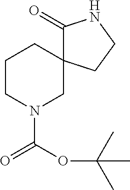 [SMILES: CC(C)(C)OC(=O)N1CCCC2(CCNC2=O)C1]

2,7-diazaspiro[4.5]decan-1-one hydrochloride (600 mg, 3.15 mmol), was dissolved in 30 mL of DCM. To this solution, TEA (1.98 mL, 14.18 mmol) and Boc₂O (895 mg, 4.10 mmol) were added and the reaction mixture was stirred at RT for 4 hrs. Water was added and the two layers were separated; the organic layer was washed with NH₄Cl, dried and evaporated to obtain tert-butyl 1-oxo-2,7-diazaspiro[4.5]decane-7-carboxylate (p44, 830 mg, y=quant.), colourless oil.

MS (ES) (m/z): 255.2 [M+H]⁺.

### Preparation 45: tert-butyl 2-[bis(4-fluorophenyl)methyl]-1-oxo-2,7-diazaspiro[4.5]decane-7-carboxylate P45

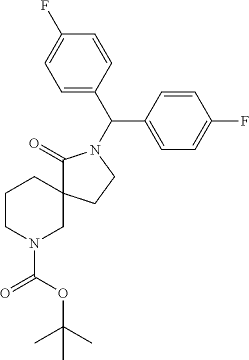 [SMILES: CC(C)(C)OC(=O)N1CCCC2(CCN(C(c3ccc(F)cc3)c3ccc(F)cc3)C2=O)C1]

To a solution of tert-butyl 1-oxo-2,7-diazaspiro[4.5]decane-7-carboxylate (p44, 150 mg, 0.589 mmol) in dry DMF (2 mL) was added NaH (60% dispersion in mineral oil, 28.27 mg, 0.7 mmol) followed by addition of 1-[chloro(4-fluorophenyl)methyl]-4-fluorobenzene (0.121 mL, 0.648 mmol). The mixture was heated at 100° C. overnight, then cooled down to RT and the solvent was removed under vacuum. The residue was dissolved in ethyl acetate and washed with water. Organic phase was dried and concentrated under reduced pressure. Crude material was purified by FC on silica gel (eluent: Cy to Cy/EA to 70/30) affording tert-butyl 2-[bis(4-fluorophenyl)methyl]-1-oxo-2,7-diazaspiro[4.5]decane-7-carboxylate (p45, 80 mg, y=30%) as white solid.

MS (ES) (m/z): 457.2 [M+H]⁺.

### Preparation 46: tert-butyl 2-[bis(4-fluorophenyl)methyl]-2,7-diazaspiro[4.5]decane-7-carboxylate (P46)

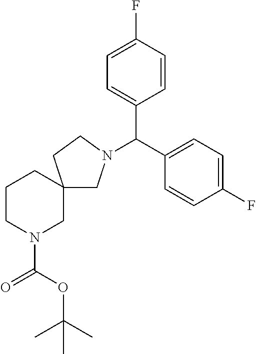 [SMILES: CC(C)(C)OC(=O)N1CCCC2(CCN(C(c3ccc(F)cc3)c3ccc(F)cc3)C2)C1]

BH₃ Me₂S complex 2M solution in THF (0.7 mL, 1.5 mmol) was added to an ice cooled solution of tert-butyl 2-[bis(4-fluorophenyl)methyl]-1-oxo-2,7-diazaspiro[4.5]decane-7-carboxylate (p45, 80 mg, 0.175 mmol) in THF (5 mL). The resulting solution was stirred at RT for 4 hrs, then further 10 eq of BH₃ Me₂S complex 2M solution in THF were added and the mixture stirred at RT for further 12 hrs. MeOH (3 mL) was added and the solution was stirred at RT for 20 hrs. The solvent was evaporated and the crude material was purified by FC on NH column (eluent: cHex to EtOAc) affording tert-butyl 2-[bis(4-fluorophenyl)methyl]-2,7-diazaspiro[4.5]decane-7-carboxylate (p46, 44 mg, y=57%) as colorless oil.

MS (ES) (m/z): 443.3 [M+H]⁺.

### Example 22: 2-[bis(4-fluorophenyl)methyl]-2,7-diazaspiro[4.5]decane (E22)

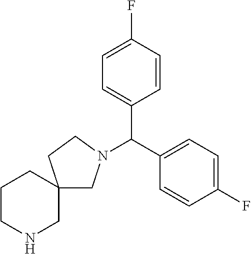 [SMILES: Fc1ccc(C(c2ccc(F)cc2)N2CCC3(CCCNC3)C2)cc1]

TFA (0.5 mL) was added to a solution of tert-butyl 2-[bis(4-fluorophenyl)methyl]-2,7-diazaspiro[4.5]decane-7-carboxylate (p46, 44 mg, 0.099 mmol) in DCM (3 mL). The solution was stirred for 1 h at RT, then the solvent was evaporated and the crude material was charged on a SCX cartridge washing with MeOH and eluting with 1M NH₃ in MeOH to afford 2-[bis(4-fluorophenyl)methyl]-2,7-diazaspiro[4.5]decane (E22, 29 mg, y=85%) as pale yellow oil.

MS (ES) (m/z): 343.3 [M+H]⁺.

¹H NMR (CHLOROFORM-d): δ ppm 7.38 (dd, 4H), 6.97 (td, 4H), 4.14 (s, 1H), 2.64-2.79 (m, 4H), 2.45-2.54 (m, 1H), 2.34-2.44 (m, 2H), 2.19 (d, 1H), 1.59-1.72 (m, 2H), 1.46-1.59 (m, 4H)

### Example 23: 2-[bis(4-fluorophenyl)methyl]-7-methyl-2,7-diazaspiro[4.5]decane (E23)

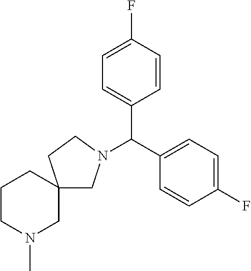 [SMILES: CN1CCCC2(CCN(C(c3ccc(F)cc3)c3ccc(F)cc3)C2)C1]

To a solution of 2-[bis(4-fluorophenyl)methyl]-2,7-diazaspiro[4.5]decane (E22, 22 mg, 0.064 mmol) in DMC (3 mL) formaldehyde 37% w/w in water (0.045 mL, 0.64 mmol) was added and the mixture was stirred at RT for 15 min. NaBH(OAc)₃ (55 mg, 0.257 mmol) was then added and the mixture was stirred at RT for 1 h. Reaction was then concentrated and charged on a SCX cartridge washing with MeOH and eluting with 1M NH₃ in MeOH to afford 2-[bis(4-fluorophenyl)methyl]-7-methyl-2,7-diazaspiro[4.5]decane (E23, 20 mg, y=87%) as colorless oil

MS (ES) (m/z): 357.2 [M+H]⁺.

¹H NMR (CHLOROFORM-d): δ ppm 7.47 (dd, 4H), 7.01 (t, 4H), 4.22 (s, 1H), 2.25-2.53 (m, 8H), 2.22 (s, 3H), 1.43-1.70 (m, 6H)

### Preparation 47: 7-[bis(4-fluorophenyl)methyl]-2,7-diazaspiro[4.5]decan-1-one (P47)

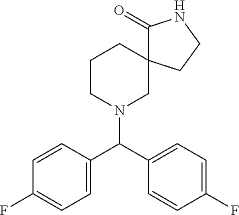 [SMILES: O=C1NCCC12CCCN(C(c1ccc(F)cc1)c1ccc(F)cc1)C2]

Chlorobis(4-fluorophenyl)methane (0.174 mL, 0.934 mmol) was added to a stirred mixture of 2,7-diazaspiro[4.5]decan-1-one hydrochloride salt (0.15 g, 0.78 mmol) and K₂CO₃ (0.27 g, 1.95 mmol) in Acetonitrile (3 mL). The mixture was stirred for 3 hrs at reflux. The solution was diluted with EtOAc and water. The organic phase was dried and evaporated. The residue was purified by FC on silica gel (eluent: cHex to EtOAc) to afford 7-[bis(4-fluorophenyl)methyl]-2,7-diazaspiro[4.5]decan-1-one (p47, 165 mg, y=59%) as white foam.

MS (ES) (m/z): 357.2 [M+H]⁺.

### Example 24: 7-[bis(4-fluorophenyl)methyl]-2,7-diazaspiro[4.5]decane (E24)

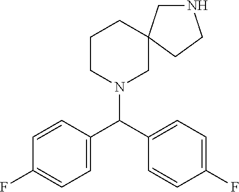 [SMILES: Fc1ccc(C(c2ccc(F)cc2)N2CCCC3(CCNC3)C2)cc1]

BH₃ Me₂S complex 2M solution in THF (0.56 mL, 1.12 mmol) was added to an ice cooled solution of 7-[bis(4-fluorophenyl)methyl]-2,7-diazaspiro[4.5]decan-1-one (p47, 50 mg, 0.14 mmol) in THF (4 mL). The resulting solution was stirred at RT for 4 hrs, then further BH₃ Me₂S complex 2M solution in THF (1 mL) was added and the mixture was stirred overnight at RT. MeOH (2 mL) was added and the solution was stirred at RT overnight. The solvent was evaporated and the crude material was purified by FC on silica gel (eluent: Cy to EtOAc) to give 7-[bis(4-fluorophenyl)methyl]-2,7-diazaspiro[4.5]decane (E24, 2.9 mg, y=6%).

MS (ES) (m/z): 343.2 [M+H]⁺.

¹H NMR (CHLOROFORM-d): δ ppm 7.33 (dd, 4H), 7.02 (td, 4H), 4.27 (s, 1H), 3.84 (br. s., 1H), 3.45 (br. s., 1H), 3.20 (m, 1H), 2.82-2.96 (m, 1H), 2.56-2.68 (m, 1H), 2.45 (br. s., 1H), 2.17 (br. s., 2H), 2.06 (br. s., 1H), 1.81 (dd, 3H), 1.42-1.52 (m, 1H), 1.24-1.39 (m, 1H)

### Preparation 48: 1-tert-butyl 4-ethyl 4-(2-cyanoethyl)piperidine-1,4-dicarboxylate (P48)

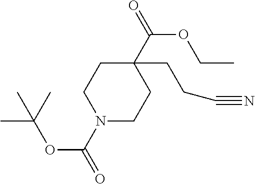 [SMILES: CCOC(=O)C1(CCC#N)CCN(C(=O)OC(C)(C)C)CC1]

To a stirred solution of 1-tert-butyl 4-ethyl piperidine-1,4-dicarboxylate (4.39 g, 17.07 mmol) in THF (70 mL), at −78° C. and under a nitrogen atmosphere, LDA (1.5 M solution in Hexane, 23 mL, 34.14 mmol) was added dropwise and the resulting dark orange solution was stirred 0.5 h at −78° C. 3-bromopropanenitrile (2 mL, 23.90 mmol) was added dropwise, and then the reaction mixture was allowed to reach −30° C. over 4.5 hrs. Saturated NH₄Cl solution and EA were added to the reaction mixture. The organic phase was washed with water and brine, and then dried. After removal of the solvent under reduced pressure the crude product was purified by FC on silica gel (eluent: Cy to Cy/EA 75/25) to give 1-tert-butyl 4-ethyl 4-(2-cyanoethyl)piperidine-1,4-dicarboxylate (p48, 2.12 g, y=40%) as pale yellow oil.

MS (ES) (m/z): 311.3 [M+H]⁺.

### Preparation 49: tert-butyl 1-oxo-2,9-diazaspiro[5.5]undecane-9-carboxylate (P49)

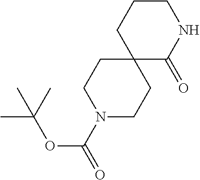 [SMILES: CC(C)(C)OC(=O)N1CCC2(CCCNC2=O)CC1]

A mixture of 1-tert-butyl 4-ethyl 4-(2-cyanoethyl)piperidine-1,4-dicarboxylate (p48, 1.59 g, 5.12 mmol) in Acetic acid (30 mL) and PtO₂ (0.23 g, 1.02 mmol) was hydrogenated at 5.5 atm in autoclave (Parr) under vigorous mechanical stirring, overnight. The mixture was filtered on celite and the solvent removed under reduce pressure. This material was dissolved in DCM and the mixture was washed with saturated NaHCO₃, dried and the solvent removed under reduced pressure to give tert-butyl 1-oxo-2,9-diazaspiro[5.5]undecane-9-carboxylate (p49, 1.03 g, y=75%) as white solid.

MS (ES) (m/z): 269.2 [M+H]⁺.

### Preparation 50: tert-butyl 1-oxo-2,9-diazaspiro[5.5]undecane-9-carboxylate (P50)

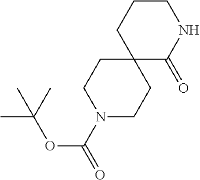 [SMILES: CC(C)(C)OC(=O)N1CCC2(CCCNC2=O)CC1]

Step a:

a mixture of 1-tert-butyl 4-ethyl 4-(2-cyanoethyl)piperidine-1,4-dicarboxylate (p48, 3.91 g, 12.60 mmol) in AcOH (70 mL) and PtO₂ (0.57 g, 2.52 mmol) was hydrogenated at 5.5 atm in autoclave (Parr) under vigorous mechanical stirring, overnight at RT. The mixture was filtered on celite and the solvent removed under reduce pressure. The residue was dissolved in DCM and the solution washed twice with saturated NaHCO₃, dried and concentrated under reduced pressure.

Step b:

To a stirred solution of 4-(3-aminopropyl)-1-[(tert-butoxy)carbonyl]piperidine-4-carboxylic acid (from step a, 3.61 g, 11.49 mmol) in MeOH/THF (30/5 mL), a solution of LiOH.H₂O (1.45 g, 34.47 mmol) in water (10 mL) was added and the reaction mixture was stirred overnight at 50° C. (external temperature). The reaction mixture was allowed to reach RT then it was concentrated under reduced pressure in order to remove the organic solvents and extracted twice with DCM. The organic phase was washed with saturated NaHCO₃, dried and the solvent removed under vacuum. The crude material was purified by FC on silica gel (eluent: DCM to DCM/MeOH 98/2) to give tert-butyl 1-oxo-2,9-diazaspiro[5.5]undecane-9-carboxylate (p50, 1.49 g, y=44%) as white solid.

MS (ES) (m/z): 269.2 [M+H]⁺.

### Preparation 51: tert-butyl 2-[bis(4-fluorophenyl)methyl]-1-oxo-2,9-diazaspiro[5.5]undecane-9-carboxylate (P51)

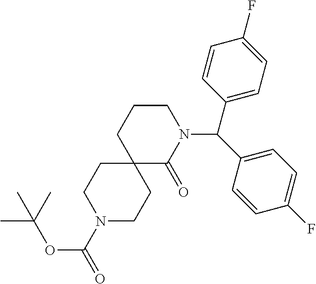 [SMILES: CC(C)(C)OC(=O)N1CCC2(CCCN(C(c3ccc(F)cc3)c3ccc(F)cc3)C2=O)CC1]

To a solution of tert-butyl 1-oxo-2,9-diazaspiro[5.5]undecane-9-carboxylate (p50, 165 mg, 0.61 mmol) in DMF (2 mL), at RT, NaH 60% dispersion in mineral oil (29 mg, 0.73 mmol) was added portion-wise followed, after 10 min, by 1-[chloro(4-fluorophenyl)methyl]-4-fluorobenzene (0.13 mL, 0.68 mmol). The mixture was heated to 100° C. (external temperature) and stirred overnight. The solvent was removed under vacuum and the crude material was purified by FC on silica gel (eluent: Cy to Cy/EA 70/30) to give tert-butyl 2-[bis(4-fluorophenyl)methyl]-1-oxo-2,9-diazaspiro[5.5]undecane-9-carboxylate (p51, 85 mg, y=30%).

MS (ES) (m/z): 471.3 [M+H]⁺.

### Preparation 52: 2-[bis(4-fluorophenyl)methyl]-2,9-diazaspiro[5.5]undecan-1-one (P52)

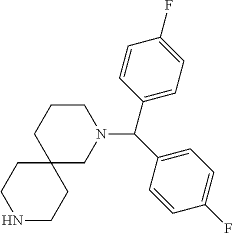 [SMILES: Fc1ccc(C(c2ccc(F)cc2)N2CCCC3(CCNCC3)C2)cc1]

To a solution of tert-butyl 2-[bis(4-fluorophenyl)methyl]-1-oxo-2,9-diazaspiro[5.5]undecane-9-carboxylate (p51, 85 mg, 0.18 mmol) in DCM (1.5 mL), at RT, TFA (0.1 mL) was added. After 2 hrs, the reaction mixture was concentrated under vacuum. The residue was dissolved in MeOH and the solution was charged on a SCX cartridge washing with MeOH and eluting with 2N NH₃ in MeOH to give 2-[bis(4-fluorophenyl)methyl]-2,9-diazaspiro[5.5]undecan-1-one (p52, 65 mg, y=97%) as white foam that was used as such in next step.

MS (ES) (m/z): 371.2 [M+H]⁺.

### Example 25: 2-[bis(4-fluorophenyl)methyl]-2,9-diazaspiro[5.5]undecane (E25)

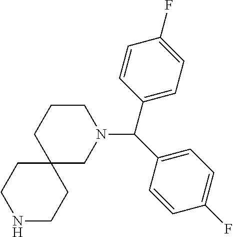 [SMILES: Fc1ccc(C(c2ccc(F)cc2)N2CCCC3(CCNCC3)C2)cc1]

LiAlH₄ (1M/THF) (0.24 mL, 0.24 mmol) was added to a solution of 2-[bis(4-fluorophenyl)methyl]-2,9-diazaspiro[5.5]undecan-1-one (p52, 60 mg, 0.16 mmol) in THF (1.5 mL) at 0° C.; the ice-bath was removed and the reaction mixture was brought to reflux. Additional LiAlH₄ (1M/THF) (0.1 mL) was added and the reaction mixture was refluxed for further 1 h. The stirred reaction mixture was cooled down to −10° C. and Na₂SO₄*10H₂O was carefully added portion-wise up to fizz end. The mixture was left stirring at RT for 30 min, then it was filtered, the solid was washed with DCM and the solvent concentrated under reduced pressure. The crude product was purified by FC on NH column (eluent: DCM to DCM/MeOH 98/2) to give 2-[bis(4-fluorophenyl)methyl]-2,9-diazaspiro[5.5]undecane (E25, 13 mg, y=23%).

MS (ES) (m/z): 357.2 [M+H]⁺.

¹H NMR (CHLOROFORM-d): δ ppm 7.30-7.36 (m, 4H), 6.98 (t, 4H), 4.19 (s, 1H), 3.20 (br. s., 3H), 2.79-2.91 (m, 2H), 2.63-2.74 (m, 2H), 2.20-2.39 (m, 2H), 2.14 (br. s., 2H), 1.89 (s, 1H), 1.58 (d, 2H), 1.38 (br. s., 2H)

### Preparation 53: 2,9-diazaspiro[5.5]undecan-1-one hydrochloride(P53)

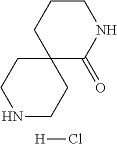

To a solution of tert-butyl 1-oxo-2,9-diazaspiro[5.5]undecane-9-carboxylate (p50, 500 mg, 1.86 mmol) in dioxane (2 mL), at RT, HCl (4N in dioxane) (2.3 mL) was added. The reaction mixture was stirred at RT for 6 hrs, then concentrated under vacuum to give 2,9-diazaspiro[5.5]undecan-1-one hydrochloride (p53, 380 mg, y=73%).

MS (ES) (m/z): 169.1 [M+H]⁺.

### Preparation 54: 9-[bis(4-fluorophenyl)methyl]-2,9-diazaspiro[5.5]undecan-1-one (P54)

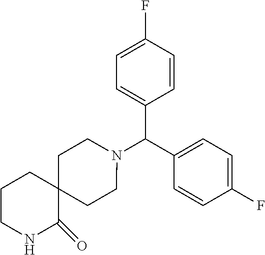 [SMILES: O=C1NCCCC12CCN(C(c1ccc(F)cc1)c1ccc(F)cc1)CC2]

Chlorobis(4-fluorophenyl)methane (0.064 mL, 0.342 mmol) was added to a stirred mixture of 2,9-diazaspiro[5.5]undecan-1-one hydrochloride (p53, 50 mg, 0.244 mmol) and K₂CO₃ (135 mg, 0.976 mmol) in Acetonitrile (3 mL). The mixture was stirred overnight at reflux. The reaction mixture was cooled down to RT, filtered washing the solid with EtOAc then solvent evaporated. The residue was loaded on a SCX cartridge washing with MeOH and eluting with NH₃ 1M in MeOH. Solvent was eliminated under reduced pressure affording 9-[bis(4-fluorophenyl)methyl]-2,9-diazaspiro[5.5]undecan-1-one (p54, 79 mg, y=87%).

MS (ES) (m/z): 371.2 [M+H]⁺.

### Example 26: 9-[bis(4-fluorophenyl)methyl]-2,9-diazaspiro[5.5]undecane (E26)

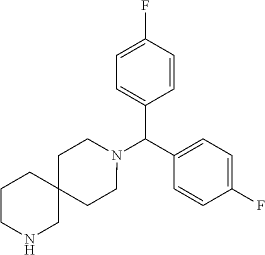 [SMILES: Fc1ccc(C(c2ccc(F)cc2)N2CCC3(CCCNC3)CC2)cc1]

LiAlH₄ 1M in THF (0.243 mL, 0.243 mmol) was added to solution of 9-[bis(4-fluorophenyl)methyl]-2,9-diazaspiro[5.5]undecan-1-one (p54, 45 mg, 0.121 mmol) in THF (3 mL) at 0° C. then the mixture was refluxed for 30 min, cooled down to −20° C. and quenched with Na₂SO₄*10H₂O. The mixture was left stirring at RT for 10 min, then it was filtered washing with AcOEt and solvent concentrated under reduced pressure affording 9-[bis(4-fluorophenyl)methyl]-2,9-diazaspiro[5.5]undecane (E26, 40.6 mg, y=94%) as white foam.

MS (ES) (m/z): 357.2 [M+H]⁺.

¹H NMR (CHLOROFORM-d): δ ppm 7.35 (dd, 4H), 6.98 (t, 4H), 4.26 (s, 1H), 2.77 (t, 1H), 2.62 (s, 1H), 2.30 (br. s., 4H), 1.42-1.67 (m, 11H)

### Preparation 55: 1-tert-butyl 4-ethyl 4-(prop-2-en-1-yl)piperidine-1,4-dicarboxylate (P55)

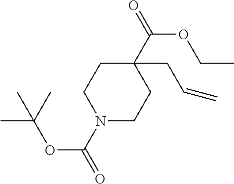 [SMILES: C=CCC1(C(=O)OCC)CCN(C(=O)OC(C)(C)C)CC1]

To a solution of 1-tert-butyl 4-ethyl piperidine-1,4-dicarboxylate (2.0 g, 7.77 mmol) in THF (12 mL), at −78° C. and under a nitrogen atmosphere, LiHMDS 1 m solution in THF (10.1 mL. 10.1 mmol) was added dropwise and the reaction mixture was stirred at this temperature for 30 min. 3-bromoprop-1-ene (1.0 mL, 11.66 mmol) was added dropwise, the reaction mixture was allowed to reach RT and stirred at that temperature overnight. The reaction mixture was treated with concentrated NH₄Cl and extracted with EA, the organic phase was washed with water, brine and the solvent removed under reduced pressure. The crude product was purified by FC on silica gel (eluent: Cy to Cy/EA 95/5) to give 1-tert-butyl 4-ethyl 4-(prop-2-en-1-yl)piperidine-1,4-dicarboxylate (p55, 2.05 g, y=89%) as a colourless oil.

¹H NMR (CHLOROFORM-d): δ ppm 5.69 (ddt, 1H), 5.00-5.13 (m, 2H), 4.18 (q, 2H), 3.88 (br. s., 2H), 2.91 (br. s., 2H), 2.28 (d, 2H), 2.08 (s, 2H), 2.11 (s, 2H), 1.46 (s, 9H), 1.23-1.32 (m, 3H)

### Preparation 56: 1-tert-butyl 4-ethyl 4-(2-oxoethyl)piperidine-1,4-dicarboxylate (P56)

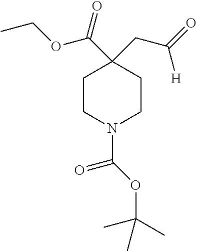 [SMILES: CCOC(=O)C1(CC=O)CCN(C(=O)OC(C)(C)C)CC1]

1-tert-butyl 4-ethyl 4-(prop-2-en-1-yl)piperidine-1,4-dicarboxylate (p55, 1.8 g, 6.06 mmol) was dissolved in THF/water (30/30 mL) and a solution of OsO₄ 4% in water (4.5 mL, 0.5 mmol) was added. After 5 min NaIO₄ (3.2 g, 15.15 mmol) was added and the mixture was stirred at RT for 3 hrs. EtOAc was added and the organic phase was washed with NaHCO₃, the aqueous phase was extracted with EtOAC and the combined organic layers concentrated under reduced pressure affording 1-tert-butyl 4-ethyl 4-(2-oxoethyl)piperidine-1,4-dicarboxylate (p56, 1.98 g, y=quant.) as brown oil.

¹H NMR (CHLOROFORM-d): δ ppm 9.76 (s, 1H), 4.15 (q, 2H), 3.70 (br. s., 2H), 3.24 (t, 2H), 2.71 (s, 2H), 2.15 (dt, 2H), 1.50-1.59 (m, 2H), 1.48 (s, 9H), 1.29 (t, 3H)

### Preparation 57: tert-butyl 2-benzyl-1-oxo-2,8-diazaspiro[4.5]decane-8-carboxylate (P57)

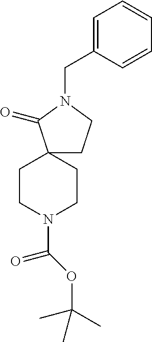 [SMILES: CC(C)(C)OC(=O)N1CCC2(CC1)CCN(Cc1ccccc1)C2=O]

To a solution of 1-tert-butyl 4-ethyl 4-(2-oxoethyl)piperidine-1,4-dicarboxylate (p56, 1.7 g, 5.68 mmol) and Benzylamine (0.81 mL, 7.39 mmol) in THF (30 mL), NaBH(OAc)₃ was added (2.4 g, 11.37 mmol) and the mixture was stirred at RT overnight. EtOAc was added and the organic phase was washed with NaHCO₃ solution. The aqueous phase was extracted twice with EtOAC. The combined organic phases were concentrated to dryness. The crude material was purified by FC on silica gel (eluent: Cy/EtOAc 50/50 to Cy/EtOAc 30/70) giving tert-butyl 2-benzyl-1-oxo-2,8-diazaspiro[4.5]decane-8-carboxylate (p57, 1.27 g, y=65%)

¹H NMR (CHLOROFORM-d): δ ppm 7.30-7.40 (m, 3H), 7.24 (d, 2H), 4.48 (s, 2H), 4.04 (br. s., 2H), 3.21 (t, 2H), 3.01 (t, 2H), 1.87-2.01 (m, 4H), 1.47-1.55 (m, 9H), 1.37-1.47 (m, 2H)

### Preparation 58: tert-butyl 2,8-diazaspiro[4.5]decane-8-carboxylate (P58)

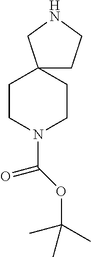 [SMILES: CC(C)(C)OC(=O)N1CCC2(CCNC2)CC1]

Step a

A solution of LiAlH₄ 2M in THF (2.7 mL, 2.7 mmol) was added dropwise to a solution of 1-tert-butyl 4-ethyl 4-(2-oxoethyl)piperidine-1,4-dicarboxylate (p57, 1.2 g, 3.48 mmol) in THF (20 mL) cooled to −20° C. The reaction mixture was stirred at RT for 2 hrs and then quenched by addition of Na₂SO₄.*10H₂O. The suspension was filtered on a pad of celite and concentrated to dryness. The crude material was purified by FC on silica gel (eluent: DCM/MeOH, from 0 to 10%), giving tert-butyl 2,8-diazaspiro[4.5]decane-8-carboxylate (0.986 g)

Step b

To a solution of tert-butyl 2,8-diazaspiro[4.5]decane-8-carboxylate (from step a, 0.78 g, 2.36 mmol) in MeOH (25 mL), ammonium formate (0.9 g, 14.3 mmol) and 10% Pd/C (0.3 g) were added at RT and the mixture was stirred at reflux for 1 h. The mixture was cooled down to RT, filtered through a pad of celite washing with MeOH and the solution concentrated to dryness. The crude material was loaded on a SCX cartridge washing with MeOH and eluting with NH₃ 2M in MeOH, affording tert-butyl 2,8-diazaspiro[4.5]decane-8-carboxylate (p58, 0.56 g, y=67%).

¹H NMR (CHLOROFORM-d): δ ppm 3.33-3.50 (m, 4H), 3.21 (t, 2H), 2.94 (s, 2H), 1.80 (t, 2H), 1.58 (t, 4H), 1.49 (s, 9H)

### Example 27: 2-[bis(4-fluorophenyl)methyl]-2,8-diazaspiro[4.5]decane (E27)

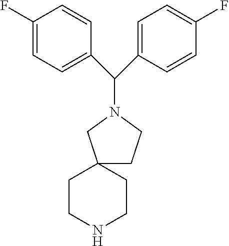 [SMILES: Fc1ccc(C(c2ccc(F)cc2)N2CCC3(CCNCC3)C2)cc1]

Step a

tert-butyl 2,8-diazaspiro[4.5]decane-8-carboxylate (p58, 0.1 g, 0.416 mmol) and 1-(chloro-(4-fluorophenyl)methyl)-4-fluorobenzene (0.109 mL, 0.457 mmol) were dissolved in Acetonitrile and K₂CO₃ (115 mg) was added. The mixture was heated to 80° C. overnight. DCM and water were added and the two phases separated. The aqueous one was extracted with DCM. The combined organic phases were concentrated to dryness. The residue was purified by FC on silica gel (eluent: DCM to DCM/MeOH 90/10) affording tert-butyl 2-[bis(4-fluorophenyl)methyl]-2,8-diazaspiro[4.5]decane-8-carboxylate (70 mg)

Step b

tert-butyl 2-[bis(4-fluorophenyl)methyl]-2,8-diazaspiro[4.5]decane-8-carboxylate (from step a, 70 mg, 0.158 mmol) was dissolved in DCM (2 mL) and TFA (0.12 mL) was added. The mixture was stirred at RT for 1 h and then concentrated to dryness. The crude was dissolved in MeOH and loaded on a SCX cartridge, washing with MeOH and eluting with NH₃ 2M in MeOH. The compound recovered (53 mg) was purified by prep HPLC (acidic conditions)

LC/MS Conditions:

Columns: XSelect CSH Prep. C18 5 μm OBD 30×100 mm at RT

Injection loop 1 mL

Solvents: A=H2O+0.1% HCOOH

B=Acetonitrile

Gradient:

| Time (min) | Flow Rate (mL/min) | % A | % B | Curve |
|---|---|---|---|---|
| initial | 43.0 | 97.0 | 3.0 | — |
| 10.0 | 43.0 | 50.0 | 50.0 | 6 |
| 10.5 | 43.0 | 0.0 | 100.0 | 6 |
| 14.5 | 43.0 | 0.0 | 100.0 | 6 |
| 15.0 | 43.0 | 97.0 | 3.0 | 6 |

The curve parameter followed Waters definition (6=linear, 11=step).

Acquisition stop time: 16.0 min

UV Conditions:

UV detection range: 210 nm to 350 nm

Acquisition rate: 1.0 spectra/s

MS Conditions:

Ionisation mode: Positive Electrospray (E⁺)

Scan Range: ES⁺100 to 900 AMU

Product recovered was charged on a SCX cartridge washing with MeOH and eluting with NH₃ 2M in MeOH, affording 2-[bis(4-fluorophenyl)methyl]-2,8-diazaspiro[4.5]decane (E27, 10.6 mg, y=7%)

MS (ES) (m/z): 343.25 [M+H]⁺.

¹H NMR (CHLOROFORM-d): δ ppm 7.33-7.44 (m, 4H), 6.98 (t, 4H), 4.15 (s, 1H), 2.71-2.87 (m, 4H), 2.46 (t, 2H), 2.26-2.34 (m, 2H), 1.62-1.65 (m, 2H), 1.58 (d, 4H)

### Example 28: 2-[bis(4-chlorophenyl)methyl]-2,8-diazaspiro[4.5]decane (E28)

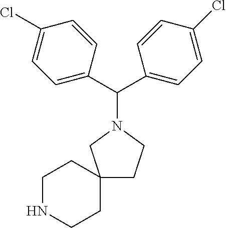 [SMILES: Clc1ccc(C(c2ccc(Cl)cc2)N2CCC3(CCNCC3)C2)cc1]

Step a

1-chloro-4-[chloro(4-chlorophenyl)methyl]benzene (217 mg, 0.799 mmol) was added to a stirred mixture of tert-butyl 2,8-diazaspiro[4.5]decane-8-carboxylate (p58, 160 mg, 0.666 mmol) and K₂CO₃ (230 mg, 1.665 mmol) in Acetonitrile (6 mL). The mixture was stirred for 18 hrs at reflux. The solution was filtered using EtOAc and evaporated. The crude material was purified by FC on silica gel (eluent: EtOAc to EtOAc/MeOH 90/10) affording tert-butyl 2-[bis(4-chlorophenyl)methyl]-2,8-diazaspiro[4.5]decane-8-carboxylate (150 mg) as colourless oil.

Step b

TFA (1 mL) was added to a solution of tert-butyl 2-[bis(4-chlorophenyl)methyl]-2,8-diazaspiro[4.5]decane-8-carboxylate (from step a, 150 mg, 0.315 mmol) in 5 mL of DCM. The mixture was stirred for 30 min, and then the solvent was removed under reduced pressure. The residue was loaded on SCX cartridge washing with MeOH and eluting with 2M NH₃ in MeOH to afford 2-[bis(4-chlorophenyl)methyl]-2,8-diazaspiro[4.5]decane (E28, 110 mg, y=44%) as colourless oil.

MS (ES) (m/z): 375.16 [M+H]⁺.

¹H NMR (CHLOROFORM-d): δ ppm 7.36 (d, 4H), 7.26 (d, 4H), 4.14 (s, 1H), 2.71-2.86 (m, 4H), 2.47 (t, 2H), 2.30 (s, 2H), 1.61-1.66 (m, 2H), 1.56 (br. s., 4H)

### Preparation 59: 5-benzyl-1-oxa-5-azaspiro[2.4]heptanes (P59)

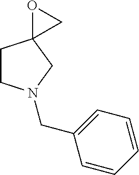 [SMILES: c1ccc(CN2CCC3(CO3)C2)cc1]

To an ice-cooled mixture of NaH (60%, 0.59 g, 14.83 mmol) and Trimethylsulfoxonium iodide (2.76 g, 12.55 mmol) was added DMSO (10 mL) keeping the mixture at 10° C. After stirring for 10 min at 10° C., the mixture was allowed to reach RT and left stirring at that temperature for 1 h. A solution of 1-benzylpyrrolidin-3-one (2 g, 11.41 mmol) in DMSO (10 mL) was added via syringe over 10 min. The resulting reaction mixture was stirred for 1.5 h at RT, diluted with Et₂O and quenched by the addition of saturated aqueous NH₄Cl. Phases were separated and aqueous one was backextracted with Et₂O. Combined organics were dried, filtered and concentrated under reduced pressure to give 5-benzyl-1-oxa-5-azaspiro[2.4]heptanes (p59, 2.10 g, crude material) that was used as such.

MS (ES) (m/z): 190.1 [M+H]⁺.

### Preparation 60: 3-(aminomethyl)-1-benzylpyrrolidin-3-ol (P60)

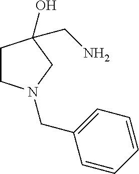 [SMILES: NCC1(O)CCN(Cc2ccccc2)C1]

To a stirred solution of 5-benzyl-1-oxa-5-azaspiro[2.4]heptanes (p59, 2.10 g, 11.1 mmol) in MeOH (12 mL), at 0° C., 28% aq. NH₄OH (25 mL), was added portionwise. After 5 min the ice-bath was removed and the resulting reaction mixture was stirred at RT overnight.

The reaction mixture was concentrated under reduced pressure, the residue was taken up with DCM and 1N NaOH, the organic phase was washed with water and brine, and then dried and concentrated under vacuum. The crude material was purified by FC on NH silica (eluent: DCM to DCM/MeOH 95/5) to give 3-(aminomethyl)-1-benzylpyrrolidin-3-ol (p60, 0.94 g, y=41%) as brown oil.

MS (ES) (m/z): 207.2 [M+H]⁺.

### Preparation 61: N-[(1-benzyl-3-hydroxypyrrolidin-3-yl)methyl]-2-chloroacetamide (P61)

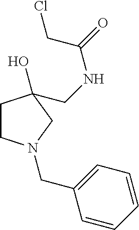 [SMILES: O=C(CCl)NCC1(O)CCN(Cc2ccccc2)C1]

To a stirred solution of 3-(aminomethyl)-1-benzylpyrrolidin-3-ol (p60, 0.84 g, 4.07 mmol) in DCM (9 mL), at 0° C. and under a nitrogen atmosphere, TEA (1.0 mL) was added followed by chloroacetylchloride (0.26 mL, 3.26 mmol) in DCM (2 mL) dropwise over 1 h.

The reaction mixture was allowed to reach RT and stirred at that temperature for 4 hrs.

The reaction mixture was diluted with DCM and saturated NH₄Cl, the organic phase was washed with brine, dried and concentrated under reduced pressure. The crude material was purified by FC on silica gel (eluent: DCM to DCM/MeOH 95/5) to give N-[(1-benzyl-3-hydroxypyrrolidin-3-yl)methyl]-2-chloroacetamide (p61, 0.33 g, y=28.7%)

MS (ES) (m/z): 283.2 [M+H]⁺.

### Preparation 62: 2-benzyl-6-oxa-2,9-diazaspiro[4.5]decan-8-one (P62)

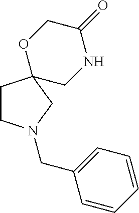 [SMILES: O=C1COC2(CCN(Cc3ccccc3)C2)CN1]

To a stirred solution of N-[(1-benzyl-3-hydroxypyrrolidin-3-yl)methyl]-2-chloroacetamide (p61, 220 mg, 0.778 mmol) in THF (35 mL), at 0° C. and under a nitrogen atmosphere, NaH 60% dispersion in mineral oil (62.25 mg, 1.55 mmol) was added portionwise and then the ice-bath was removed. After 2 hrs at RT, the reaction mixture was concentrated under vacuum. The residue was diluted with DCM and water, and then neutralized with 1N HCl. The organic layer was dried, filtered and concentrated under reduced pressure to obtain 2-benzyl-6-oxa-2,9-diazaspiro[4.5]decan-8-one (p62, 158 mg, crude material) as colourless oil.

MS (ES) (m/z): 247.2 [M+H]⁺.

### Preparation 63: 6-oxa-2,9-diazaspiro[4.5]decan-8-one (P63)

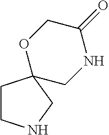 [SMILES: O=C1COC2(CCNC2)CN1]

To a solution of 2-benzyl-6-oxa-2,9-diazaspiro[4.5]decan-8-one (p62, 259 mg, 1.05 mmol) in MeOH (6 mL) ammonium formate (398 mg, 6.31 mmol) and 10% Pd/C (127 mg) were added at RT then the mixture was stirred under reflux for 1 h. The mixture was then cooled down to RT and filtered through a pad of celite washing with MeOH. Solvent was eliminated under reduced pressure affording 6-oxa-2,9-diazaspiro[4.5]decan-8-one (p63, 158 mg, y=96%) as colourless oil.

MS (ES) (m/z): 157.1 [M+H]⁺.

### Preparation 64: 2-[bis(4-fluorophenyl)methyl]-6-oxa-2,9-diazaspiro[4.5]decan-8-one (P64)

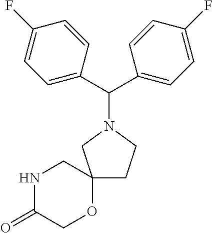 [SMILES: O=C1COC2(CCN(C(c3ccc(F)cc3)c3ccc(F)cc3)C2)CN1]

Chlorobis(4-fluorophenyl)methane (0.103 mL, 0.55 mmol) was added to a stirred mixture of 6-oxa-2,9-diazaspiro[4.5]decan-8-one (p63, 72 mg, 0.46 mmol) and K₂CO₃ (159 mg, 1.15 mmol) in Acetonitrile (3 mL). The mixture was stirred for 1 h at reflux. The solution was filtered washing with EtOAc and evaporated. The crude material was purified by FC on silica gel (eluent: DCM to DCM/MeOH 95/5). 2-[bis(4-fluorophenyl)methyl]-6-oxa-2,9-diazaspiro[4.5]decan-8-one (p64, 65 mg, y=40%) was obtained as pale yellow oil.

MS (ES) (m/z): 359.2 [M+H]⁺.

### Example 29: 2-[bis(4-fluorophenyl)methyl]-6-oxa-2,9-diazaspiro[4.5]decane (E29)

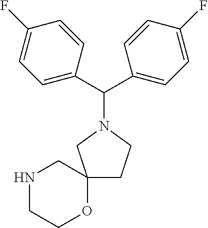 [SMILES: Fc1ccc(C(c2ccc(F)cc2)N2CCC3(CNCCO3)C2)cc1]

LiAlH₄ 2M in THF (0.135 mL, 0.272 mmol) was added to a solution of 2-[bis(4-fluorophenyl)methyl]-6-oxa-2,9-diazaspiro[4.5]decan-8-one (p64, 65 mg, 0.181 mmol) in THF (5.5 mL) at reflux for 1 h. The reaction was cooled down to −10° C., quenched with Na₂SO₄*10H₂O and the solvent was evaporated. The crude material was purified by FC on NH column (eluent: Cy to Cy/EtOAc 50/50). 2-[bis(4-fluorophenyl)methyl]-6-oxa-2,9-diazaspiro[4.5]decane (E29, 44 mg, y=71%) was obtained as colourless oil.

MS (ES) (m/z): 345.21 [M+H]⁺.

¹H NMR (METHANOL-d₄): δ ppm 7.48 (dd, 4H), 6.94-7.10 (m, 4H), 4.27 (s, 1H), 3.50-3.71 (m, 2H), 2.64-2.81 (m, 4H), 2.50-2.63 (m, 3H), 2.38-2.50 (m, 1H), 1.85-1.97 (m, 2H)

### Preparation 65: 2-[bis(4-chlorophenyl)methyl]-6-oxa-2,9-diazaspiro[4.5]decan-8-one (P65)

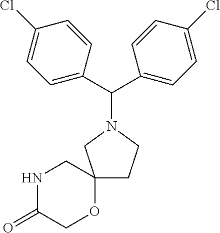 [SMILES: O=C1COC2(CCN(C(c3ccc(Cl)cc3)c3ccc(Cl)cc3)C2)CN1]

1-chloro-4-[chloro(4-chlorophenyl)methyl] benzene (177 mg, 0.653 mmol) was added to a stirred mixture of 6-oxa-2,9-diazaspiro[4.5]decan-8-one (p63, 85 mg, 0.544 mmol) and K₂CO₃ (188 mg, 1.36 mmol) in Acetonitrile (4 mL). The mixture was stirred for 6 hrs at reflux. The solution was filtered washing with EtOAc and then solvent was evaporated. The crude material was purified by FC on silica gel (eluent: DCM to DCM/MeOH 95/5). 2-[bis(4-chlorophenyl)methyl]-6-oxa-2,9-diazaspiro[4.5]decan-8-one (p65, 75 mg, y=35%) was obtained as pale yellow oil.

MS (ES) (m/z): 391.1 [M]⁺.

### Example 30: 2-[bis(4-chlorophenyl)methyl]-6-oxa-2,9-diazaspiro[4.5]decane (E30)

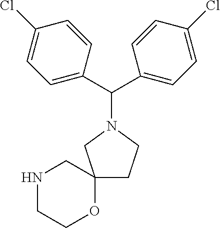 [SMILES: Clc1ccc(C(c2ccc(Cl)cc2)N2CCC3(CNCCO3)C2)cc1]

LiAlH₄ 2M in THF (0.14 mL, 0.288 mmol) was added to solution of 2-[bis(4-chlorophenyl)methyl]-6-oxa-2,9-diazaspiro[4.5]decan-8-one (p65, 75 mg, 0.192 mmol) in THF (5.5 mL) and the mixture was stirred at reflux for 1 h. The reaction was cooled down to −10° C., quenched with Na₂SO₄*10H₂O, diluted with EtOAc, filtered and concentrated. The crude material was purified by FC on NH column (eluent: Cy to Cy/EtOAc 50/50). 2-[bis(4-chlorophenyl)methyl]-6-oxa-2,9-diazaspiro[4.5]decane (E30, 40 mg, y=55%) was obtained as colourless oil.

MS (ES) (m/z): 377.14 [M+H]⁺.

1H NMR (CHLOROFORM-d): δ ppm 7.38 (d, 4H), 7.27 (dd, 4H), 4.19 (s, 1H), 3.65-3.73 (m, 1H), 3.55-3.65 (m, 1H), 2.77-2.85 (m, 4H), 2.54-2.65 (m, 3H), 2.43-2.52 (m, 1H), 1.83-2.01 (m, 2H)

### Preparation 66: 4-(aminomethyl)-1-benzylpiperidin-4-ol (P66)

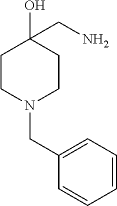 [SMILES: NCC1(O)CCN(Cc2ccccc2)CC1]

Step a:

To an ice-cooled mixture of NaH (60% dispersion in mineral oil, 0.55 g, 13.74 mmol) and trimethylsulfoxonium iodide (2.56 g, 11.62 mmol) DMSO (10 mL) was added keeping the mixture at 10° C. The mixture was stirred for 10 min at 10° C., then it was allowed to reach RT and left stirring at that temperature for 1 h. A solution of 1-benzylpiperidin-4-one (1.96 mL, 10.57 mmol) in DMSO (10 mL) was added via syringe. The mixture was stirred for 1.5 h at RT, diluted with Et₂O and quenched by the addition of saturated aqueous NH₄Cl. Phases were separated and aqueous one was backextracted with Et₂O. Combined organics were dried, filtered and concentrated under reduced pressure affording 6-benzyl-1-oxa-6-azaspiro[2.5]octane (2.05 g) as crude material that was used as such in the next step.

Step b:

To a stirred solution of 6-benzyl-1-oxa-6-azaspiro[2.5]octane (from step a, 2.05 g) in MeOH (12 mL), at 0° C., 28% aq. NH₄OH (26 mL), was added dropwise. Once the addition was complete, the ice-bath was removed and the resulting reaction mixture was stirred at RT overnight.

The reaction mixture was then concentrated under reduced pressure, the residue was taken up with DCM and 1N NaOH and aqueous phase was back extracted with DCM. Combined organics were dried and concentrated under reduced pressure. The crude material was purified by FC on NH column (eluent: DCM/MeOH from 100/0 to 90/10) affording 4-(aminomethyl)-1-benzylpiperidin-4-ol (p66, 1.48 g, y=67%).

MS (ES) (m/z): 221.2 [M+H]⁺.

### Preparation 67: N-[(1-benzyl-4-hydroxypiperidin-4-yl)methyl]-2-chloroacetamide (P67)

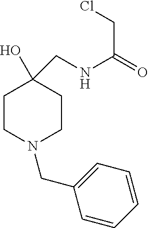 [SMILES: O=C(CCl)NCC1(O)CCN(Cc2ccccc2)CC1]

To a stirred solution of 4-(aminomethyl)-1-benzylpiperidin-4-ol (p66, 1.48 g, 6.7 mmol) in DCM (12 mL), at 0° C. and under a nitrogen atmosphere, TEA (1.87 mL, 13.4 mmol) was added followed by a solution of chloroacetylchloride (0.535 mL, 6.71 mmol) in 5 mL of DCM dropwise over 40 min.

Once the addition was complete, the reaction mixture was allowed to reach RT and left stirring at that temperature for 30 min, then diluted with DCM and saturated NH₄Cl. Phases were separated and the organic one was washed with brine, dried and concentrated under reduced pressure. The crude material was purified by FC on silica gel (eluent: DCM/MeOH from 100/0 to 95/5) affording N-[(1-benzyl-4-hydroxypiperidin-4-yl)methyl]-2-chloroacetamide (p67, 615 mg, y=31%).

MS (ES) (m/z): 297.1 [M+H]⁺.

### Preparation 68: tert-butyl 9-benzyl-1-oxa-4,9-diazaspiro[5.5]undecane-4-carboxylate (P68)

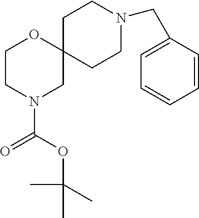 [SMILES: CC(C)(C)OC(=O)N1CCOC2(CCN(Cc3ccccc3)CC2)C1]

Step a:

To a stirred solution of N-[(1-benzyl-4-hydroxypiperidin-4-yl)methyl]-2-chloroacetamide (p67, 615 mg, 2.07 mmol) in THF (50 mL), at 0° C. and under a nitrogen atmosphere, NaH 60% dispersion in mineral oil (166 mg, 4.14 mmol) was added portionwise and then the ice-bath was removed. After 1 h at RT, the reaction mixture was concentrated under vacuum. The residue was diluted with AcOEt and water, and neutralized with 1N HCl. Phases were separated and the organic layer was dried, filtered and concentrated under reduced pressure affording 9-benzyl-1-oxa-4,9-diazaspiro[5.5]undecan-3-one (565 mg) that was used as such in next step.

Step b:

LiAlH₄ 1M in THF (3.25 mL, 3.25 mmol) was added to a solution of 9-benzyl-1-oxa-4,9-diazaspiro[5.5]undecan-3-one (from step a, 565 mg) in THF (25 mL) at 0° C. Once the addition was complete, the mixture was heated to reflux and stirred at that temperature for 40 min, then cooled down to −20° C. and quenched with Na₂SO₄*10H₂O. After quench, the mixture was left stirring at RT for 30 min, then filtered washing with AcOEt. Solvent was concentrated under reduced pressure affording 9-benzyl-1-oxa-4,9-diazaspiro[5.5]undecane (520 mg) that was used as such in next step.

Step c:

9-benzyl-1-oxa-4,9-diazaspiro[5.5]undecane (520 mg) was suspended in H₂O (120 mL) at RT then cooled at 0° C. Na₂CO₃ (218 mg, 2.15 mmol) was added followed by the dropwise addition of a solution of Boc₂O (461 mg, 2.11 mmol) in THF (10 mL). The mixture was stirred at 0° C. for 1 h, and then worked up extracting with EtOAc. The organic phase was dried, filtered and concentrated under reduced pressure. Crude material was purified by FC on silica gel (eluent: Cy to Cy/AcOEt 50:50) affording tert-butyl 9-benzyl-1-oxa-4,9-diazaspiro[5.5]undecane-4-carboxylate as clear oil (p68, 186 mg, y=26%)

MS (ES) (m/z): 347.3 [M+H]⁺.

### Preparation 69: tert-butyl 1-oxa-4,9-diazaspiro[5.5]undecane-4-carboxylate (P69)

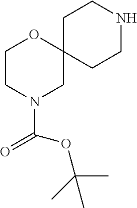 [SMILES: CC(C)(C)OC(=O)N1CCOC2(CCNCC2)C1]

To a solution of tert-butyl 9-benzyl-1-oxa-4,9-diazaspiro[5.5]undecane-4-carboxylate (p68, 186 mg, 0.537 mmol) in MeOH (4 mL) ammonium formate (133 mg, 2.11 mmol) and 10% Pd/C (57 mg) were added at RT then the mixture was stirred under reflux for 1 h. The mixture was cooled down to RT and filtered through a pad of celite washing with MeOH. Solvent was eliminated under reduced pressure affording tert-butyl 1-oxa-4,9-diazaspiro[5.5]undecane-4-carboxylate (p69, 159 mg, crude material) as white wax.

MS (ES) (m/z): 257.2 [M+H]⁺.

### Example 31: 9-[bis(4-fluorophenyl)methyl]-1-oxa-4,9-diazaspiro[5.5]undecane (E31)

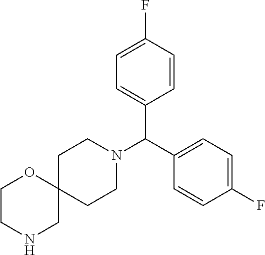 [SMILES: Fc1ccc(C(c2ccc(F)cc2)N2CCC3(CC2)CNCCO3)cc1]

Step a

To a solution of tert-butyl 9-benzyl-1-oxa-4,9-diazaspiro[5.5]undecane-4-carboxylate (p69, 30 mg, 0.117 mmol) in Acetonitrile (1 mL), 1-[chloro(4-fluorophenyl)methyl]-4-fluorobenzene (27 μL, 0.14 mmol) was added followed by K₂CO₃ (41 mg, 0.29 mmol). The mixture was heated to reflux for 7 hrs, then 0.2 eq of 1-[chloro(4-fluorophenyl)methyl]-4-fluorobenzene were further added, the mixture refluxed for further 1 h, and then left stirring at RT overnight. The day after, solid was filtered off washing with AcOEt and solvent was eliminated under reduced pressure affording tert-butyl 9-[bis(4-fluorophenyl)methyl]-1-oxa-4,9-diazaspiro[5.5]undecane-4-carboxylate (60 mg) used as such in next step.

Step b

TFA (0.25 mL) was added to a solution of tert-butyl 9-[bis(4-fluorophenyl)methyl]-1-oxa-4,9-diazaspiro[5.5]undecane-4-carboxylate (from step a, 60 mg) in 2 mL of DCM. The mixture was stirred for 1 h at RT, and then the solvent was removed under reduced pressure. The residue was loaded on SCX cartridge washing with MeOH and eluting with 1M NH₃ in MeOH affording 9-[bis(4-fluorophenyl)methyl]-1-oxa-4,9-diazaspiro[5.5]undecane (E31, 28 mg, y=66%) as oil.

MS (ES) (m/z): 359.18 [M+H]⁺.

¹H NMR (CHLOROFORM-d): δ ppm 7.36 (dd, 4H), 6.99 (t, 4H), 4.28 (s, 1H), 3.61-3.69 (m, 2H), 2.79-2.88 (m, 2H), 2.69-2.77 (m, 2H), 2.47 (d, 2H), 2.28 (t, 2H), 1.93 (d, 2H), 1.55 (m, 2H)

### Preparation 70: 4-bromo-1-[(tert-butoxy)carbonyl]piperidine-4-carboxylic acid (P70)

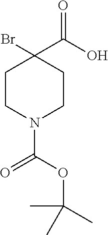 [SMILES: CC(C)(C)OC(=O)N1CCC(Br)(C(=O)O)CC1]

Bromoform (1.74 mL, 20 mmol) was added to a stirred solution of 1-Boc-4 piperidone (1 g, 5 mmol), benzyltriethylammonium chloride (0.125 g, 0.5 mmol) and LiOH H₂O (4.2 g, 100 mmol) in t-BuOH/H₂O (25/5 mL). The resulting mixture was vigorously stirred at RT for 72 hrs. The mixture was diluted with water (75 mL) and extracted with Et₂O (50 mL×2). The organic layer was discarded. The aqueous phase was cooled with an ice bath and the pH was adjusted to 1 with 20% HCl. The resulting precipitated was extracted with Et₂O (50 mL×2). The organic solution was dried and evaporated. The crude material was filtered through a silica cartridge eluting with EtOAc. After evaporation the resulting solid was triturated with hot n-Hexane. The white precipitate was filtered and dried to give 4-bromo-1-[(tert-butoxy)carbonyl]piperidine-4-carboxylic acid (p70, 650 mg, y=42%).

MS (ES) (m/z): 308.0 [M+H]⁺.

### Preparation 71: tert-butyl 5-oxo-1-thia-4,9-diazaspiro[5.5]undecane-9-carboxylate (P71)

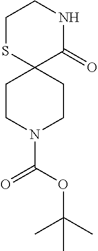 [SMILES: CC(C)(C)OC(=O)N1CCC2(CC1)SCCNC2=O]

Step a:

4-bromo-1-[(tert-butoxy)carbonyl]piperidine-4-carboxylic acid (p70, 650 mg, 2.1 mmol) was dissolved in a mixture of Toluene/MeOH (15/10 mL) and cooled with an ice bath. Trimethylsilyl-diazomethane (2.73 mL, 5.46 mmol) was added dropwise and then the reaction was stirred at RT for 3 hrs. The mixture was concentrated and Et₂O was added. The organic phase was washed with NH₄Cl and brine, dried and evaporated to afford 1-tert-butyl 4-methyl 4-bromopiperidine-1,4-dicarboxylate (670 mg) as colourless oil.

Step b:

2-amino-ethanthiol (235 mg, 2.07 mmol) was suspended in n-BuOH (15 mL) at 0° C. KOH (232 mg, 4.14 mmol) was added followed by 1-tert-butyl 4-methyl 4-bromopiperidine-1,4-dicarboxylate (from step a, 670 mg, 2.07 mmol). The cooling bath was removed and the reaction mixture was stirred at reflux for 48 hrs. The reaction mixture was cooled; the solids were removed by filtration. The filtrate was concentrated, re-dissolved with DCM and washed with 1N HCl and brine. Organic phase was then dried and concentrated under reduced pressure. The crude was purified by FC on silica gel (eluent: Cy to EtOAc) to afford tert-butyl 5-oxo-1-thia-4,9-diazaspiro[5.5]undecane-9-carboxylate (p71, 100 mg, y=17%) as white solid.

MS (ES) (m/z): 287.2 [M+H]⁺.

### Preparation 72: 9-[bis(4-fluorophenyl)methyl]-1-thia-4,9-diazaspiro[5.5]undecan-5-one (P72)

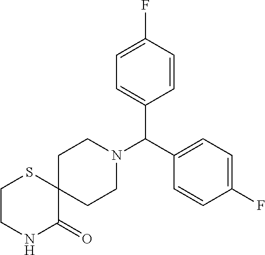 [SMILES: O=C1NCCSC12CCN(C(c1ccc(F)cc1)c1ccc(F)cc1)CC2]

Step a

tert-butyl 5-oxo-1-thia-4,9-diazaspiro[5.5]undecane-9-carboxylate (p71, 100 mg, 0.34 mmol) was dissolved in DCM (3 mL) and 1N HCl in Et₂O was added dropwise. The solution was stirred at RT overnight, and then the solvent was evaporated to afford 1-thia-4,9-diazaspiro[5.5]undecan-5-one as hydrochloric salt (80 mg).

Step b

Chlorobis(4-fluorophenyl)methane (0.08 mL, 0.43 mmol) was added to a stirred mixture of 1-thia-4,9-diazaspiro[5.5]undecan-5-one hydrochloric salt (from step a, 80 mg) and K₂CO₃ (173 mg, 1.25 mmol) in Acetonitrile (5 mL). The mixture was stirred overnight at reflux. The reaction mixture was cooled to RT, filtered using EtOAc and solvent evaporated. The crude material was charged on SCX cartridge washing with MeOH and eluting with 1M NH₃ in MeOH then purified by FC on silica gel (eluent: DCM to DCM/MeOH 95/5) affording 9-[bis(4-fluorophenyl)methyl]-1-thia-4,9-diazaspiro[5.5]undecan-5-one (p72, 100 mg, y=76%) as white foam.

MS (ES) (m/z): 389.2 [M+H]⁺.

### Example 32: 9-[bis(4-fluorophenyl)methyl]-1-thia-4,9-diazaspiro[5.5]undecane (E32)

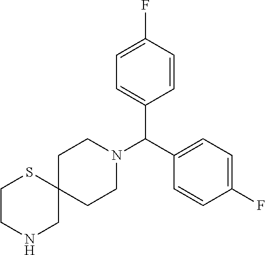 [SMILES: Fc1ccc(C(c2ccc(F)cc2)N2CCC3(CC2)CNCCS3)cc1]

9-[bis(4-fluorophenyl)methyl]-1-thia-4,9-diazaspiro[5.5]undecan-5-one (p72, 100 mg, 0.257 mmol) was dissolved in THF under Argon, LiAlH₄ 1M in THF (0.257 mL, 0.257 mmol) was added dropwise and the solution was then heated at 60° C. for 30 min. The reaction was cooled with an ice bath, Na₂SO₄*10H₂O was added portion wise and the mixture stirred for 30 min. The mixture was filtered washing with EtOAc. After solvent evaporation 9-[bis(4-fluorophenyl)methyl]-1-thia-4,9-diazaspiro[5.5]undecane (E32, 85 mg, y=75%) was obtained as white foam.

MS (ES) (m/z): 375.2 [M+H]⁺.

¹H NMR (CHLOROFORM-d): δ ppm 7.32-7.41 (m, 4H), 6.99 (t, 4H), 4.31 (s, 1H), 3.05-3.15 (m, 2H), 2.97 (s, 2H), 2.48-2.65 (m, 4H), 2.36 (d, 2H), 1.89 (d, 2H), 1.81-1.73 (m, 2H)

Biological Methods

The ability of the compounds of formula I to inhibit dopamine transporters may be determined using the following biological assays:

Measure of Affinity to the Human Transporters DAT, NET and SERT

The affinities of the compounds of the invention for the human dopamine transporter (DAT), human norepinephrine transporter (NET) and for the human serotonin transporter (SERT) may be determined by the assays described below. Affinity is expressed in terms of inhibition constant (Ki) of the compounds of the invention for DAT, NET and SERT, and it is typically calculated from the IC₅₀ values obtained in competition experiments using Cheng and Prusoff equation (Cheng and Prusoff, Biochem. Pharmacol. 22:3099, 1973). In the context of the present invention pKi values (corresponding to the antilogarithm of Ki) are used instead of Ki; pKi are only estimated to be accurate to about 0.3 log unit.

Scintillation Proximity Assay (SPA) for Human DAT, NET and SERT Binding

a) Membrane Preparation

Chinese Hamster Ovary (CHO) cells stably expressing either human DAT (hDAT-CHO) or human NET (hNET-CHO) or human SERT (hSERT-CHO) are used for the membrane preparations for radioligand binding assays using Scintillation proximity Assay (SPA) technique. Each cell line is cultured independently in F-12K Nutrient Mixture containing 10% of Fetal Bovine Serum (FBS) supplemented with 450 μg/ml G-418. When cells are at 70-80% of confluence 3 mM Na Butyrate was added to the cell culture medium. After 24 h of incubation, the culture medium was removed and the cells detached with Versene (DAT) or by scraping (NET and SERT). Cell suspension is centrifuged at 41,000 g for 10 minutes at 4° C. The resultant pellets are re-suspended in 15 volumes of Ice-cold buffer (20 mM HEPES, 145 mM NaCl, 5 mM KCl, pH 7.3), homogenized using an Ultra Turrax homogeniser and centrifuged as before. The resultant membrane pellets are re-suspended in up to 15 volume of ice-cold buffer, incubated for 20 minutes at 37° C. and centrifuged as before at 41,000 g. The final membrane pellets are re-suspended into 5-10 volumes of ice-cold buffer, dispensed into 0.5 ml aliquots and stored at −80° C. until use. Protein concentration for each preparation is determined using Bio-Rad Protein Assay kit.

b) Competition Binding Experiments Using Scintillation Proximity Assay (SPA) for Human DAT, NET and SERT

The affinity of the compounds of the invention to the human DAT or NET or SERT transporters is assessed by using the [³H]WIN-35,428 or [³H]nisoxetine or [³H]citalopram binding assays in recombinant human DAT, NET and SERT membranes with the SPA technology. The final assay volume is 50 μL in 384 well plates.

Briefly, 0.5 μL of test compound in neat DMSO or 0.5 μL of DMSO for total binding (TB) or 0.5 μL of indatraline 1 mM (10 μM final concentration) for non specific binding (NSB) are added to the assay plate. 50 μL of the SPA mixture is added to each well, containing: 30 μg/mL or 10 μg/mL or 25 μg/mL DAT, NET, SERT membranes, respectively; 5 nM [³H]WIN-35,428 or 5 nM [³H]nisoxetine or 1 nM [³H]citalopram, for DAT, NET, SERT assay, respectively; 2.5 mg/mL or 1 mg/mL or 4 mg/mL WGA-PVT SPA beads (PerkinElmer RPNQ0001, for DAT, NET, SERT assay, respectively. All components are added to Assay Buffer (20 mM HEPES pH 7.4, 145 mM NaCl, 5 mM KCl, 0.01% Pluronic F-127). 0.02% BSA was used for DAT binding only. Plates are sealed with Topseal A and centrifuged 1 min, 800 rpm. Plates are loaded into a 1450 Microbeta TriLux (Perkin-Elmer) plate reader and the radioactivity counted after at least 4 hrs or overnight incubation at room temperature. Curve fitting and IC₅₀ estimations are performed using a four parameter model in XLfit (IDBS, Guilford, UK) for Microdoft Excel (Microsoft, Redmond, Wash.).

Uptake Functional Assay on hDAT-CHO Cells

The potency of the compounds of the invention in blocking the DAT function is measured using an uptake assay in a recombinant CHO cell line expressing human DAT (hDAT-CHO). Potency is measured in terms of plC₅₀ by testing the compounds of invention for the inhibition of [³H]-dopamine uptake in DAT-CHO cells using a SPA technology in 384 well format.

Briefly, on the days of the experiment hDAT-CHO cells are detached using Versene and added (75,000 cells/mL) to the SPA Mixture, which contains the following components in Assay Buffer (20 mM HEPES, 145 mM NaCl, 5 mM KCl, 2 mM CaCl₂, 1 mM MgCl₂ and 1 g/L glucose, pH 7.3): 0.02% w/v of Pluronic F127, 2 mg/mL SPA Imaging beads (RPNQ0260, PerkinElmer), 10 μM pargyline and 80 nM of [³H]-dopamine. The SPA Mixture is added 50 μl/well to 384 well plates containing 0.5 μL/well of test compound in neat DMSO or 0.5 μL of DMSO (control uptake) or 0.5 μL of the standard inhibitor indatraline (at 10 μM final in the assay). Plates are sealed with a Top-seal A and read using Viewlux instrument (Perkin-Elmer) at 15-30 min time intervals. The first highest signal is used for data analysis.

Measure of the Effect on hERG Channel by Tail Current Recording Using In Vitro Rapid ICE™

The potency of the compounds of the invention in inhibiting human ERG potassium channel (hERG) tail current is assessed in a recombinant HEK293 cell line stably transfected with hERG cDNA using Rapid ICE™ (Rapid Ion Channel Electrophysiology) assay. Rapid ICE™ is an automated patch-clamp assay utilizing the PatchXpress 7000A system (Molecular Devices Corporation) or the QPatch HTX system (Sophion Bioscience A/S).

Briefly cells are cultivated for 24 to 72 hours before recordings in minimum essential medium supplemented with 10% FBS, 1% non-essential amino acids, 1% sodium pyruvate, 2 mM L-glutamine. The day of the experiment cells are detached with TrypLE and prepared to be loaded on the instrument. For PatchXpress cells are finally resuspended in 150 μl of Extracellular Buffer whereas for QPatch cells are resuspended in 7 ml Serum-Free Media containing 25 mM Hepes and Soybean trypsin inhibitor and immediately placed in the cell storage tank of the machine. The composition of the Extracellular Buffer is (mM): NaCl 137; KCl 4; CaCl2 1.8; MgCl2 1.0; D-glucose 10; N 2 hydroxyethylpiperazine-N′-2-ethanesulfonic acid (HEPES) 10; pH 7.4 with 1 M NaOH. The composition of the pipette solution is (mM): KCl 130; MgCl2 1.0; Ethylene glycol-bis(R-aminoethyl ether)-N,N,N′,N′-tetraacetic acid (EGTA) 5; MgATP 5; HEPES 10; pH 7.2 with 1 M KOH. The voltage protocol includes the following steps: step from −80 mV to −50 mV for 200 ms, +20 mV for 4.8 s, step to −50 mV for 5 s then step to the holding potential of −80 mV. Compounds of the invention are dissolved in DMSO and diluted in Extracellular Buffer to achieve final test concentrations (0.1, 1 and 10 μM) in 0.1% DMSO. The voltage protocol is run and recorded continuously during the experiment. The vehicle, corresponding to 0.1% DMSO in Extracellular Buffer, is then applied for 3 min followed by the test substance in triplicate. The standard combined exposure time is 5 min. The average of tail current amplitude values recorded from 4 sequential voltage pulses is used to calculate for each cell the effect of the test substance by calculating the residual current (% control) compared with vehicle pre-treatment. Data are reported as % inhibition for each concentration tested and IC₅₀ values are estimated using DataXpress or QPatch software. At least two cells are tested, more if results diverge.

Representative compounds of the present invention were tested according to the procedure described above, with results as listed in Table 1 below.

TABLE 1

pIC₅₀

| Example | DAT | NET | SERT | hERG |
|---|---|---|---|---|
| 1 | 6.89 | 5.42 | 4.65 | 5.7 |
| 2 | 6.43 | 4.65 | 4.66 | 5.1 |
| 3 | 7.15 | 5.35 | 4.89 | 5.6 |
| 4 | 6.71 | 4.74 | 4.73 | 5.8 |
| 5 | 6.84 | 5.49 | 5.13 | — |
| 6 | 6.51 | 5.35 | 5.47 | — |
| 7 | 6.79 | 5.67 | 4.85 | — |
| 8 | 6.88 | 5.86 | 5.13 | — |
| 9 | 6.58 | 5.62 | 4.97 | 5.3 |
| 10 | 6.83 | 5.19 | 4.81 | 5.3 |
| 11 | 6.65 | 5.45 | 4.89 | — |
| 12 | 6.52 | 5.10 | 4.77 | — |
| 13 | 6.76 | 5.37 | 4.71 | — |
| 14 | 6.70 | 5.76 | 4.65 | — |
| 15 | 6.69 | 5.70 | 5.32 | — |
| 16 | 7.31 | 5.20 | 5.18 | 5.6 |
| 17 | 7.64 | 5.97 | 4.88 | 5.5 |
| 18 | 7.52 | 5.73 | 5.03 | 5.5 |
| 19 | 6.69 | 5.48 | 5.75 | — |
| 20 | 7.54 | 5.70 | 5.68 | — |
| 21 | 6.73 | 5.22 | 5.17 | — |
| 22 | 6.96 | 5.24 | 5.19 | — |
| 23 | 6.94 | 5.30 | 5.37 | — |
| 24 | 6.27 | 6.53 | 4.96 | — |
| 25 | 6.44 | 5.62 | 5.80 | — |
| 26 | 6.87 | 5.70 | 5.28 | — |
| 27 | 6.83 | 5.77 | 5.46 | 5.6 |
| 28 | 6.50 | 5.91 | 6.17 | — |
| 29 | 6.97 | 5.86 | 5.16 | 5.2 |
| 30 | 6.82 | 5.62 | 6.04 | — |
| 31 | 7.33 | 5.69 | 5.58 | 5.5 |
| 32 | 7.23 | 5.61 | 5.63 | — |

While the foregoing specification teaches the principles of the present invention, with examples provided for the purpose of illustration, it will be understood that the practice of the invention encompasses all of the usual variations, adaptations and/or modifications as come within the scope of the following claims.

### REFERENCES

1 Wise R A, Annu Rev Neurosci. 1996; 19: 319-340
2 Cohen N J et al., Psychopharmacologia. 1971; 22(3): 282-294
3 Leibowitz S F et al., Brain Res Bull. 1986; 17(5): 681-689.
4 Hartmann E et al., Psychopharmacology (Berl). 1976 10; 50(2): 171-175
5 Lader M H, J Clin Psychiatry. 1996; 57 Suppl 2: 39-44
6 Montejo-González A L et al., J Sex Marital Ther. 1997; 23(3): 176-194
7 Olfson M et al., Arch Gen Psychiatry. 2006 August; 63(8): 865-872
8 Dworkin N, J Am Acad Child Adolesc Psychiatry. 2005; 44(6): 510
9 Denolle T et al., Clin Pharmacol Ther. 1999; 66(3): 282-287
10 Nieoullon A, Prog Neurobiol. 2002; 67(1): 53-83
11 Cornish R S et al., Pharm Res. 2005; 22(4): 603-612
12 Cook E H Jr et al., Am J Hum Genet. 1995; 56(4): 993-998
13 Van Gaalen M M et al., Biol Psychiatry. 2006; 60(1): 66-73
14 Yoon et al., J Neurol Sci. 2007; 255(1-2): 50-56
15 Cheon et al., Psychiatry Res. 2004; 130(1): 85-95
16 Kim C H et al., Eur J Nucl Med Mol Imaging. 2003; 30(12): 1637-1643
17 Grigorenko E L et al., Aggress Behav. 2010; 36(3): 158-176
18 Amsterdam et al., J Affect Disord. 2012; 141(2-3): 425-431
19 Hsiao et al., Psychiatry Res. 2013; 211(1): 72-77
20 Baldwin D S et al., Br J Psychiatry. 2013; 202: 396-397
21 Abler B et al., Neuropsychopharmacology. 2011; 36(9): 1837-1847
22 Segman et al., Mol Psychiatry. 2002; 7(8): 903-7
23 Devos D et al., J Neurol Neurosurg Psychiatry. 2007; 78(5): 470-475
24 Espay et al., Neurology. 2011; 76(14): 1256-1262
25 Auriel et al., Clin Neuropharmacol. 2006; 29(1): 15-17
26 Baumann M H et al., J Pharmacol Exp Ther. 1994; 271(3): 1216-1222
27 Rothman R B et al., Pharmacol Biochem Behav. 1991; 40(2): 387-397
28 Wang G J et al., Obesity (Silver Spring) 2011; 19(8): 1601-1608
29 Michaelides M et al., Int Rev Psychiatry. 2012; 24(3): 211-218
30 Bello et al., Brain Res Bull. 2006; 70(4-6): 422-429
31 Shinohara M et al., J Psychiatry Neurosci. 2004; 29(2): 134-137
32 Slama et al., Diabete Metab. 1978; 4(3): 193-199
33 Remy P et al., Curr Opin Neurol. 2003; 16 Suppl 2: S37-41
34 Berrios G E, Compr Psychiatry 1990; 31(2): 140-151
35 Harris J D, Curr Opin Support Palliat Care 2008; 2(3): 180-186
36 Lacerda et al., J Cardiovasc Electrophysiol. 2010; 21(3): 301-310
37 Campbell V C et al., J Pharmacol Exp Ther. 2005; 315(2): 631-640
38 Zou M F et al., J Med Chem. 2006; 49(21): 6391-6399
39 Li S M et al., J Pharmacol Exp Ther. 2011; 336(2): 575-585

1. A compound according to formula I 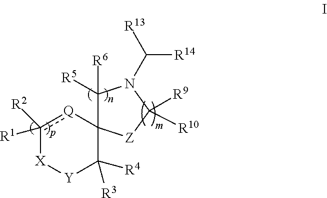
wherein:
Q is selected from CR⁷R⁸, C═O, C═N—OH, C═N—O-alkyl, NH, N-cycloalkyl, N-alkyl, S(O)_{q} and O;
X is selected from C═O, CR¹¹R¹², NH, N-cycloalkyl and N-alkyl;
Y is selected from CR¹¹R¹², NH, N-alkyl, N-cycloalkyl, S(O)_{q} and O;
wherein:
X is C═O or CR¹¹R¹² when Y is O, S(O)_{q}, NH, N-alkyl or N-cycloalkyl;
X is C═O or CR¹¹R¹² when p is 0 and Q is S(O)_{q}, O, NH, N-cycloalkyl or N-alkyl;
Y is CR¹¹R¹² when X is NH, N-cycloalkyl or N-alkyl;
Q is selected from CR⁷R⁸, C═O, C═N—OH and C═N—O-alkyl when n is 0;
Q is CR⁷R⁸ when p is 0 and X is NH, N-cycloalkyl or N-alkyl;
Q is CR⁷R⁸, O, NH, N-cycloalkyl or N-alkyl when p is 0 and X is C═O;
Q is NH, N-cycloalkyl or N-alkyl and X is CR¹¹R¹² when Y is O or S(O)_{q}, and
at least one of Q, X and Y is NH, N-cycloalkyl or N-alkyl;
Z is selected from CR¹¹R¹², O and S; wherein Z is CR¹¹R¹² when Q is O, S(O)_{q}, NH, N-cycloalkyl or N-alkyl, or when m is 0, or when n is 0;
R¹ is selected from H, OH, alkyl, F, Cl, and alkoxy;
R² is selected from H, OH, alkyl, F, Cl, and alkoxy;
or R¹ and R² may together form ═O;
R³ and R⁴ are independently selected from H, OH, alkoxy and alkyl;
or R³ and R⁴ may both be O, wherein said O atoms are linked by an alkylene group to form a straight chain or branched alkylenedioxy group;
or R³ and R⁴ may together form ═O;
R⁵ and R⁶ are independently selected from H and alkyl;
or R⁵ and R⁶ may together form ═O;
R⁷ is selected from H, F, Cl, OH and alkoxy;
R⁸ is absent or is selected from H, F, Cl, OH and alkoxy;
or R⁷ and R⁸ may both be O, wherein said O atoms are linked by an alkylene group to form an alkylenedioxy group;
R¹³ is substituted phenyl;
R¹⁴ is substituted phenyl or unsubstituted phenyl;
R⁹, R¹⁰, R¹¹, R¹², R¹⁵ and R¹⁶ are independently selected from H and alkyl;
q is 0, 1 or 2;
n is 0, 1 or 2, wherein n is 0 or 1 when m is 2, and n is 1 or 2 when m is 0
m is 0, 1 or 2, wherein m is 0 or 1 when n is 2, and m is 1 or 2 when n is 0;
p is 0, 1 or 2; wherein p is 1 or 2 when n is 2;
- - - - is absent or represents a bond; wherein when - - - - is a bond R² is absent, Q is CR⁷R⁸, R⁸ is absent, and p is 1 or 2;
alkyl is a linear saturated hydrocarbon having up to 6 carbon atoms (C₁-C₆) or a branched saturated hydrocarbon of between 3 and 6 carbon atoms (C₃-C₆); alkyl may optionally be substituted with 1, 2, 3, 4 or 5 substituents independently selected from cycloalkyl, S-alkyl, S(O)alkyl, S(O)₂alkyl, cycloalkyl, heterocyclyl, alkoxy, OH, —CN, CF₃, COOR¹⁵, CONR¹⁵R¹⁶, F, Cl, NR¹⁵COR¹⁶ and NR¹⁵R¹⁶;
alkylene is a bivalent C₁₋₃ straight-chained alkyl radical or a bivalent C₃₋₄ branched alkyl radical, wherein alkylene may optionally be substituted with 1 or 2 substituents selected from S-alkyl, S(O)alkyl, S(O)₂alkyl, heterocyclyl, alkoxy, OH, —CN, CF₃, COOR¹⁵, CONR¹⁵R¹⁶, F, Cl, NR¹⁵COR¹⁶ and NR¹⁵R¹⁶;
alkoxy is a linear O-linked hydrocarbon of between 1 and 6 carbon atoms (C₁-C₆) or a branched O-linked hydrocarbon of between 3 and 6 carbon atoms (C₃-C₆); alkoxy may optionally be substituted with 1, 2, 3, 4 or 5 substituents independently selected from S-alkyl, S(O)alkyl, S(O)₂alkyl, alkyl, OH, —CN, CF₃, COOR¹⁵, CONR¹⁵R¹⁶, F, Cl, NR¹⁵COR¹⁶ and NR¹⁵R¹⁶;
cycloalkyl is a monocyclic saturated hydrocarbon of between 3 and 7 carbon atoms; cycloalkyl may optionally be substituted with 1, 2, 3, 4 or 5 substituents independently selected from S-alkyl, S(O)alkyl, S(O)₂alkyl, alkyl, alkoxy, OH, —CN, CF₃, COOR¹⁵, CONR¹⁵R¹⁶, F, Cl, NR¹⁵COR¹⁶ and NR¹⁵R¹⁶;
substituted phenyl is a phenyl group substituted with 1, 2 or 3 substituents independently selected from alkyl, cycloalkyl, heterocyclyl, alkoxy, S-alkyl, S(O)alkyl, S(O)₂alkyl, OH, F, Cl, —CN, OCF₃, CF₃, NR¹³COR¹⁴ and NR¹⁵R¹⁶;
heterocyclyl is a monocyclic ring which is saturated or partially unsaturated, containing, where possible, 1 or 2 ring members independently selected from N, S, O and NR¹⁵ and 2 to 5 carbon atoms; heterocyclyl may optionally be substituted with 1, 2 or 3 substituents independently selected from alkyl, cycloalkyl, alkoxy, S-alkyl, S(O)alkyl, S(O)₂alkyl, oxo, OH, F, Cl, —CN, OCF₃, CF₃, NR¹⁵COR¹⁶ and NR¹⁵R¹⁶;
and tautomers, stereoisomers (including enantiomers, diastereoisomers and racemic and scalemic mixtures thereof), pharmaceutically acceptable salts and solvates thereof;
wherein:
R¹ is not OH or alkoxy when Q is NH, N-alkyl, N-cycloalkyl or when X is NH, N-alkyl or N-cycloalkyl; and
R² is not OH or alkoxy when Q is NH, N-alkyl, N-cycloalkyl or when X is NH, N-alkyl or N-cycloalkyl; and
R³ is not OH or alkoxy when Y is O, NH, N-alkyl or N-cycloalkyl; and
R⁴ is not OH or alkoxy when Y is O, NH, N-alkyl or N-cycloalkyl.

2. The compound of claim 1 wherein R¹³ and R¹⁴ are para-fluoro-phenyl.

3. The compound of claim 1 wherein p is 1.

4. The compound of claim 1 wherein m is 1 or 2 and n is 1 or 2, wherein n is 1 when m is 2; and m is 1 when n is 2.

5. The compound of claim 1 wherein n is 1 and m is 1.

6. The compound of claim 1 wherein Z is CH₂.

7. The compound of claim 1 wherein X is CH₂ and Y is NH.

8. The compound of claim 1 wherein Q is selected from CR⁷R⁸, S and O.

9. The compound of claim 1 wherein Q is CR⁷R⁸.

10. The compound of claim 1 selected from:
2-[bis(4-fluorophenyl)methyl]-2,7-diazaspiro[4.5]decan-10-one;
2-[bis(4-fluorophenyl)methyl]-7-methyl-2,7-diazaspiro[4.5]decan-10-one;
2-[bis(4-fluorophenyl)methyl]-2,7-diazaspiro[4.5]decan-10-ol;
2-[bis(4-fluorophenyl)methyl]-7-methyl-2,7-diazaspiro[4.5]decan-10-ol;
2-[bis(4-fluorophenyl)methyl]-10-methoxy-2,7-diazaspiro[4.5]decane;
8-[bis(4-fluorophenyl)methyl]-1,4-dioxa-8,12-diazadispiro[4.0.4⁶.4⁵]tetradecane;
(5R)-2-[bis(4-fluorophenyl)methyl]-2,7-diazaspiro[4.5]decan-10-one;
(5S)-2-[bis(4-fluorophenyl)methyl]-2,7-diazaspiro[4.5]decan-10-one;
2-[bis(4-fluorophenyl)methyl]-2,7-diazaspiro[4.5]decan-10-ol;
2-[bis(4-fluorophenyl)methyl]-N-methoxy-2,7-diazaspiro[4.5]decan-10-imine;
(5R,10E)-2-[bis(4-fluorophenyl)methyl]-N-methoxy-2, 7-diazaspiro[4.5]decan-10-imine;
(5S,10E)-2-[bis(4-fluorophenyl)methyl]-N-methoxy-2,7-diazaspiro[4.5]decan-10-imine;
N-[2-[bis(4-fluorophenyl)methyl]-2,7-diazaspiro[4.5]decan-10-ylidene]hydroxylamine
N-[(5R,10E)-2-[bis(4-fluorophenyl)methyl]-2,7-diazaspiro[4.5]decan-10-ylidene]hydroxylamine;
N-[(5S,10E)-2-[bis(4-fluorophenyl)methyl]-2,7-diazaspiro[4.5]decan-10-ylidene]hydroxylamine;
2-[bis(4-fluorophenyl)methyl]-10,10-difluoro-2,7-diazaspiro[4.5]decane;
(5R)-2-[bis(4-fluorophenyl)methyl]-10,10-difluoro-2,7-diazaspiro[4.5]decane;
(5S)-2-[bis(4-fluorophenyl)methyl]-10,10-difluoro-2,7-diazaspiro[4.5]decane;
2-[bis(4-fluorophenyl)methyl]-10-fluoro-2,7-diazaspiro[4.5]dec-9-ene;
(5R)-2-[bis(4-fluorophenyl)methyl]-10-fluoro-2,7-diazaspiro[4.5]dec-9-ene;
(5S)-2-[bis(4-fluorophenyl)methyl]-10-fluoro-2,7-diazaspiro[4.5]dec-9-ene;
2-[bis(4-chlorophenyl)methyl]-2,7-diazaspiro[4.5]decan-10-one;
8-[bis(4-fluorophenyl)methyl]-1,4-dioxa-8,13-diazadispiro[4.0.4⁶.4⁵]tetradecane;
2-[bis(4-fluorophenyl)methyl]-2,8-diazaspiro[4.5]decan-6-ol;
2-[bis(4-fluorophenyl)methyl]-2,7-diazaspiro[4.5]decane;
2-[bis(4-fluorophenyl)methyl]-7-methyl-2,7-diazaspiro[4.5]decane;
7-[bis(4-fluorophenyl)methyl]-2,7-diazaspiro[4.5]decane;
2-[bis(4-fluorophenyl)methyl]-2,9-diazaspiro[5.5]undecane;
9-[bis(4-fluorophenyl)methyl]-2,9-diazaspiro[5.5]undecane;
2-[bis(4-fluorophenyl)methyl]-2,8-diazaspiro[4.5]decane;
2-[bis(4-chlorophenyl)methyl]-2, 8-diazaspiro[4.5]decane;
2-[bis(4-fluorophenyl)methyl]-6-oxa-2,9-diazaspiro[4.5]decane;
2-[bis(4-chlorophenyl)methyl]-6-oxa-2,9-diazaspiro[4.5]decane;
9-[bis(4-fluorophenyl)methyl]-1-oxa-4,9-diazaspiro[5.5]undecane;
9-[bis(4-fluorophenyl)methyl]-1-thia-4,9-diazaspiro[5.5]undecane;
and pharmaceutically acceptable salts and solvates thereof.

11. The compound of claim 1 selected from:
2-[bis(4-fluorophenyl)methyl]-2,7-diazaspiro[4.5]decan-10-ol;
2-[bis(4-fluorophenyl)methyl]-10,10-difluoro-2,7-diazaspiro[4.5]decane;
(5R)-2-[bis(4-fluorophenyl)methyl]-10,10-difluoro-2,7-diazaspiro[4.5]decane;
(5S)-2-[bis(4-fluorophenyl)methyl]-10,10-difluoro-2,7-diazaspiro[4.5]decane;
2-[bis(4-fluorophenyl)methyl]-10-fluoro-2,7-diazaspiro[4.5]dec-9-ene;
(5R)-2-[bis(4-fluorophenyl)methyl]-10-fluoro-2,7-diazaspiro[4.5]dec-9-ene;
(5S)-2-[bis(4-fluorophenyl)methyl]-10-fluoro-2,7-diazaspiro[4.5]dec-9-ene;
8-[bis(4-fluorophenyl)methyl]-1,4-dioxa-8,13-diazadispiro[4.0.4⁶.4⁵]tetradecane;
2-[bis(4-fluorophenyl)methyl]-2,7-diazaspiro[4.5]decane;
and pharmaceutically acceptable salts and solvates thereof.

12. A pharmaceutical composition comprising the compound of claim 1 and a pharmaceutically acceptable excipient.

13. (canceled)

14. A method of treating or preventing a condition, disease or disorder ameliorated by inhibition of a dopamine transporter, the method comprising administering the compound of claim 1 to a subject in need thereof.

15. The method of claim 14, wherein said condition, disease or disorder is selected from sexual dysfunction, affective disorders, anxiety, depression, Tourette syndrome, Angelman syndrome, attention deficit disorder (ADD), attention deficit hyperactivity disorder (ADHD), obesity, pain, obsessive-compulsive disorder, movement disorders, CNS disorders, sleep disorders, narcolepsy, conduct disorder, substance abuse (including smoking cessation), eating disorders, chronic fatigue and impulse control disorders.

16. The method of claim 14, wherein said condition, disease or disorder is selected from attention deficit disorder (ADD), attention deficit hyperactivity disorder (ADHD) or binge-eating.
